SEQUENCE LISTING (16 sequences)

SEQ ID NO: 1 - protein, 219 aa, synthetic construct
  REGION 1..219: note = Antibody light chain
DIQMTQSPSSLSASVGDRVTITCRSSQNIVHSNGNTYLDWYQQTPGKAPKLLIYKVSNRFSGVPSRFSGSGSGTDFTFTISSLQPEDIATYYCFQYSHVPWTFGQGTKLQITREVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 2 - protein, 222 aa, synthetic construct
  REGION 1..222: note = Antibody heavy chain
QVQLQQSGAEVKKPGSSVKVSCKASGYTFTNYYIYWVRQAPGQGLEWIGGINPTSGGSNFNEKFKTRVTITADESSTTAYMELSSLRSEDTAFYFCTRQGLWFDSDGRGFDFWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVP

SEQ ID NO: 3 - protein, 213 aa, synthetic construct
  REGION 1..213: note = Antibody light chain
DILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGA

SEQ ID NO: 4 - protein, 221 aa, synthetic construct
  REGION 1..221: note = Antibody heavy chain
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKS

SEQ ID NO: 5 - protein, 16 aa, synthetic construct
  REGION 1..16: note = CDR sequence
RSSQNIVHSNGNTYLD

SEQ ID NO: 6 - protein, 7 aa, synthetic construct
  REGION 1..7: note = CDR sequence
KVSNRFS

SEQ ID NO: 7 - protein, 9 aa, synthetic construct
  REGION 1..9: note = CDR sequence
FQYSHVPWT

SEQ ID NO: 8 - protein, 5 aa, synthetic construct
  REGION 1..5: note = CDR sequence
NYYIY

SEQ ID NO: 9 - protein, 17 aa, synthetic construct
  REGION 1..17: note = CDR sequence
GINPTSGGSNFNEKFKT

SEQ ID NO: 10 - protein, 14 aa, synthetic construct
  REGION 1..14: note = CDR sequence
QGLWFDSDGRGFDF

SEQ ID NO: 11 - protein, 11 aa, synthetic construct
  REGION 1..11: note = CDR sequence
RASQSIGTNIH

SEQ ID NO: 12 - protein, 5 aa, synthetic construct
  REGION 1..5: note = CDR sequence
ASEIS

SEQ ID NO: 13 - protein, 9 aa, synthetic construct
  REGION 1..9: note = CDR sequence
QQNNNWPTT

SEQ ID NO: 14 - protein, 5 aa, synthetic construct
  REGION 1..5: note = CDR sequence
NYGVH

SEQ ID NO: 15 - protein, 16 aa, synthetic construct
  REGION 1..16: note = CDR sequence
VIWSGGNTDYNTPFTS

SEQ ID NO: 16 - protein, 11 aa, synthetic construct
  REGION 1..11: note = CDR sequence
ALTYYDYEFAY

(19) United States
(12) Patent Application Publication
(10) Pub. No.: US 2023/0312675 A1
(43) Pub. Date: Oct. 5, 2023

(54) CHIMERIC ANTIGEN RECEPTORS (CAR) AND METHODS FOR MAKING AND USING THE SAME

(71) Applicant: BOARD OF REGENTS, THE UNIVERSITY OF TEXAS SYSTEM, Austin, TX (US)

(72) Inventors: Laurence J.N. COOPER, Houston, TX (US); Hillary Gibbons CARUSO, Houston, TX (US); Simon OLIVARES, Houston, TX (US); Sonny ANG, Houston, TX (US)

(73) Assignee: BOARD OF REGENTS, THE UNIVERSITY OF TEXAS SYSTEM, Austin, TX (US)

(21) Appl. No.: 18/153,025

(22) Filed: Jan. 11, 2023

Related U.S. Application Data
(63) Continuation of application No. 16/600,806, filed on Oct. 14, 2019, now abandoned.
Application No. 16/600,806 is a division of application No. 15/305,996, filed on Oct. 21, 2016, now abandoned.
(60) Provisional application No. 61/983,298, filed on Apr. 23, 2014.
(60) Provisional application No. 61/983,103, filed on Apr. 23, 2014.

(51) Int. Cl. C07K 14/725; C07K 14/705; C07K 16/28; A61K 39/00; C12N 5/0783; A61K 39/395; C07K 14/715

(52) U.S. Cl. CPC C07K 14/7051; A61K 39/0011; A61K 39/001104; A61K 39/001106; A61K 39/001107; A61K 39/001109; A61K 39/00111; A61K 39/001112; A61K 39/001113; A61K 39/001119; A61K 39/001124; A61K 39/001126; A61K 39/001129; A61K 39/001151; A61K 39/001164; A61K 39/001168; A61K 39/00117; A61K 39/001171; A61K 39/001174; A61K 39/001181; A61K 39/001182; A61K 39/00119; A61K 39/001194; A61K 39/39558; C07K 14/70521; C07K 14/7153; C07K 16/2809; C07K 16/2863; C12N 5/0638; A61K 2039/505; A61K 2039/5156; A61K 2039/5158; C07K 2317/622; C07K 2317/64; C07K 2317/73; C07K 2317/92; C07K 2319/02; C07K 2319/03; C07K 2319/30; C07K 2319/33; C07K 2319/70; C12N 2501/2302; C12N 2501/515

(57) ABSTRACT

Chimeric antigen receptors (CARs) and CAR-expressing T cells are provided that can specifically target cells that express an elevated level of a target antigen. Likewise, methods for specifically targeting cells that express elevated levels of antigen (e.g., cancer cells) with CAR T-cell therapies are provided.

The present application is a continuation of U.S. Application No. 16/600,806, filed Oct. 14, 2019, which is a divisional of U.S. Application No. 15/305,996, filed Oct. 21, 2016, as a national phase application under 35 U.S.C. § 371 of International Application No. PCT/US2015/027277, filed Apr. 23, 2015, which claims the priority benefit of U.S. Provisional Application No. 61/983,103, filed Apr. 23, 2014 and U.S. Provisional Application No. 61/983,298, filed Apr. 23, 2014, the entire contents of each of which are incorporated herein by reference.

### INCORPORATION OF SEQUENCE LISTING

This application contains a Sequence Listing XML, which has been submitted electronically and is hereby incorporated by reference in its entirety. Said XML Sequence Listing, created on Jan. 11, 2023, is named UTSCP1238USC1.xml and is 21,744 bytes in size.

### BACKGROUND OF THE INVENTION

### 1. Field of the Invention

The present invention relates generally to the fields of medicine, immunology, cell biology, and molecular biology. In certain aspects, the field of the invention concerns immunotherapy. More particularly, embodiments described herein concern the production of chimeric antigen receptors (CARs) and CAR-expressing T cells that can specifically target cells with elevated expression of a target antigen.

### 2. Description of Related Art

The potency of clinical-grade T cells can be improved by combining gene therapy with immunotherapy to engineer a biologic product with the potential for superior (i) recognition of tumor-associated antigens (TAAs), (ii) persistence after infusion, (iii) potential for migration to tumor sites, and (iv) ability to recycle effector functions within the tumor microenvironment. Such a combination of gene therapy with immunotherapy can redirect the specificity of T cells for B-lineage antigens and patients with advanced B-cell malignancies benefit from infusion of such tumor-specific T cells (Jena et al., 2010; Till et al., 2008; Porter et al., 2011; Brentjens et al., 2011; Cooper et al., 2012; Kalos et al., 2011; Kochenderfer et al., 2010; Kochenderfer et al., 2012; Brentjens et al., 2013). Most approaches to genetic manipulation of T cells engineered for human application have used retrovirus and lentivirus for the stable expression of chimeric antigen receptor (CAR) (Jena et al., 2010; Ertl et al., 2011; Kohn et al., 2011). This approach, although compliant with current good manufacturing practice (cGMP), can be expensive as it relies on the manufacture and release of clinical-grade recombinant virus from a limited number of production facilities.

One draw back of CAR T-cell based therapies is the potential for off-target effects when target antigens are also expressed in normal non-diseased tissues. Accordingly, new CAR T-cell therapies are needed that provide specific targeting of diseased cells whiles reducing the side effects on normal tissues.

### SUMMARY OF THE INVENTION

Certain embodiments described herein are based on the finding that chimeric antigen receptor (CAR) T cells can be used to target cells that overexpress an antigen. Thus, in some aspects, cytotoxic activity of the CAR T cells can be focused only on intended target cells with a high level of antigen expression (e.g., cancer cells) while cytotoxic effects relative to cells having a lower level of antigen expression are minimized. In particular, it was found that by using CARs having an intermediate level of target affinity, CAR T cells could be produced that were selectively cytotoxic to cells with high antigen expression levels. Without being bound by any particular mechanism, the observed effect may be due to multivalent antigen binding by the CAR T cells to facilitate cell targeting. Alternatively or additionally, the expression level of a CAR may be adjusted in a selected CAR T cell so as reduce the off-target cytotoxicity of the cells.

Thus, in a first embodiment there are provided transgenic cells (e.g., an isolated transgenic cell) comprising an expressed CAR targeted to an antigen, said CAR having a K_{d} of between about 5 nM and about 500 nM relative to the antigen. In a further embodiment there is provided a transgenic T cell comprising an expressed CAR targeted to an antigen, said T cell exhibiting significant cytotoxic activity only upon multivalent binding of the antigen by the T cell. In an aspect, isolated cells of the embodiments are T cells or T-cell progenitors. In yet a further aspect, the cells are mammalian cells such as human cells.

In a further embodiment there are provided methods of selectively targeting cells expressing an antigen in a subject comprising (a) selecting a CAR T cell comprising an expressed CAR that binds to the antigen, said CAR T cells having: (i) cytotoxic activity only upon multivalent binding of the antigen by the T cells; and/or (ii) a CAR having a K_{d} of between about 5 nM and about 500 nM relative to the antigen; and (b) administering an effective amount of the selected CAR T cells to the subject to provide a T-cell response that selectively targets cells having elevated expression of the antigen. Thus, in certain aspects, a method of the embodiments is further defined as a method of treating a disease associated with an elevated level of antigen expression on diseased cells. For example, methods of the embodiments may be used for the treatment of a hyperproliferative disease, such as a cancer or autoimmune disease, or for the treatment of an infection, such as a viral, bacterial or parasitic infection.

In still a further embodiment there are provided methods of selectively targeting cells expressing an antigen in a mixed cell population comprising (a) selecting a CAR T cell comprising an expressed CAR that binds to the antigen, said CAR T cells having (i) cytotoxic activity only upon multivalent binding of the antigen by the T cells; and/or (ii) a CAR having a K_{d} of between about 5 nM and about 500 nM relative to the antigen; and (b) contacting a mixed cell population, said population including cells expressing different levels of the antigen, with the selected CAR T cells to selectively target cells having elevated expression of the antigen. For example, in certain aspects, a mixed cell population comprises non-cancer cells that express the antigen and cancer cells having elevated expression of the antigen. In some aspects, an elevated level of an antigen can refer to an expression level at least about: 0.5, 1, 2, 3, 4, 5, 6, 7, 8, 9, 10, 15, 20, 25, 30, 35, 40, 45, 50, 75, 100, 200, 300, 400, 500, 600, 700, 800, 900 or 1,000 times higher in a cell that is targeted by the CAR T cell.

In a further embodiment there are provided methods of selecting a CAR T cell comprising (a) obtaining a plurality of CAR T cells expressing CARs that bind to an antigen, said plurality of cells comprising (i) CARs with different affinities for the antigen (or having different on/off rates for the antigen) and/or (ii) CARs that are expressed at different levels in the cells (i.e., present at different levels on the cell surface); (b) assessing the cytotoxic activity of the cells on control cells expressing the antigen and on target cells expressing an elevated level of the antigen; and (c) selecting a CAR T cell that is selectively cytotoxic to target cells. In further aspects, methods of the embodiments further comprise expanding and/or banking a selected CAR T cell or population of selected T cells. In yet further aspects, methods of the embodiments comprise treating a subject with an effective amount of selected CAR T cells of the embodiments. In certain aspects, obtaining a plurality of CAR T cells can comprise generating a library of CAR T cells expressing CARs that bind to an antigen. For example, the library of CAR T cells may comprise random or engineered point mutations in the CAR (e.g., thereby modulating the affinity or on/off rates for the CARs). In a further aspect, a library of CAR T-cells comprises cells expressing CARs under the control of different promoter elements that provide varying levels of expression of the CARs.

In yet a further embodiment there are provided transgenic cells (e.g., an isolated transgenic cell) comprising an expressed CAR targeted to an EGFR antigen, said CAR having CDR sequences of nimotuzumab (see, e.g., SEQ ID NO: 1 and SEQ ID NO: 2) or the CDR sequences of cetuximab (see, e.g., SEQ ID NO: 3 and SEQ ID NO: 4). In some aspects, a cell of the embodiments is a human T cell comprising an expressed CAR sequence having the CDRs or the antigen binding portions of SEQ ID NO: 1 and SEQ ID NO: 2. In further aspects, a cell of the embodiments is a human T cell comprising an expressed CAR sequence having the CDRs or the antigen binding portions of SEQ ID NO: 3 and SEQ ID NO: 4.

Aspects of the embodiments concern antigens that are bound by a CAR. In some aspects, the antigen is an antigen that is elevated in cancer cells, in autoimmune cells or in cells that are infected by a virus, bacteria or parasite. In certain aspects, the antigen is CD19, CD20, ROR1, CD22, carcinoembryonic antigen, alphafetoprotein, CA-125, 5T4, MUC-1, epithelial tumor antigen, prostate-specific antigen, melanoma-associated antigen, mutated p53, mutated ras, HER2/Neu, folate binding protein, HIV-1 envelope glycoprotein gp120, HIV-1 envelope glycoprotein gp41, GD2, CD123, CD33, CD138, CD23, CD30, CD56, c-Met, mesothelin, GD3, HERV-K, IL-11Ralpha, kappa chain, lambda chain, CSPG4, ERBB2, EGFRvIII or VEGFR2. In some specific aspects the antigen is GP240, 5T4, HER1, CD-33, CD-38, VEGFR-1, VEGFR-2, CEA, FGFR3, IGFBP2, IGF-1R, BAFF-R, TACI, APRIL, Fn14, ERBB2 or ERBB3. In some further aspects, the antigen is a growth factor receptor such as EGFR, ERBB2 or ERBB3.

Certain aspects of the embodiments concern a selected CAR (or a selected cell comprising a CAR) that binds to an antigen and has a K_{d} of between about 2 nM and about 500 nM relative to the antigen. For example, in some aspects, the CAR has a K_{d} of 2, 3, 4, 5, 6, 7, 8, 9, 10, 11, 12, 13, 14, 15, 16, 17, 18, 19 or 20 nM or greater relative to the antigen. In still further aspects, the CAR has a K_{d} of between about 5 nM and about 450, 400, 350, 300, 250, 200, 150, 100 or 50 nM relative to the antigen. In still further aspects, the CAR has a K_{d} of between about 5 nM and 500 nM, 5 nM and 200 nM, 5 nM and 100 nM, or 5 nM and 50 nM relative to the antigen. As used herein reference to “K_{d} for a CAR” may refer to the K_{d} measured for a monoclonal antibody that is used to form the CAR.

In some aspects, a selected CAR of the embodiments can bind to 2, 3, 4 or more antigen molecules per CAR molecule. In some aspects, each to the antigen binding domains of a selected CAR has a K_{d} of 2, 3, 4, 5, 6, 7, 8, 9, 10, 11, 12, 13, 14, 15, 16, 17, 18, 19 or 20 nM or greater relative to the antigen. In still further aspects, each to the antigen binding domains of a selected CAR has a K_{d} of between about 5 nM and about 450, 400, 350, 300, 250, 200, 150, 100 or 50 nM relative to the antigen. In still further aspects, each to the antigen binding domains of a selected CAR has a K_{d} of between about 5 nM and 500 nM, 5 nM and 200 nM, 5 nM and 100 nM, or 5 nM and 50 nM relative to the antigen.

In some aspects of the embodiments a selected CAR for use according to the embodiments is a CAR that binds to EGFR. For example, the CAR can comprise the CDR sequences of Nimotuzumab. For example, in some aspects a CAR of the embodiments comprises all six CDRs of Nimotuzumab (provided as SEQ ID NOs: 5-10). In some aspects a CAR comprises the antigen binding portions of SEQ ID NO: 1 and SEQ ID NO: 2. In some aspects, the CAR comprises a sequence at least about 90%, 91%, 92%, 93%, 94%, 95%, 96%, 97%, 98%, 99% or 100% identical to SEQ ID NO: 1 and/or SEQ ID NO: 2. In still further aspects, a CAR for use according the embodiments does not comprise the CDR sequences of Nimotuzumab.

In a further embodiment there are provided isolated cells comprising a selected CAR and at least a second expressed transgene, such as an expressed membrane-bound IL-15. For example, in some aspects, the membrane-bound IL-15 comprises a fusion protein between IL-15 and IL-15Rα. In some cases, such a second transgene is encoded by a RNA or a DNA (e.g., an extra chromosomal or episomal vector). In certain aspects, the cell comprises DNA encoding the membrane-bound IL-15 integrated into the genome of the cell (e.g., coding DNA flanked by transposon repeat sequences). In certain aspects, a cell of the embodiments (e.g., human CAR T cell expressing a membrane-bound cytokine) can be used to treat a subject (or provide an immune response in a subject) having a disease where disease cells express elevated levels of the antigen.

In some aspects, methods of the embodiments concern transfecting T cells with a DNA (or RNA) encoding a selected CAR and, in some cases, a transposase. Methods of transfecting cells are well known in the art, but in certain aspects, highly efficient transfection methods such as electroporation or viral transduction are employed. For example, nucleic acids may be introduced into cells using a nucleofection apparatus. Preferably, however, the transfection step does not involve infecting or transducing the cells with a virus, which can cause genotoxicity and/or lead to an immune response to cells containing viral sequences in a treated subject.

Certain aspects of the embodiments concern transfecting cells with an expression vector encoding a selected CAR. A wide range of expression vectors for CARs are known in the art and are further detailed herein. For example, in some aspects, the CAR expression vector is a DNA expression vector such as a plasmid, linear expression vector or an episome. In certain aspects, the vector comprises additional sequences, such as sequences that facilitate expression of the CAR, such as a promoter, enhancer, poly-A signal, and/or one or more introns. In preferred aspects, the CAR coding sequence is flanked by transposon sequences, such that the presence of a transposase allows the coding sequence to integrate into the genome of the transfected cell.

As detailed supra, in certain aspects, cells are further transfected with a transposase that facilitates integration of a CAR coding sequence into the genome of the transfected cells. In some aspects, the transposase is provided as a DNA expression vector. However, in preferred aspects, the transposase is provided as an expressible RNA or a protein such that long-term expression of the transposase does not occur in the transgenic cells. Any transposase system may be used in accordance with the embodiments. However, in some aspects, the transposase is salmonid-type Tcl-like transposase (SB). For example, the transposase can be the “Sleeping beauty” transposase, see, e.g., U.S. Pat.6,489,458, incorporated herein by reference.

In still further aspects, a selected CAR T cell of the embodiments further comprises an expression vector for expression of a membrane-bound cytokine that stimulates proliferation of T cells. In particular, selected CAR T cells comprising such cytokines can proliferate with little or no ex vivo culture with antigen presenting cells due the simulation provided by the cytokine expression. Likewise, such CAR T cells can proliferate in vivo even when large amounts of antigen recognized by the CAR is not present (e.g., as in the case of a cancer patient in remission or a patient with minimal residual disease). In some aspects, the CAR T cells comprise a DNA or RNA expression vector for expression of a Cγ cytokine and elements (e.g., a transmembrane domain) to provide surface expression of the cytokine. For example, the CAR cells can comprise membrane-bound versions of IL-7, IL-15 or IL-21. In some aspects, the cytokine is tethered to the membrane by fusion of the cytokine coding sequence with the receptor for the cytokine. For example, a cell can comprise a vector for expression of an IL-15-IL-15Rα fusion protein. In still further aspects, a vector encoding a membrane-bound Cγ cytokine is a DNA expression vector, such as a vector integrated into the genome of the CAR cells or an extra-chromosomal vector (e.g., and episomal vector). In still further aspects, expression of the membrane-bound Cγ cytokine is under the control of an inducible promoter (e.g., a drug inducible promoter) such that the expression of the cytokine in the CAR cells (and thereby the proliferation of the CAR cells) can be controlled by inducing or suppressing promoter activity.

Aspects of the embodiments concern obtaining T cells or T-cell progenitors for expression of selected CARs. In some aspects, the cells are obtained from a third party, such as a tissue bank. In further aspects, cell samples from a patient comprising T cells or T-cell progenitors are used. For example, in some cases, the sample is an umbilical cord blood sample, a peripheral blood sample (e.g., a mononuclear cell fraction) or a sample from the subject comprising pluripotent cells. In some aspects, a sample from the subject can be cultured to generate induced pluripotent stem (iPS) cells and these cells used to produce T cells. Cell samples may be cultured directly from the subject or may be cryopreserved prior to use. In some aspects, obtaining a cell sample comprises collecting a cell sample. In other aspects, the sample is obtained by a third party. In still further aspects, a sample from a subject can be treated to purify or enrich the T cells or T-cell progenitors in the sample. For example, the sample can be subjected to gradient purification, cell culture selection and/or cell sorting (e.g., via fluorescence-activated cell sorting (FACS)).

In some aspects, a method of the embodiments further comprises obtaining, producing or using antigen presenting cells (APCs). For example, in some aspects, the antigen presenting cells comprise dendritic cells, such as dendritic cells that express or have been loaded with and an antigen of interest. In further aspects, the antigen presenting cell can comprise artificial antigen presenting cells that display an antigen of interest. For example, artificial antigen presenting cells can be inactivated (e.g., irradiated) artificial antigen presenting cells (aAPCs). Methods for producing such aAPCs are know in the art and further detailed herein.

Thus, in some aspects, transgenic CAR cells of the embodiments are co-cultured with antigen presenting cells (e.g., inactivated aAPCs) ex vivo for a limited period of time in order to expand the CAR cell population. The step of co-culturing CAR cells with antigen presenting cells can be done in a medium that comprises, for example, interleukin-21 (IL-21) and/or interleukin-2 (IL-2). In some aspects, the co-culturing is performed at a ratio of CAR cells to APCs of about 10:1 to about 1:10; about 3:1 to about 1:5; or about 1:1 to about 1:3. For example, the co-culture of CAR cells and APCs can be at a ratio of about 1:1, about 1:2 or about 1:3.

In some aspects, APCs for culture of selected CAR cells are engineered to express a specific polypeptide to enhance growth of the CAR cells. For example, the APCs can comprise (i) an antigen targeted by the CAR expressed on the transgenic CAR cells; (ii) CD64; (ii) CD86; (iii) CD137L; and/or (v) membrane-bound IL-15, expressed on the surface of the APCs. In some aspects, the APCs comprise a CAR-binding antibody or fragment thereof expressed on the surface of the APCs (see, e.g., International PCT pat. publication WO/2014/190273, incorporated herein by reference). Preferably, APCs for use in the instant methods are tested for, and confirmed to be free of, infectious material and/or are tested and confirmed to be inactivated and non-proliferating.

While expansion on APCs can increase the number or concentration of CAR cells in a culture, this proceed is labor intensive and expensive. Moreover, in some aspects, a subject in need of therapy should be re-infused with transgenic CAR cells in as short a time as possible. Thus, in some aspects, ex vivo culturing of selected CAR cells is for no more than 14 days, no more than 7 days or no more than 3 days. For example, the ex vivo culture (e.g., culture in the presence of APCs) can be performed for less than one population doubling of the transgenic CAR cells. In still further aspects, the transgenic cells are not cultured ex vivo in the presence of APCs.

In still further aspects, a method of the embodiments comprises a step for enriching the cell population for selected CAR-expressing T cells before administering or contacting the cells to a population (e.g., after transfection of the cells or after ex vivo expansion of the cells). For example, the enrichment step can comprise sorting of the cell (e.g., via Fluorescence-activated cell sorting (FACS)), for example, by using an antigen bound by the CAR or a CAR-binding antibody. In still further aspects, the enrichment step comprises depletion of the non-T cells or depletion of cells that lack CAR expression. For example, CD56⁺ cells can be depleted from a culture population. In yet further aspects, a sample of CAR cells is preserved (or maintained in culture) when the cells are administered to the subject. For example, a sample may be cryopreserved for later expansion or analysis.

In certain aspects, transgenic CAR cells of the embodiments are inactivated for expression of an endogenous T-cell receptor and/or endogenous HLA. For example, T cells can be engineered to eliminate expression of endogenous alpha/beta T-cell receptor (TCR). In specific embodiments, CAR⁺ T cells are genetically modified to eliminate expression of TCR. In some aspects, there is a disruption of the endogenous T-cell receptor in CAR-expressing T cells. For example, in some cases an endogenous TCR (e.g., a α/β or γ/δ TCR) is deleted or inactivated using a zinc finger nuclease (ZFN) or CRISPR/Cas9 system. In certain aspects, the T-cell receptor αβ-chain in CAR-expressing T cells is knocked-out, for example, by using zinc finger nucleases.

As further detailed herein, CAR cells of the embodiments can be used to treat a wide range of diseases and conditions. Essentially any disease that involves the enhanced expression of a particular antigen can be treated by targeting CAR cells to the antigen. For example, autoimmune diseases, infections, and cancers can be treated with methods and/or compositions of the embodiments. These include cancers, such as primary, metastatic, recurrent, sensitive-to-therapy, refractory-to-therapy cancers (e.g., chemo-refractory cancer). The cancer may be of the blood, lung, brain, colon, prostate, breast, liver, kidney, stomach, cervix, ovary, testes, pituitary gland, esophagus, spleen, skin, bone, and so forth (e.g., B-cell lymphomas or a melanomas). In certain aspects, a method of the embodiments is further defined as a method of treating a glioma, such as a diffuse intrinsic pontine glioma. In the case of cancer treatment, CAR cells typically target a cancer cell antigen (also known as a tumor-associated antigen (TAA)), such as EGFR.

The processes of the embodiments can be utilized to manufacture (e.g., for clinical trials) CAR⁺ T cells for various tumor antigens (e.g., CD19, ROR1, CD56, EGFR, CD123, c-met, GD2). CAR⁺ T cells generated using this technology can be used to treat patients with leukemias (AML, ALL, CML), infections and/or solid tumors. For example, methods of the embodiments can be used to treat cell proliferative diseases, fungal, viral, bacterial or parasitic infections. Pathogens that may be targeted include, without limitation, Plasmodium, trypanosome, Aspergillus, Candida, HSV, RSV, EBV, CMV, JC virus, BK virus, or Ebola pathogens. Further examples of antigens that can be targeted by CAR cells of the embodiments include, without limitation, CD19, CD20, carcinoembryonic antigen, alphafetoprotein, CA-125, 5T4, MUC-1, epithelial tumor antigen, melanoma-associated antigen, mutated p53, mutated ras, HER2/Neu, ERBB2, folate binding protein, HIV-1 envelope glycoprotein gp120, HIV-1 envelope glycoprotein gp41, GD2, CD123, CD23,CD30, CD56, c-Met, meothelin, GD3, HERV-K, IL-11Ralpha, kappa chain, lambda chain, CSPG4, ERBB2, EGFRvIII, or VEGFR2. In certain aspects, method of the embodiments concern targeting of CD19 or HERV-K-expressing cells. For example, a HERV-K targeted CAR cell can comprise a CAR including the scFv sequence of monoclonal antibody 6H5. In still further aspects, a CAR of the embodiments can be conjugated or fused with a cytokine, such as IL-2, IL-7, IL-15, IL-21 or a combination thereof.

In some embodiments, methods are provided for treating an individual with a medical condition comprising the step of providing an effective amount of cells from a population of CAR expressing T cells or T-cell progenitors (e.g., CAR expressing T-cells that selectively kill cells that have an elevated expression level of a target antigen) to the subject. In some aspects, the cells can be administered to an individual more than once (e.g., 2, 3, 4, 5 or more times). In further aspects, cells are administered to an individual at least 1, 2, 3, 4, 5, 6, 7, 8, 9, 10, 11, 12, 13, 14 or more days apart. In specific embodiments, the individual has a cancer, such a lymphoma, leukemia, non-Hodgkin’s lymphoma, acute lymphoblastic leukemia, chronic lymphoblastic leukemia, chronic lymphocytic leukemia, or B cell-associated autoimmune diseases.

In a further embodiment, there is provided an isolated transgenic cell (e.g., a T-cell or T-cell progenitor) comprising an expressed CAR targeted to EGFR. For example, the CAR can comprise the CDR sequences of Nimotuzumab. For example, in some aspects, a cell of the embodiments comprises a CAR comprising all six CDRs of Nimotuzumab (provided as SEQ ID NOs: 5-10). In some aspects, the CAR comprises the antigen binding portions of SEQ ID NO: 1 and SEQ ID NO: 2. In further aspects, the CAR comprises a sequence at least about 90%, 91%, 92%, 93%, 94%, 95%, 96%, 97%, 98%, 99% or 100% identical to SEQ ID NO: 1 and/or SEQ ID NO: 2. In still further aspects, a cell of the embodiments comprises a CAR that does not comprise the CDR sequences of Nimotuzumab. In some aspects, there is provided a pharmaceutical composition comprising an isolated transgenic cell of the embodiments. In a further related embodiment there is provided a method of treating a subject having an EGFR positive cancer comprising administering an effective amount of transgenic human T-cells to the subject said T-cells comprising an expressed CAR targeted to EGFR and comprising the CDR sequences of SEQ ID NOs: 5-10.

In a further embodiment, there is provided an isolated transgenic cell (e.g., a T-cell or T-cell progenitor) comprising an expressed CAR that comprises the CDR sequences of Cetuximab. For example, in some aspects, a cell of the embodiments comprises a CAR comprising all six CDRs of Cetuximab (provided as SEQ ID NOs: 11-16). In some aspects, the CAR comprises the antigen binding portions of SEQ ID NO: 3 and SEQ ID NO: 4. In further aspects, the CAR comprises a sequence at least about 90%, 91%, 92%, 93%, 94%, 95%, 96%, 97%, 98%, 99% or 100% identical to SEQ ID NO: 3 and/or SEQ ID NO: 4. In still further aspects, a cell of the embodiments comprises a CAR that does not comprise the CDR sequences of Cetuximab. In some aspects, there is provided a pharmaceutical composition comprising an isolated transgenic cell of the embodiments. In a further related embodiment there is provided a method of treating a subject having an EGFR positive cancer comprising administering an effective amount of transgenic human T-cells to the subject said T-cells comprising an expressed CAR targeted to EGFR and comprising the CDR sequences of SEQ ID NOs: 11-16.

As used herein in the specification and claims, “a” or “an” may mean one or more. As used herein in the specification and claims, when used in conjunction with the word “comprising”, the words “a” or “an” may mean one or more than one. As used herein, in the specification and claim, “another” or “a further” may mean at least a second or more.

As used herein in the specification and claims, the term “about” is used to indicate that a value includes the inherent variation of error for the device, the method being employed to determine the value, or the variation that exists among the study subjects.

Other objects, features and advantages of the present invention will become apparent from the following detailed description. It should be understood, however, that the detailed description and the specific examples, are given by way of illustration only, since various changes and modifications within the spirit and scope of the invention will become apparent to those skilled in the art from this detailed description.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIGS. 1A-B. Numeric expansion of human primary T cells with artificial antigen presenting cells loaded with anti-CD3. (A) Phenotype of K562 clone 4 loaded to express anti-CD3 (OKT3) and irradiated to 100 gray measured by flow cytometry. (B) Numeric expansion of CD3⁺ T cells following stimulation with low density of OKT3-loaded aAPC (10 T cells to 1 aAPC) or high density of OKT3-loaded K562 (1 T cell to 2 aAPC). Inferred cell count calculated by multiplying fold expansion following a stimulation cycle to the total number of T cells prior to stimulation cycle. Data represented as mean ± SD, n=6, **** p<0.0001, two-way ANOVA (Tukey’s post-test).

FIGS. 2A-D. T cells expanded on low density aAPC contain higher ratio of CD8⁺ T cells. (A) T cells expanded with low density aAPC (10 T cells to 1 aAPC) contain significantly more CD8⁺ T cells and significantly less CD4⁺ T cells than T cells expanded with high density aAPC (1 T cell to 2 aAPC) as measured by flow cytometry following two stimulation cycles. Data represented as mean, n=6, *** p<0.001, **** p<0.0001, two-way ANOVA (Tukey’s post-test). (B) Differences in CD4/CD8 ratio in T cells expanded with low density aAPC and high density aAPC is due to reduced fold expansion of CD4⁺ T cells when expanded with low density aAPC. Data represented as mean ± SD, n=6, **** p<0.0001, two-way ANOVA (Tukey’s post-test). (C) Differences in CD4/CD8 ratio in T cells expanded woth low density aAPC and high density aAPC is not due to differences in cell viability. Viability of cells was determined by flow cytometry for Annexin V and PI staining following two stimulation cycles where Annexin V^{neg} PI^{neg} cells are considered live cells. Data represented as mean ± SD, n=3. (D) CD4⁺ T cells have less proliferation when stimulation was low density aAPC than high density aAPC. Ki-67 was measured by intracellular flow cytometry as a marker for cellular proliferation following two stimulation cycles. Representative histograms from three independent donors shown.

FIG. 3. Differential gene expression in T cell stimulated with low or high density aAPC. Differential gene expression between CD4⁺ and CD8⁺ T cells stimulated with low or high density aAPC measured by multiplexed digital profiling of mRNA species following two cycles of stimulation. Significant up- or down-regulated transcripts was determined by greater than 1.5 fold difference in transcript level in ⅔ donors and p<0.01. Data represented by heat-map of fold difference, n=3.

FIGS. 4A-C. T cells expanded with low density aAPC have more central-memory phenotype T cells. (A) Memory marker analysis of T cells expanded with low density or high density aAPC was measured by flow cytometry for CCR7 and CD45RA following two cycles of stimulation. Cell populations in gated CD4⁺ and CD8⁺ T cell populations were defined as follows: effector memory = CCR7^{neg}CD45RA^{neg}, central memory = CCR7⁺CD45RA^{neg}, naïve = CCR7⁺CD45RA⁺, effector memory RA = CCR7^{neg}CD45RA⁺. Data represented as mean ± SD, n=3,* p<0.05, two-way ANOVA (Tukey’s post-test). (B) Intracellular staining for granzyme and perforin in T cells following two stimulation cycles was measured by flow cytometry in CD4⁺ and CD8⁺ gated T cell populations. Data represented as mean ± SD, n=3, *p<0.05, *** p<0.001, two-way ANOVA (Tukey’s post-test). (C) Cytokine production following stimulation with PMA/Ionomycin was measured by intracellular cytokine staining in T cells following two cycles of stimulations by flow cytometry in CD4⁺ and CD8⁺ gated T cell populations. Data represented as mean ± SD, n=3, *p<0.05, *** p<0.001, two-way ANOVA (Tukey’s post-test).

FIG. 5. Diversity of TCR Vα after numeric expansion of T cells on aAPC. Diversity of TCR Vα in T cells expanded with low or high density aAPC was measured by digital multiplexed profiling of mRNA species and relative abundance of each TCR Vα was calculated as percent of total TCR Vα transcripts. Data represented as mean ± SD, n=3.

FIG. 6. Diversity of TCR Vβ after numeric expansion of T cells on aAPC. Diversity of TCR Vβ in T cells expanded with low or high density aAPC was measured in sorted CD4⁺ and CD8⁺ T cells by digital multiplexed profiling of mRNA species and relative abundance of each TCR Vα was calculated as percent of total TCR Vα transcripts. Data represented as mean ± SD, n=3.

FIG. 7. Diversity of CDR3 sequences after numeric expansion on aAPC. CDR3 sequences of TCR Vβ chain were determined by high-throughput sequences on ImmunoSEQ platform. Numbers of each unique sequence before numeric expansion were plotted against the numbers of the same sequence after numeric expansion with low density (10 T cells to 1 aAPC) or high density (1 T cell to 2 aAPC) aAPC. Data were fit with a linear regression and slope was determined. Data representative of two individual donors.

FIGS. 8A-D. Optimization of RNA transfer to T cells numerically expanded with aAPC. (A) Expression of GFP RNA and viability of T cells electroporated with various programs after expansion with aAPC. Median fluorescence intensity of GFP was determined by flow cytometry. Viability was determined by PI stain and flow cytometry. Data representative of two individual donors. (B) Expression of GFP RNA and viability in T cells expanded with aAPC at low density (10 T cells to 1 aAPC) following one, two or three cycles of stimulation. Percentage of T cells expressing GFP was determined by flow cytometry. Viability was determined by PI stain and flow cytometry. Data representative of two individual donors. (C) Expression of GFP RNA and viability of T cells stimulated at an aAPC density of 10 T cells to 1 aAPC for two stimulation cycles after electroporation with various programs. Percentage of T cells expressing GFP was determined by flow cytometry. Viability was determined by PI stain and flow cytometry. Data representative of two individual donors. (D) Expression of memory markers CCR7 and CD45RA measured by flow cytometry in CD4⁺ and CD8⁺ gated T cells following two cycles of stimulation with aAPC at a density of 10 T cells to 1 aAPC, mock electroporated with no RNA, and electroporated with RNA. Data represented as mean ± SD, n=3.

FIGS. 9A-B. Schematic of CAR expression by DNA and RNA modification. (A) DNA modification of T cells by electroporation with SB transposon/transposase. Normal donor PBMCs are electroporated with SB transposon containing CAR and SB11 transposase to result in stable CAR expression in a fraction of T cells. Stimulation with γ-irradiated antigen expressing aAPC in the presence of IL-21 (30 ng/mL) and IL-2 (50 U/mL) cull out CAR⁺ T cells over time, resulting in >85% CAR⁺ T cells following 5 stimulation cycles and T cells are evaluated for CAR-mediated function. (B) Modification of T cells by RNA electro-transfer. Normal donor PBMCs are stimulated with γ-irradiated anti-CD3 (OKT3) loaded K562 clone 4 aAPC. Three to five days following second stimulation, T cells are electroporated with RNA to result in >95% CAR⁺ T cells 24 hours after RNA electro-transfer, and T cells are evaluated for CAR-mediated function.

FIGS. 10A-E. Phenotype of Cetux-CAR⁺ T cells modified by DNA and RNA. (A) Median fluorescence intensity of CAR expression in RNA-modified and DNA-modified T cells determined by flow cytometry for IgG region of CAR in CD4⁺ and CD8⁺ gated T-cell populations. Data represented as mean ± SD, n=8. (B) Proportion of CD4⁺ and CD8⁺ T-cell populations in RNA- and DNA-modified T cells determined by flow cytometry for CD4 and CD8 in CAR⁺ gated T cells. Data represented as mean ± SD, n=8. (C) Expression of memory markers CCR7 and CD45RA determined by flow cytometry in CD4⁺ and CD8⁺ gated T-cell populations. Memory populations were defined as follows: effector memory = CCR7″°⁸CD45RA″°⁸, central memory = CCR7⁺CD45RA^{neg}, naïve = CCR7⁺CD45RA⁺, effector memory RA = CCR7^{neg}CD45RA⁺. Data represented as mean ± SD, n=3, **** p<0.0001, two-way ANOVA (Tukey’s post-test). (D) Expression of inhibitory receptor PD-1 and marker of replicative senescence CD57 as determined in CD4⁺ and CD8⁺ gated T-cell populations by flow cytometry. Data represented as mean ± SD, n=3, ** p<0.01, two-way ANOVA (Tukey’s post-test). (E) Expression of granzyme B and perforin determined by intracellular cytokine staining in CD4⁺ and CD8⁺ gated T-cell populations by flow cytometry. Data represented as mean ± SD, n=3.

FIGS. 11A-C. DNA-modified CAR⁺ T cells produce more cytokine and display slightly more cytotoxicity than RNA-modified CAR⁺ T cells. (A) Cytokine production of DNA-modified (following 5 stimulation cycles) and RNA-modified CAR⁺ T cells (24 hours post RNA transfer) was measured by intracellular staining and flow cytometry following 4 hr incubation with targets or PMA/Ionomycin in CD8⁺ gated T cells. Data represented as mean ± SD, n=3, * p<0.05, ** p<0.01, *** p<0.001, **** p<0.0001, two-way ANOVA (Tukey’s post-test). (B) Specific cytotoxicity of DNA-modified (following 5 stimulation cycles) and RNA-modiifed CAR⁺ ¬T cells (24 hours post RNA transfer) was determined by standard 4 \-hour chromium release assay. Data represented as mean ± SD, n=3, *p<0.05, two-way ANOVA (Tukey’s post-test). (C) Specific cytotoxicity of A431 by RNA-modified CAR⁺ T cells at 10:1 effector:target ratio plotted against median fluorescence intensity of CAR. Linear regression was fit to the data, yielding a slope of slope=0.0237±0.030, not significantly different from a slope of 0, p=0.4798.

FIGS. 12A-C. Transient expression of Cetux-CAR by RNA-modification of T cells. (A) Expression of CAR measured daily by flow cytometry for IgG portion of CAR with no cytokines or stimulus added to T cells. Data representative of three independent donors. (B) Expression of CAR measured daily by flow cytometry for IgG portion of CAR following addition of IL-2 (50 U/mL) and IL-21 (30 ng/mL) 24 hours after RNA transfer. Data representative of three independent donors. (C) Expression of CAR measured daily by flow cytometry for IgG portion of CAR after addition of tEGFR⁺ EL4 cells 24 hours after RNA transfer. Data representative of three independent donors.

FIGS. 13A-C. Transient expression of Cetux-CAR by RNA modification reduces cytokine production and cytotoxicity to EGFR-expressing cells. (A) Production of IFN-γ measured by intracellular staining and flow cytometry in DNA-modified and RNA-modified CD8⁺ T cells 24 hours and 120 hours after RNA transfer after 4 hour incubation with target cells or PMA/Ionomycin. Data represented as mean ± SD, n=3, * p<0.05, two-way ANOVA (Tukey’s post-test). (B) Specific cytotoxicity of DNA-modified and RNA-modified T cells measured by standard chromium release assay 24 hours and 120 hours after RNA transfer. Data represented as mean ± SD, n=3, * p<0.05, ** p<0.01, **** p<0.0001, two-way ANOVA (Tukey’s post-test). (C) Change in specific cytotoxicity of DNA-modified and RNA-modified T cells from 24 hours post RNA transfer to 120 hours post RNA transfer measured by standard chromium release assay at an effector to target ratio of 10:1. Data represented as mean ± SD, n=3, * p<0.05, two-way ANOVA (Tukey’s post-test).

FIGS. 14A-D. Numeric expansion of Cetux-CAR⁺ and Nimo-CAR⁺ T cells. (A) Phenotype of γ-irradiated tEGFR⁺ K562 clone 27 determined by flow cytometry. (B) Numeric expansion of Cetux-CAR⁺ and Nimo-CAR⁺ T cells. Prior to each stimulation cycle, percentage of CD3⁺CAR⁺ T cells was determined by flow cytometry. Inferred cell count was calculated by multiplying the fold expansion following a stimulation cycle by the number of CAR⁺ T cells stimulated. Data represented as mean ± SD, n=7. (C) Expression of CAR in CD3⁺ T cells was determined 24 hours after electroporation of CAR and after 28 days of expansion by flow cytometry for the IgG portion of CAR. Data represented as mean, n=7. (D) Median fluorescence intensity of CAR expression was determined by flow cytometry for the IgG portion of CAR after 28 days of expansion. Data represented as mean ± SD, n=7.

FIGS. 15A-C. Cetux-CAR⁺ and Nimo-CAR⁺ T cells are phenotypically similar. (A) Proportion of CD4 and CD8 T cells in total T-cell population after 28 days of expansion measured by flow cytometry on gated CD3⁺CAR⁺ cells. Data represented as mean ± SD, n=7. (B,C) Expression of T-cell memory and differentiation markers after 28 days of T-cell expansion measured by flow cytometry in gated CD4⁺ and CD8⁺ T-cell populations. Data represented as mean ± SD, n=4.

FIGS. 16A-F. Cetux-CAR⁺ and Nimo-CAR⁺ T cells are activated equivalently through affinity-independent triggering of CAR. (A) Production of IFN-γ in response to EGFR⁺ A431 in the presence of EGFR blocking monoclonal antibody. CAR⁺ T cells were co-cultured with A431 with anti-EGFR blocking antibody or isotype control and IFN-γ production was measured by intracellular flow cytometry. Percent of production was calculated as mean fluorescence intensity of IFN-γ in gated CD8⁺ T cells relative to unblocked CD8⁺ T cell production. Data represented as mean ± SD, n=3, *** p<0.001, two-way ANOVA (Tukey’s post-test). (B) Representative histograms of expression of tEGFR (top panel) and CAR-L (bottom panel) on EL4 cells relative to cell lines negative for antigen. Density of EGFR expression was determined by quantitative flow cytometry. (C) Production of IFN-γ by gated CD8⁺CAR⁺ T cells after co-culture with CD19⁺, tEGFR⁺, or CARL⁺ EL4 cells measured by intracellular staining and flow cytometry. Data represented as mean ± SD, n=4, ** p<0.01, two-way ANOVA (Tukey’s post-test). (D) Phosphorylation of p38 and Erk1/2 by phosflow cytometry in gated CD8⁺ CAR⁺ T cells 30 minutes after co-culture with CD19⁺, EGFR⁺, or CARL⁺ EL4 cells. Data represented as mean ± SD, n=2, * p<0.05, two-way ANOVA (Tukey’s post-test). (E) Specific lysis of CD19⁺, EGFR⁺ and CARL⁺ EL4 cells measured by standard 4 hour chromium release assay. Data represented as mean ± SD, n=4, **** p<0.0001, two-way ANOVA (Tukey’s post-test). (F) Relative proportion of T cells to EL4 cells in long term co-culture. Fraction of co-culture containing T cells to EL4 cells measured by flow cytometry for human and murine CD3, respectively, with non-species cross reactive antibodies. Data represented as mean ± SD, n=4, ** p<0.01, two-way ANOVA (Tukey’s post-test).

FIGS. 17A-C. Activation and functional response of Nimo CAR T cells is impacted by density of EGFR expression. A) Representative histograms of EGFR expression on A431, T98G, LN18, U87 and NALM-6 cell lines measured by flow cytometry. Number of molecules per cell determined by quantitative flow cytometry. Data representative of three replicates. B) Production of IFN-γ by CD8⁺CAR⁺ T cells in response to co-culture with A431, T98G, LN18, U87 and NALM-6 cell lines measured by intracellular flow cytometry gated on CD8⁺ cells. Data represented as mean ± SD, n=4, *** p<0.001, two-way ANOVA (Tukey’s post-test) C. Specific lysis of A431, T98G, LN18, U87 and NALM-6 by CAR⁺ T cells measured by standard 4 hour chromium release assay. Data represented as mean ± SD, n=4, **** p<0.0001, ** p<0.01, * p<0.05, two-way ANOVA (Tukey’s post-test).

FIGS. 18A-E. Activation of function of Nimo-CAR⁺ T cells is directly and positively correlated with EGFR expression density. (A) Representative histogram of EGFR expression on series of four U87-derived tumor cell lines (U87, U87low, U87med, and U87high) measured by flow cytometry. Number of molecules per cell determined quantitative flow cytometry. Data representative of triplicate experiments. (B) Phosphorylation of Erk1/2 and p38 in gated CD8⁺ T cells following co-culture with U87 or U87high for 5, 45, and 120 minutes measured by phosflow cytometry. Data represented as mean fluorescence intensity ± SD, n=2. (C) Phosphorylation of Erk1/2 and p38 MAP kinase family members in gated CD8⁺ T cells after 45 minutes of co-culture with U87 cell lines with increasing levels of EGFR measured by phosflow cytometry. Data represented as mean fluorescence intensity ± SD, n=4, **** p<0.0001, *** p<0.001, ** p<0.01, two-way ANOVA (Tukey’s post-test). (D) Production of IFN-γ and TNF-α by gated CD8⁺ CAR⁺ T cells in response to co-culture with U87 cell lines with increasing levels of EGFR measured by intracellular staining and flow cytometry. Data represented as mean ± SD, n=4, **** p<0.0001, *** p<0.001, ** p<0.01, two-way ANOVA (Tukey’s post-test). (E) Specific lysis of U87 cell lines with increasing levels of EGFR by CAR⁺ T cells measured by standard 4 hour chromium release assay. Data represented as mean ± SD, n=5, **** p<0.0001, ** p<0.01, * p<0.05, two-way ANOVA (Tukey’s post-test).

FIGS. 19A-B. Increasing interaction time does not restore Nimo-CAR⁺ T-cell function in response to low EGFR density. (A) Production of IFN-γ was measured by intracellular staining and flow cytometry following stimulation with U87 or U87high following different incubation periods in CD8⁺ gated cells. Data represented as mean ± SD, n=3. (B) Fraction of U87 and U87high cells remaining after co-culture with Cetux-CAR⁺ or Nimo-CAR⁺ T cells. U87 cell lines were co-cultured with CAR⁺ T cells at an E:T ratio of 1:5 in triplicate. Suspension T cells were separated from adherent target cells, and adherent fraction was counted by trypan blue exclusion. Percent surviving was calculated as [cell number harvested after co-culture]/[cell number without T cells]*100. Data represented as mean ± SD, n=3, *** p<0.001, two-way ANOVA (Tukey’s post-test)

FIGS. 20A-B. Increasing CAR density on T-cell surface does not restore sensitivity of Nimo-CAR⁺ T cells to low density EGFR. A) Representative histograms of CAR expression in T cells modified by RNA transfer and traditional DNA electroporation via SB system. Data representative of 2 independent experiments. B) Production of IFN-γ in T cells overexpressing CAR by RNA electro-transfer in response to low and high antigen density. Production of IFN-γ was measured by intracellular flow cytometry in CD8⁺ gated cells following stimulation with U87 or U87high target cells. Data represented as mean ± SD, n=2.

FIGS. 21A-C. Nimo-CAR⁺ T cells have less activity in response to basal EGFR levels on normal renal epithelial cells than Cetux-CAR⁺ T cells. (A) Representative histogram of expression of EGFR on HRCE measured by flow cytometry. Number of molecules per cell determined by quantitative flow cytometry. Data representative of three replicates. (B) Production of IFN-γ and TNF-α by CD8⁺CAR⁺ T cells in response to co-culture with HRCE measured by intracellular staining and flow cytometry gated on CD8⁺ cells. Data represented as mean ± SD, n=4, ** p<0.01, * p<0.05, two-way ANOVA (Tukey’s post-test). (C) Specific lysis of HRCE by CAR⁺ T cells measured by standard 4 hour chromium release assay. Data represented as mean ± SD, n=3, **** p<0.0001, *** p<0.001, two-way ANOVA (Tukey’s post-test).

FIGS. 22A-B. Cetux-CAR⁺ T cells proliferate less following stimulation than Nimo-CAR⁺ T cells, but do not have increased propensity for AICD. (A) Proliferation of CD8⁺CAR⁺ T cells after stimulation with U87 or U87high measured by intracellular flow cytometry for Ki-67 gated on CD8⁺ cells. Data represented as mean fluorescence intensity ± SD, n=4, ** p<0.01, two-way ANOVA (Tukey’s post-test). (B) Viability of T cells after stimulation with U87 or U87high measured by flow cytometry for Annexin V and 7-AAD gated on CD8⁺ cells. Percent live cells determined by percent Annevin V^{neg} 7-AAD^{neg}. Data represented as mean ± SD, n=4, *** p<0.001, two-way ANOVA (Tukey’s post-test).

FIGS. 23A-C. Cetux-CAR⁺ T cells demonstrate enhanced downregulation of CAR. (A) Surface expression of CAR during co-culture (E:T 1:5) with U87 or U87high measured by flow cytometry for IgG portion of CAR. Percent CAR remaining calculated as [%CAR⁺ in co-culture] / [%CAR⁺ in unstimulated culture] x 100. Data represented as mean ± SD, n=3, ** p<0.01. * p<0.05, two-way ANOVA (Tukey’s post-test) (B) Representative histograms of Intracellular and surface expression of CAR determined by flow cytometry after 24 hours of co-culture with U87 or U87high in CD8⁺ gated T cells. Data representative of three independent donors. (C) Surface expression of CAR during co-culture (E:T 1:1) with EGFR⁺ EL4 or CAR-L⁺ EL4 measured by flow cytometry for Fc portion of CAR. Percent CAR remaining calculated as [%CAR⁺ in co-culture] / [%CAR⁺ in unstimulated culture] x 100. Data represented as mean, n=2, * p<0.05, two-way ANOVA (Tukey’s post-test).

FIG. 24. Cetux-CAR⁺ T cells have reduced response to re-challenge with antigen. After a 24-hour incubation with U87 or U87high, CAR⁺ T cells were rechallenged with U87 or U87high and production of IFN-γ CAR⁺ T cells measured by intracellular staining and flow cytometry gated on CD8⁺ cells. Data represented as mean ± SD, n=3, *** p<0.001, ** p<0.01, * p<0.05, two-way ANOVA (Tukey’s post-test).

FIG. 25. Schematic of animal model and treatment schedule. (A) Schematic of guide screw placement. A 1-mm hole is drilled for insertion of guide screw in the right frontal lobe, 1 mm from the coronal suture and 2.5 mm from the sagittal suture. (B) Timeline of treatment schedule. Guide-screw is implanted into the right frontal lobe of mice no less than 14 days prior to injection of tumor, which is designated as day 0 of study. Tumor was imaged by BLI one day prior to initiation of T-cell treatment. CAR⁺ T cells were administered intracranially through the guide-screw weekly for three weeks. Tumor growth was assessed by BLI the prior to and following T-cell treatment while mice were actively receiving treatments, then weekly throughout remainder of experiment.

FIGS. 26A-C. Engraftment of U87med and CAR⁺ T-cell phenotype prior to T-cell treatment. (A) Four days after tumor injection, tumors were imaged by BLI following injection with D-luciferin and 10 minute incubation. (B) Mice were divided into three groups to evenly distribute relative tumor burden as determined by day 4 BLI flux measurements. (C) Cetux-CAR⁺ and Nimo-CAR⁺ T cells expanded through 4 stimulation cycles were evaluated for CAR expression and CD4/CD8 ratio by flow cytometry.

FIGS. 27A-B. Cetux-CAR⁺ and Nimo-CAR⁺ T cells inhibit growth of U87med intracranial xenografts. (A) Serial BLI assessed relative size of tumor. (B) Relative tumor growth as assessed by serial BLI of tumor. Background luminescence (gray shading) was defined by BLI of mice with no tumors. Significant difference in BLI between mice with no treatment vs. treatment (n=7) with Cetux-CAR⁺ T cells (n=7, p<0.01) and no treatment (n=7) vs. treatment with Nimo-CAR⁺ T cells (n=7, p<0.05) at day 18, two-way ANOVA (Sidak’s post-test).

FIGS. 28A-B. Survival of mice bearing U87med intracranial xenografts treated with Cetux-CAR⁺ and Nimo-CAR⁺ T cells. (A) Survival of mice with U87med-ffLuc-mKate intracranial xenografts from two independent experiments within 7 days of T-cell treatment. Significant reduction in survival in Cetux-CAR⁺ T cell treated mice 8/14 surviving) relative to untreated mice (14/14 surviving) determined by Mantel-Cox log-rank test, p=0.0006. (B) Survival of mice with U87med-ffLuc-mKate intracranial xenografts receiving no treatment, Cetux-CAR⁺ T cells or Nimo-CAR⁺ T cells. Significant extension in survival in Nimo-CAR⁺ T cell treatment group determined by Mantel-Cox log-rank test, p=0.0269.

FIGS. 29A-C. Engraftment of U87 and CAR⁺ T-cell phenotype prior to T-cell treatment. (A) Four days after tumor injection, tumors were imaged by BLI following injection with D-luciferin and 10 minute incubation. (B) Mice were divided into three groups to evenly distribute relative tumor burden as determined by day 4 BLI flux measurements. (C) Cetux-CAR⁺ and Nimo-CAR⁺ T cells expanded through 4 stimulation cycles were evaluated for CAR expression and CD4/CD8 ratio by flow cytometry.

FIGS. 30A-B. Cetux-CAR⁺, but not Nimo-CAR⁺ T cells inhibit growth of U87 intracranial xenografts(A) Serial BLI assessed relative size of tumor. (B) Relative tumor growth as assessed by serial BLI of tumor. Significant difference in BLI between mice with no treatment vs. treatment (n=6) with Cetux-CAR⁺ T cells (n=6, p<0.01) reached at day 25, two-way ANOVA (Sidak’s post-test).

FIG. 31. Survival of mice bearing U87 intracranial xenografts treated with Cetux-CAR⁺ and Nimo-CAR⁺ T cells. Survival of mice with U87-ffLuc-mKate intracranial xenografts receiving no treatment, Cetux-CAR⁺ T cells or Nimo-CAR⁺ T cells. Significant extension in survival in Cetux-CAR⁺ T cell treatment group determined by Mantel-Cox log-rank test, p=0.0150.

FIG. 32. Summary of strategies to safely expand repertoire of antigens for CAR⁺ T cell therapy. Strategies fall into three main categories: (i) limiting CAR expression by drug-induced suicide or transient CAR expression, (ii) targeting CAR to tumor site by limiting expression to hypoxic regions or co-expressing homing receptors, and (iii) limiting CAR activation by splitting signals to require two antigens to recognize tumor, expressing an inhibitory CAR to prevent activation to normal tissue, or expressing CAR conditionally activated by high antigen density.

FIGS. 33A-F. Vector maps of constructed plasmids. (A) Cetuximab-derived CAR transposon. Annotated as follows: HEF-1α/p: promoter for human elongation factor-1α; BGH: bovine growth hormone poly adenylation sequence; IR/DR: inverted repeat/direct repeat; ColE1: a minimal E.coli origin of replication; Kan/R: gene for kanamycin resistance; Kan/p: promoter for kanamycin resistance gene. (B) Nimotuzumab-derived CAR transposon. Annotated as follows: HEF-1a/p: promoter for human elongation factor-1α; BGH: bovine growth hormone poly adenylation sequence; IR/DR: inverted repeat/direct repeat; ColE1: a minimal E.coli origin of replication; Kan/R: gene for kanamycin resistance; Kan/p: promoter for kanamycin resistance gene.(C) Cetuximab-derived CAR/pGEM-A64 plasmid. Annotated as follows: amp/R: gene for ampicillin resistance, SpeI: restriction site for linearization. (D) Nimotuzumab-derived CAR/pGEM-A64 plasmid. Annotated as follows: amp/R: gene for ampicillin resistance, SpeI: restriction site for linearization. (E) tEGFR-F2A-Neo transposon. Annotated as follows: HEF-1α/p: promoter for human elongation factor-1α; BGH: bovine growth hormone poly adenylation sequence; F2A: self-cleavable peptide F2A; Neo/r: gene for neomycin resistance; IR/DR: inverted repeat/direct repeat; ColE1: a minimal E.coli origin of replication; Kan/R: gene for kanamycin resistance; Kan/p: promoter for kanamycin resistance gene. (F) CAR-L transposon. Annotated as follows: HEF-1α/p: promoter for human elongation factor-1α; Zeocin R: gene for zeomycin resistance; BGH: bovine growth hormone poly adenylation sequence; IR/DR: inverted repeat/direct repeat; ColE1: a minimal E.coli origin of replication; Kan/R: gene for kanamycin resistance; Kan/p: promoter for kanamycin resistance gene.

FIG. 34. Vector map of pLVU3G-effLuc-T2A-mKateS158A. Annotations are as follows: B1: Gateway donor site B1; effLuc: enhanced firefly luciferase; T2A: T2A ribosomal slip site; mKateS158A: enhanced mKate red fluorescent protein; B2: Gateway donor site B2, HBV PRE: Hepatitis B post-translational regulatory element; HIV SIN LTR: HIV self-inactivating long terminal repeat; ampR: ampicillin resistance; LTR: long terminal repeat; HIV cPPT: HIV central polypurine tract.

FIG. 35. Standard curve for relating MFI to ABC for quantitative flow cytometry. Following incubation with saturating amounts anti-EGFR-PE, microsphere bead standard samples with known antibody binding capacity were acquired on flow cytometer. Standard curve was generated by plotting known antibody binding capacity against measured mean fluorescence intensity acquired by flow cytometry.

### DETAILED DESCRIPTION

### I. Aspects of the Embodiments

### A. Transient Expression of EGFR-specific CAR by RNA-modification

Transient expression of CAR by RNA transfer has been proposed to reduce the potential for long-term, on-target, off-tissue toxicity of CAR T cell therapy directed against antigens with normal tissue expression. Numeric expansion of T cells prior to RNA transfer is appealing to obtain clinically relevant T cell numbers needed for patient infusion. The inventors explored numeric expansion of T cells independent of antigen-specificity by co-culturing on aAPC loaded with anti-CD3 antibody, OKT3. Altering the ratio of antigen presenting cells (e.g., aAPCs) to T cells in culture altered the phenotype of the resultant T cell population. T cells expanded with low density of aAPC (10 T cells to 1 aAPC) were associated with increased proportion of CD8⁺ T cells, increased presence of central memory phenotype T cells, reduced production of IFN-γ and TNF-α, but increased production of IL-2, and potentially less clonal loss of TCR diversity following expansion relative to T cells expanded with high density aAPC. T cells expanded with low density aAPC were more amenable to RNA electro-transfer, demonstrating higher expression of RNA transcripts and improved T-cell viability following electro-transfer than T cells expanded with high density aAPC.

A potential benefit of use of aAPC for T-cell expansion is the ability to form stable interactions with T cells by virtue of expression of adhesion molecules LFA-3 and ICAM-1 (Suhoski et al., 2007; Paulos et al., 2008). Additionally, aAPC can be modified with relative ease to express desired arrays of costimulatory molecules. Thus, aAPC for numeric T-cell expansion provides a platform to evaluate various combinations of costimulatory molecules for T-cell expansion to achieve an optimal T-cell phenotype for adoptive T-cell therapy. In addition to modification of aAPC, the inventors have described the impact of the density of aAPC in T cell culture on the phenotype of resulting T-cell populations. While CD8⁺ T cells, or cytotoxic T cells, are often thought of as the ideal T-cell population for anti-tumor immunotherapy, evidence suggests that CD8⁺ T cells require CD4⁺ T-cell help in vivo to achieve optimal anti-tumor response and memory formation (Kamphorts et al., 2013; Bourgeois et al., 2002; Sun et al., 20013). However, the ideal ratio of CD4⁺ to CD8⁺ T cells is unknown (Muranski et al., 2009). By altering density of aAPC in expansion cultures to skew CD4/CD8 ratio in T cells for adoptive immunotherapy, whether they be TIL isolated from patients or gene-modified T cells, these questions may be addressed in clinical trials. Finally, reducing density of aAPC in culture resulted in more T cells with a central memory phenotype (CCR7⁺CD45RA^{neg}) than T cells expanded with higher density of aAPC. While the benefit of enhanced persistence of central memory phenotype T cells may not extend to RNA-modified T cells, which are only transiently redirected for tumor antigen, persistence of T cells has been shown to improve the anti-tumor efficacy of T-cell therapy (Kowolik et al., 2006; Robbins et al., 2004; Stephan et al., 2007; Wu et al., 2013). Therefore, ex vivo expansion with low density aAPC may be used to reprogram stably genetically modified T cells or TIL to a central memory phenotype for enhanced persistence.

Expression of CAR by RNA-modification in ex vivo expanded T cells was found to be more variable than expression of CAR by non-viral DNA-modification and expansion of T cells through CAR recognition of antigen. Expression of CAR at different densities did not impact the ability of the T cells to specifically lyse targets, although it is reasonable to expect that below a certain threshold, low CAR expression would have a negative impact on specific lysis of targets, as previously reported (Weijtens et al., 2000). Others have described tunable expression of CAR by RNA modification of T cells, such that the dose of RNA determines the level of transgene expression (Rabinovich et al., 2006; Yoon et al., 2009; Barrett et al., 2011). RNA modification of T cells in the present study was conducted using the same quantity of RNA, therefore, this does not account for variability of CAR expression by altering RNA dose. Instead, it is likely that variability between donors accounts for differences in CAR expression intensity following electro-transfer. The presently described protocol for T-cell expansion prior to RNA transfer may play a role in altering the sensitivity of T cells from certain donors to RNA uptake, and increasing the RNA quantity in electro-transfers may increase expression of CAR in these donors. High expression of CAR by transferring relatively high quantities of RNA can result in prolonged CAR expression and CAR-mediated activity over a prolonged period of time (Barrett et al., 2011). Prolonged CAR expression from RNA transfer may be beneficial to anti-tumor activity, particularly since stimulation of T cells seems to accelerate the loss of CAR expression. However, prolonging the expression of CAR may also increase T-cell activity in response to normal tissue antigen requiring the optimization of CAR expression to determine the optimal duration of expression to maximize anti-tumor activity while reducing normal tissue toxicity.

RNA-modification of T cells did not alter the proportion of effector memory and central memory T cells found in ex vivo expanded T cells prior to electro-transfer of RNA, similar to previous reports (Schaft et al., 2006). Only T cells expanded at relatively low aAPC density, 10 T cells to 1 aAPC, were capable of efficient RNA transcript uptake without significant toxicity, even with various electroporation conditions. This population of T cells also demonstrated a substantial proportion of T cells with a central memory phenotype (CCR7⁺CD45RA^{neg}) that had reduced production of IFN-γ and TNF-α, and cytotoxic effector molecules granzyme B and perforin. As a result, RNA-modified T cells contained significantly more central memory phenotype T cells than DNA-modified T cells, demonstrated reduced production of IFN-γ and TNF-α in response to EGFR-expressing cells and slightly less specific lysis at low E:T ratios. Thus, the precursor T cell population for RNA-modification has a strong influence on CAR-mediated T cell function following RNA transfer and the reduced cytokine production and slightly less specific lysis of RNA-modified T cells may translate to reduced anti-tumor efficacy in an in vivo model where cytotoxic potential of T cells is short-lived and the enhanced persistence of a central memory T cell population may not be beneficial. RNA-modification of T cells expanded at 1 T cell to 2 aAPC, which demonstrated a more significant proportion of effector memory phenotype T cells, similar to DNA-modified CAR⁺ T cells, and consequently the capacity for higher production of IFN-γ and TNF-α is desirable. The addition of cytokines prior to RNA transfer may improve viability and additional electroporation programs may efficiently transfer RNA into these T cells.

Cetux-CAR introduced to T cells through RNA transfer was transiently expressed, and loss of expression was accelerated by stimulus to T cells, including addition of cytokines IL-2 and IL-21 and antigenic-stimulus through addition of EGFR-expressing cell lines. Concomitant with loss of CAR expression, RNA-modified T cells demonstrated reduced cytotoxicity against EGFR-expressing cell lines, including tumor cells and normal human renal cells. One concern for the use of RNA-modified T cells is that their inherently reduced capacity to target tumor over time will result in reduced anti-tumor efficacy relative to stably-modified T cells. Multiple injections of T cells modified to express a mesothelin-specific CAR by RNA transfer for the treatment of a murine model of mesothelioma demonstrated that biweekly, intratumoral injections demonstrated control of tumor growth, but following cessation of treatment, tumors relapsed (Zhao et al., 2010). Treatment of an in vivo disseminated leukemia murine model has demonstrated that while RNA-modified CAR⁺ T cells specific for CD19 have anti-tumor activity after a single injection, tumors often relapse after a time period consistent with CAR degradation (Barrett et al., 2011). In contrast, a single intratumoral injection of T cells stably expressing mesothelin-specific CAR mediated superior anti-tumor activity and was capable of curing most mice. Optimization of dosing of RNA-modified T cells demonstrated that a combination of cyclophosphamide to eliminate residual CAR^{neg} T cells before subsequent infusions and a weighted, split-dosing regimen was more effective in controlling disease burden, and was similar in anti-tumor efficacy to stably modified T cells (Barrett et al., 2013). Thus, optimizing a dosing regimen can improve the anti-tumor activity of RNA-modified T cells.

### B. CAR⁺ T Cells Can Distinguish Malignant Cells From Normal Cells Based on EGFR Density

Cetux-CAR⁺ T cells can recognize normal tissue antigen, which could result in on-target, off-tissue toxicity. Thus, the inventors investigated expression of CAR as RNA species as a method to control on-target, off-tissue toxicity through transient expression of CAR. While CAR expression was transient and reduced potential for cytotoxicity against normal tissue EGFR after degradation of CAR, it did not address the potential for immediate T-cell effector function upon recognition of normal tissue EGFR before considerable degradation of CAR. Additionally, by limiting CAR expression, T cells are rendered non-responsive to EGFR-expressing tumor following CAR degradation, and the potential for lasting anti-tumor activity is compromised by this approach. Therefore, mechanisms to control CAR activity in the presence of normal tissue to limit deleterious on-target, off-tissue toxicity without compromising anti-tumor activity were investigated.

Endogenous T cell activation is dependent on both affinity of the TCR and density of peptide presented via MHC (Hemmer et al., 1998; Viola et al., 1996; Gottschalk et al., 2012; Gottschalk 2010). T cells are activated by a cumulative signal through the TCR that surpasses a certain threshold required for elicitation of effector functions Hemmer et al., 1998; Rosette et al., 2001; Viola et al., 1996). For high affinity TCRs, relatively low antigen density is sufficient to trigger T cell responses; however, low affinity TCRs required higher antigen density to achieve similar effector T cell responses (Gottschalk et al., 2012). Many tumors overexpress TAA at higher densities than their normal tissue expression (Barker et al., 2001; Lacunza et al., 2010; Hirsch et al., 2009). Amplification and overexpression of EGFR in glioma highlight this relationship as EGFR is overexpressed in glioma relative to normal tissue, and overexpression correlates with tumor grade, such that grade IV glioblastoma expresses the highest density of EGFR (Smith et al., 2001; Hu et al., 2013; Galanis et al., 1998). Therefore, the inventors determined if EGFR-specific CAR-modified T cells could distinguish malignant cells from normal cells based on EGFR density by reducing the binding affinity of the CAR.

The portion of Cetux-CAR that endows antigenic specificity is derived from the scFv portion of the monoclonal antibody cetuximab, which is characterized by a high affinity (Kd=1.9x10⁻⁹) (Talavera et al., 2009). Therefore, the inventors generated a CAR from the monoclonal antibody nimotuzumab, which shares a highly overlapping epitope with cetuximab and a 10-fold lower dissociation constant (Kd=2.1x10⁻⁸), characterized by a 59-fold reduced rate of association (Talavera et al., 2009; Garrido et al., 2011; Adams et al., Zuckier et al., 2000). The reduced association rate and subsequent reduction in overall affinity imposes a requirement for bivalent recognition of EGFR, which only occurs when EGFR is expressed at high density. Thus, a CAR derived from nimotuzumab may enable T cells to distinguish malignant tissue from normal tissue based on density of EGFR expression.

Recent clinical success in CLL and ALL note persistent B-cell aplasia in patients with complete tumor response to CD19-CAR⁺ T-cell therapy, but this toxicity is considered tolerable as CD19 is a lineage-restricted antigen and B cell aplasia is considered a tolerable toxicity in the setting of advanced lymphoma (Grupp et al., 2013; Porter et al., 2011). Serious adverse events in clinical trials targeting HER2 and CAIX with CAR-modified T cells highlights the need to control CAR T-cell activity against normal tissue antigen expression in order to broaden the range of safely targetable antigens beyond lineage and tumor restricted antigens (Lamers et al., 2013; Morgan et al., 2010). Aberrantly expressed TAAs are often overexpressed on tumor relative to normal tissue, such as EGFR expression in glioblastoma (Smith et al., 2001; Hu et al., 2013; Galanis et al., 1998). The inventors developed a CAR specific to EGFR with reduced capacity to respond to low antigen density to minimize the potential for normal tissue, while maintaining adequate effector function in response to high antigen density. This was accomplished by developing an EGFR-specific CAR from nimotuzumab, a monoclonal antibody with a highly-overlapping epitope, yet reduced binding kinetics compared to cetuximab (Talavera et al., 2009; Garrido et al., 2011). While Cetux-CAR⁺ T cells were capable of targeting low and high EGFR density, Nimo-CAR⁺ T cells were able to tune T-cell activity to antigen density and response was dependent on EGFR density expressed on target cells. While Nimo-CAR⁺ T cells demonstrate reduced activity relative to Cetux-CAR⁺ T cells in response to low EGFR density on tumor cells and normal renal cells, they were capable of equivalent redirected specificity and function in response to high EGFR density. CAR affinity influenced proliferation after antigen challenge and Cetux-CAR⁺ T cells demonstrated impaired proliferation when compared with Nimo-CAR⁺ T cells after antigen challenge, but not increased propensity for activation induced cell death (AICD). Additionally, CAR affinity influences downregulation of CAR from T-cell surface after interaction with antigen. Cetux-CAR exhibited more rapid and prolonged downregulation from the cell surface after interaction with high EGFR density than Nimo-CAR. Cetux-CAR⁺ T cells had impaired ability to respond to re-challenge with antigen, which could be a result of downregulated CAR or potentially functional exhaustion of Cetux-CAR⁺ T cells (James et al., 2010; Lim et al., 2002).

Complications in delineating the impact of scFv on CAR function stem from considerable debate surrounding the biochemical parameter of endogenous TCR binding that best predicts T-cell function. The kinetics of TCR binding can be described by the equation:

$${K}_{d} = \frac{{k}_{o f f}}{{k}_{o n}}$$

such that the dissociation constant, Kd, is equal to the ratio of the rate of dissociation (koff) and the rate of association (kon) (14). Both the dissociation constant (Kd) and the dissociation rate (koff) have been reported as important determinants of T-cell function following TCR recognition of pepMHC, however these two parameters are often strongly correlated, so it is difficult to separate their respective impact on T-cell function (Kersh et al., 1998; McKeithan T.W. 1995; Nauerth et al., 2013). The kinetic proofreading model of T-cell triggering states that koff impact T-cell function, such that sufficiently long dwell time is required to trigger T-cell signaling and activation. This has been amended to include a window of optimal dwell time, in which prolonged dwell time may be detrimental to T-cell activation by impairing the ability of serial triggering of multiple TCR by a single pepMHC complex (Kalergis et al., 2001). However, these models are contradicted by reports of very short dwell time interactions capable of producing functional T-cell responses (Govern et al., 2010; Tian et al., 2007; Aleksic et al., 2010; Gottschalk et al., 2012). Recent analysis aiming to reduce previous dataset bias by reducing the high degree of correlation between Kd and koff values and expanding dynamic range of kon values uncovered an important role in contribution of kon to T-cell activation, encompassed in a T-cell confinement model of T-cell triggering, in which T-cell function is directly correlated with the duration of T-cell confinement derived from a mathematical relationship between rate of association, rate of dissociation, and diffusion of TCR and pepMHC in their relative membranes (Tain et al., 2007; Aleksic et al., 2010). Interestingly, as kon becomes low, TCR and pepMHC are able to diffuse in their relative membranes before rebinding, thus the duration of interaction reduces to the koff value. In contrast, as kon becomes high, the TCR is capable of rapid rebinding to extend the dwell time, and the duration of interaction and resulting T-cell function is best predicted by Kd. This ongoing debate to define the role of TCR affinity components that control T-cell functional avidity cautions against universal models relying on one biochemical parameter of binding as a superior indicator of function over others. Instead, it is likely a combination of rates of association and dissociation as well as density of antigen freely moving through target cell membrane that defines functional response.

Endogenous TCR responses are generally described as much lower affinity than the binding of monoclonal antibodies, which are used to derive CAR specificity (Stone et al., 2009). However, SPR techniques used to measure TCR binding affinity are typically performed in three dimensions, and do not recapitulate the physiological interaction of a T cell with an antigen presenting cell, in which both binding partners are constrained in their respective membranes, increasing the probability of binding due to constrained intercellular space and proper molecule orientation (Huppa et al., 2010). Measurement of TCR binding kinetics in 2D suggests that TCR binding is of higher affinity than suggested by 3D measurements characterized by increased rates of association and decreased rates of dissociation (Huang et al., 2010; Robert et al., 2012). However, binding kinetics of other ligand/receptor pairs, such as ICAM-1 or LFA-1 did not show a difference between affinity measurements taken in 3D or 2D assays. Interestingly, ablation of cytoskeletal polymerization reduces measurements made in 2D to measurements made in 3D, highlighting the role of dynamic cellular and cytoskeletal processes in enhancing T cell binding to antigen (Robert et al., 2012). Whether similar cytoskeletal interactions or enhancement of binding affinity of CAR occurs is currently unknown, and therefore, it is unclear if assumptions made about binding affinity of the scFv domain of CAR can be directly made from measurements of monoclonal antibody affinity in 3D assays. In addition, several factors contribute to enhance overall T cell binding avidity, such as co-receptor binding to MHC and TCR nanocluster and microcluster formation on the T-cell surface prior to and following T cell activation (Holler et al., 2003; Schamel et al., 2005; Schamel et al., 2013; Kumar et al., 2011; Yokosuka et al., 2010). While it appears that CARs can be expressed in oligomeric form on the T cell surface, the degree of involvement of CAR with endogenous T cell signaling complexes is unclear. While reports of first generation CARs, signaling through only CD3-ζ demonstrate a requirement for association with endogenous CD3-ζ to achieve CAR-dependent T-cell activation, second generation CARs signaling through transmembrane CD28 and intracellular CD28 and CD3-ζ demonstrate no difference in CAR-dependent activation ability when endogenous TCR-CD3 complexes are restricted from the T cell surface (Bridgeman et al., 2010; Torikai et al., 2012). Therefore, the association of CAR with endogenous TCR signaling machinery may be dependent on CAR configuration.

Specific studies addressing the role of scFv affinity in CAR design are limited, and focus on contribution of the dissociation constant, Kd. Recent studies with ROR1-specific CAR compared a with 6-fold lower Kd, thus higher affinity, resulting from both increased kon and decreased koff and demonstrated that higher affinity ROR-1specific CAR increased T-cell function in vitro, including production of cytokines and specific lysis, without increased propensity for AICD (Hudecek et al., 2013). Additionally, high affinity ROR-1-specific CAR⁺ T cells mediated superior anti-tumor activity in vivo. Similarly, the higher affinity of Cetux-CAR⁺ T cells did not increase propensity for AICD, and had increased T-cell function, including production of cytokines and specific lysis, in response to reduced EGFR density. However, a previous study of a series of CARs derived from a panel of affinity-matured HER2-specific monoclonal antibodies with a wide range of Kd values, found that an affinity threshold existed, below which CAR-dependent T-cell activation was impaired; however, above this threshold, activation of T cells in response to various levels of HER2 did not improve with increased affinity (Chmielewski et al., 2004). In contrast, the present study identified different ability of high affinity CAR and low affinity CAR to target based on antigen density. Higher affinity Cetux-CAR⁺ T cells were associated with increased cytokine production and specific lysis in response to reduced EGFR density relative to Nimo-CAR⁺ T cells. While, Nimo-CAR is lower affinity relative to Cetux-CAR, the Kd value of Nimo-CAR was above the affinity threshold and within the range predicted to have effector function by the previous study. Similar to studies with endogenous TCRs, these results indicate that descriptions of CAR affinity should not be described solely by the dissociation constant, and support that relationship between individual dissociation and association rates be taken into consideration for CAR design.

The contradictions between the influence of affinity on CAR function between studies may be explained by the distinct relationships of the biochemical parameters koff and kon that constitute the dissociation constant Kd. The HER2-specific CARs were derived from antibodies that displayed a wide range of Kd values differing primarily in koff, with minimal correlation of kon values (Chmielewski et al., 2004). Thus, higher affinity interactions did not have increased rates of association, but increased duration of interaction with antigen. In contrast, the higher affinity of the ROR-1-specific CAR and Cetux-CAR were both influenced by increased association rates of binding. The higher affinity monoclonal antibody used to derive the ROR-1-specific CAR had a 6-fold lower Kd, from contributions of both increased kon and decreased koff, such that the higher affinity was characterized by both increased association rates and increased duration of interaction (Hudecek et al., 2013). The 10-fold difference in Kd between cetuximab and nimotuzumab is primarily impacted by a 59-fold increase in the kon and a 5.3x increase in the koff of cetuximab, such that cetuximab has greatly enhanced rate of association relative to nimotuzumab, but in contrast to most higher affinity interactions, a shorter duration of interaction (Talavera et al., 2009). Therefore, altering association rate rather than the dissociation rate of scFv domain in CAR design may have a greater impact on T-cell function.

Previous studies have established that a minimum CAR density is required for T-cell activation, below which T-cell activation is abrogated (James et al., 2010). However, sufficiently high antigen expression can mitigate this requirement and achieve CAR-dependent T-cell activation when CAR is expressed at low density (James et al., 2010). The interplay between CAR expression density, antigen density and CAR affinity and impact on CAR⁺ T cell function were evaluated in a study using high and low affinity HER-specific CARs. This study reported that reduced T-cell function of T cells with low CAR density in response to low antigen density was only apparent when T cells expressed a higher affinity HER2-specific CAR (Turatti et al., 2007). However, when CAR was expressed at higher density, CAR-mediated cytotoxicity was irrespective of affinity or antigen density. The authors attributed the reduced response of high affinity CAR when expressed low density to low HER2 density to a failure to induce serial triggering. Although it has been reported that CARs to do not serially trigger as endogenous TCRs (James et al., 2010), it is possible that this is CAR-specific, and that different transmembrane regions, endodomains, and scFv affinity may impact ability to serially trigger. The inventors did not observe any defect in Cetux-CAR⁺ T cells in initial response to low antigen density, however, the level of CAR expression culled out through repetitive stimulation on EGFR⁺ aAPC may select for an optimum CAR density, with T cells expressing suboptimal levels of CAR failing to expand and thus falling out of the repertoire. In contrast, the present findings suggest that the lower affinity Nimo-CAR⁺ T cells demonstrate reduced sensitivity to low antigen expression, but increasing density of Nimo-CAR did not restore Nimo-CAR⁺ T cell sensitivity to low antigen, thus it is likely controlled by a different mechanism.

Although expression of CAR at low density can reduce sensitivity to antigen, this is not likely to be an optimal strategy selectively target high antigen density in vivo, primarily because CAR expressed at low density demonstrate reduced sensitivity to all levels of antigen, and therefore the potential for reduced anti-tumor activity (James et al., 2010; Weijtens et al., 2000). Additionally, CAR downregulates from the T-cell surface at a constant number of CAR/antigen (James et al., 2010). Thus, T cells expressing CAR at lower density are more susceptible to downregulation below the minimum density to achieve T-cell activation.

In this study, Nimo-CAR, predicted to have lower affinity due to reduced association rate of binding relative to Cetux-CAR, mediated T-cell activation that directly correlated with EGFR expression density and reduced activity in response to normal renal cells with low EGFR density. Additionally, Nimo-CAR⁺ T cells showed enhanced proliferation and reduced CAR downregulation relative to Cetux-CAR⁺ T cells. Targeting EGFR on glioblastoma by Nimo-CAR⁺ T cells has the potential to mediate anti-tumor activity while reducing the potential for on-target, off-tissue toxicity.

### C. In Vivo Anti-tumor Efficacy of Cetux-CAR⁺ and Nimo-CAR⁺ T cells in an Intracranial Glioma Model

Some tumors, such as glioblastoma, overexpress EGFR at a higher density relative to normal tissue expression and hypothesized that altering scFv domain of CAR to reduce binding affinity could preferentially activate T cells in the presence of high EGFR density but reduce T cell activity in the presence of low EGFR density. Cetux-CAR and Nimo-CAR bind overlapping epitopes on EGFR with distinct affinities and binding kinetics, such that Cetux-CAR has a 5.3-fold lower dissociation constant, and therefore higher affinity, characterized by a 59-fold higher rate of association. In vitro studies also demonstrated Cetux-CAR had reduced proliferation in response to antigen in the absence of exogenous cytokine, enhanced downregulation of CAR that was dependent on scFv domain of CAR binding EGFR and density of EGFR, and impaired cytokine production in response to re-challenge with antigen.

Evaluation of efficacy of Cetux-CAR⁺ and Nimo-CAR⁺ T cells in treatment of intracranial glioma xenografts supported in vitro conclusions by demonstrating that both Cetux-CAR⁺ T cells and Nimo-CAR⁺ T cells can mediate anti-tumor activity against U87med, expressing intermediate EGFR density, but only Cetux-CAR⁺ T cells demonstrated anti-tumor activity against U87 with endogenously low EGFR density.

Some studies have demonstrated that higher affinity TCR interactions can result in superior in vivo activity (Nauerth et al., 2013; Zhong et al., 2013); however, it has been demonstrated that in vitro T-cell activity does not always mirror in vivo efficacy (Chervin et al., 2013; Janicki et al., 2008). High affinity T cells with high potency in vitro have been shown to have attenuated responses in vivo, characterized by decreased signaling, expansion and T-cell mediated function (Corse et al., 2010). Similarly, low affinity interaction have been demonstrated to have curtailed T-cell expansion in vivo, resulting in fewer T cells present at each stage of the immune response (Zehn et al., 2009). Models assessing the role of TCR affinity in anti-tumor efficacy have demonstrated that high affinity TCR interactions have impaired anti-tumor function, characterized by decreased presence in tumor and impaired cytolytic function (Chervin et al., 2013; Engels et al., 2012; Janicki et al., 2008). Thus, it has been suggested that T cells with intermediate affinity may better control tumor growth relative to high affinity T cells (Corse et al., 2010; Janicki et al., 2008). Combining these observation with in vitro observations that Cetux-CAR⁺ T cells have decreased proliferative capacity when stimulated in the absence of exogenous cytokine, enhanced CAR downregulation following engagement with antigen, and reduced ability to respond to re-challenge with antigen, Cetux-CAR⁺ T cells may have reduced anti-tumor efficacy in vivo. The inventors did not observe impaired anti-tumor efficacy relative to Nimo-CAR⁺ T cells; however, the fate of CAR⁺ after intratumoral injection was not followed, and therefore, differences in vivo expansion were not assessed. Intratumoral injection of CAR⁺ T cells was chosen to avoid the confounding variable of disparate abilities of CAR⁺ T cells to home to tumor when evaluating anti-tumor activity; however, it is possible that Cetux-CAR⁺ T cells may have reduced tumor infiltration due to retention in tumor periphery.

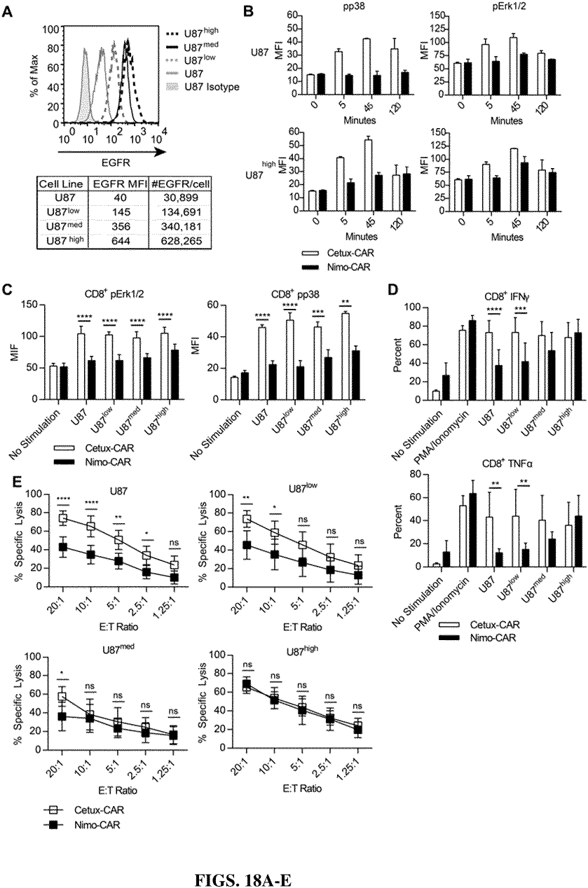

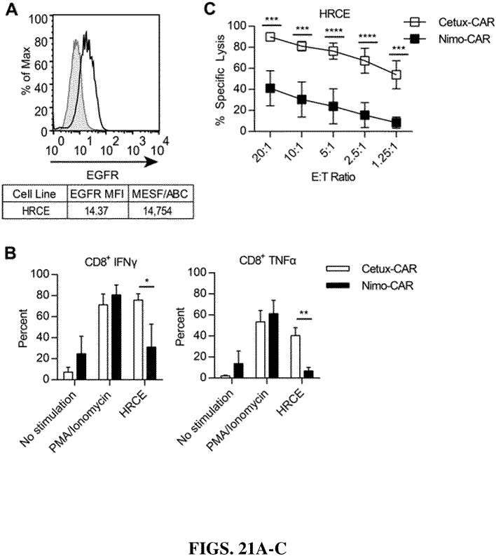

Nimo-CAR⁺ T cell treatment did not significantly reduce tumor burden or improve the survival of mice relative to untreated mice in response to low EGFR density on U87, which is about 2-fold higher than EGFR density measured on normal renal epithelial cells (FIG. 18 and FIG. 21). In contrast, Cetux-CAR⁺ T cells demonstrated tumor control and extended survival in 3/6 mice with low EGFR density. While Nimo-CAR⁺ T cell treatment may have reduced cytotoxic potential against normal tissue with very low EGFR density, they also have the potential for tumor escape variants expressing low EGFR density. However, due to the substantial heterogeneity in glioblastoma, it is unlikely for a single target to be expressed on all of the tumor cells within a given patient (Little et al., 2012; Szerlip et al., 2012). Treatment of experimental glioblastoma models with HER2-specific CAR⁺ T cells has also demonstrated escape of HER2null tumor cells (Ahmed et al., 2010; Hegde et al., 2013). Profiling patient tumors can identify combinations of antigens to target the maximum number of cells in a given tumor, and targeting multiple antigens by CAR⁺ T cells has been shown to improve treatment efficacy of treatment of CAR⁺ T cells with single specificity (Hegde et al., 2013). In vivo experimentation with U87 with uniform EGFR density does not recapitulate antigen heterogeneity in patient tumors, therefore, evaluation of Cetux-CAR⁺ T cells or Nimo-CAR⁺ T cells in combination with CAR⁺ T cells of different specificities can be evaluated against glioblastoma specimens derived from patients that may better recapitulate tumor heterogeneity in vivo (Ahmed et al., 2010).

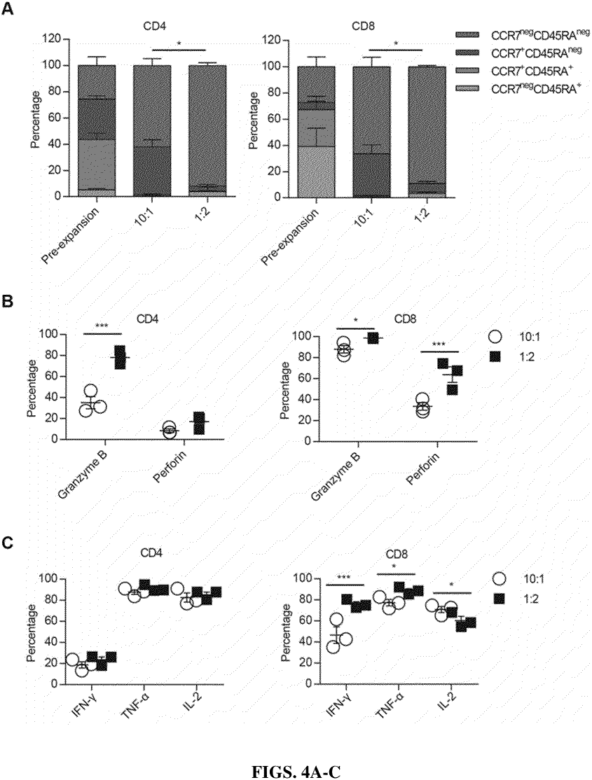

Unexpectedly, Cetux-CAR⁺ T cells showed significant toxicity within 7 days of T cell treatment, with 6/14 mice dying within 7 days of a T-cell injection. Previously, an EGFR-specific CAR has been reported to have no detectable in vivo toxicity by measurement of liver enzymes 48 hours after T-cell infusion in mice bearing no tumor (Zhou et al., 2013). Because this CAR was derived from a murine antibody, it is unlikely that the EGFR-specific CAR would recognize murine EGFR on normal tissue. Additionally, measurement of toxicity in the absence of antigen does not replicate physiologic CAR⁺ T-cell activation in patients expressing antigen on tumors, as these cells will activate, proliferate, and produce cytokine in response to tumor lysis, which could all contribute to measureable toxicity (Barrett et al., 2014). In fact, in the present study, treatment of mice with Cetux-CAR⁺ T cells bearing low antigen tumor or no tumor did not result in detectable toxicity (FIG. 4), highlighting the role of in vivo T-cell activation to observed T-cell toxicity.

Because cetuximab does not recognize murine EGFR, on-target, off-tissue toxicity is not likely a cause of Cetux-CAR⁺ T cell-related toxicity (Mutsaers et al., 2009). Possible mechanisms for Cetux-CAR mediated toxicity in this model include cytokine-related toxicity resulting from T cell activation or possibly enhanced avidity of Cetux-CAR due to clustering, immune synapse formation or association with T-cell cytoskeleton that reduces antigenic-specificity, as has been described in the contribution of CD8 coreceptor binding to enhance avidity of high affinity TCRs, resulting in loss of specificity (Stone et al., 2013).

In summary, Nimo-CAR⁺ T cells demonstrate anti-tumor activity and improved survival comparable to higher affinity Cetux-CAR⁺ T cells in an intracranial orthotopic xenograft model, without T-cell related toxicity associated with Cetux-CAR⁺ T cells. In contrast, Cetux-CAR⁺ T cells, but not Nimo-CAR⁺ T cells demonstrate anti-tumor activity against tumors with low EGFR density. These findings are consistent with in vitro observations that Nimo-CAR⁺ T cells have reduced activity in response to low EGFR density.

### D. Safely Expanding the Repertoire of Antigens for CAR⁺ T-cell Therapy

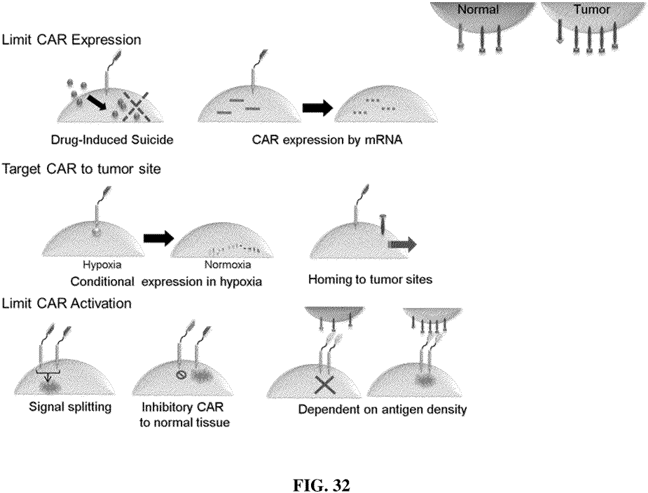

Methods developed to achieve safety of CAR⁺ T cells can be categorized into three main strategies: (i) restricting CAR⁺ T cells to tumor tissue, (ii) limiting CAR expression/T cell persistence, and (iii) restricting CAR-mediated T cell activation to tumor (FIG. 32). Co-expression of homing molecules with CAR in T cells to home to site of the tumor, such as CCR2, CCR4 and CXCR2, has been described to sequester CAR⁺ T cells to site of the tumor (Peng et al., 2010; Moon et al., 2011; Di Stasi et al., 2009). While CAR⁺ T cells are enriched in tumor tissue when compared with CAR⁺ T cells without homing receptors, it is unclear what percentage of CAR⁺ T cells expressing homing receptors do not efficiently home to the tumor and could, therefore, target normal tissue. Likewise, chemokines secreted by tumors can also be secreted in normal tissue during tissue trauma and healing. Therefore, combining these treatments with other treatment modalities, such as surgery, chemotherapy and radiation would risk attracting T cells to normal tissue non-specifically injured during treatment. Development of CAR preferentially expressed in hypoxic condition, common in many tumors, has been achieved by fusing CAR to an oxygen-dependent degradation domain to limit CAR expression and capacity to target tissue in normoxia (Chan et al., 2005). Because CAR degradation in T cells moving from hypoxia to normoxia may take minutes to hours, it is feasible for on-target, off-tissue toxicity may occur prior to CAR degradation. In addition, while the center of many tumors are hypoxic, well-vascularized peripheral tumor regions may have sufficient oxygen concentration to degrade CAR, protecting peripheral regions from CAR-mediated T-cell activity (Vartanian et al., 2014).

Strategies to temporally limit CAR⁺ T cell presence include suicide gene modification of T cells, such as expression of CAR as a transient RNA species, and introduction of iCaspase9 suicide switch, which is specifically activated by a chemical inducer of dimerization (CID) to result in T-cell death (Zhao et al., 2010; DiStassi et al., 2011; Budde et al., 2013; Barrett et al., 2011; Barrett et al., 2013). Both methods have high penetrance and result in almost complete abrogation of CAR⁺ T cells, either after induction of apoptosis by drug delivery or loss of RNA transgene expression over time. Because both strategies permanently ablate CAR⁺ T cells, they also limit therapeutic efficacy against tumor while protecting normal tissue. One limitation of these strategies is that before CAR reduction or T cell ablation, potent activity against normal cells exists, and there is no short-term limitation of toxicity. Serious adverse events from T-cell therapy can progress rapidly from onset of clinical symptoms, therefore, it is desirable to have a strategy to protect normal tissue from the moment of CAR⁺ T-cell infusion (Grupp et al., 2013; Porter et al., 2011).

Dual-specific, complementary CARs have achieved selective activation in response to co-expression of two antigens mutually expressed only on tumor by dissociating signaling domains and expressing two chimeric receptors with two specificities. In this strategy, one specificity is fused to CD3ζ to express a first generation CAR and a different, complementary specificity is fused to costimulation endodomains, termed a chimeric costimulation receptor (CCR), such that full activation and T-cell function is only attained with simultaneous engagement of CAR and CCR by co-expression of by antigens (Wilkie et al., 2014; Lanitis et al., 2013; Kloss et al., 2013). This approach has been piloted with different pairs of CAR and CCR with redirected specificities towards HER2 and MUC1 for breast cancer, PSMA and PSCA for prostate cancer and mesothelin and α-folate receptor for ovarian cancer treatment. Early studies have demonstrated that T-cell activation and lytic function can occur against single antigen expressing targets via first generation CAR expression in the absence of CCR activation. Although this cytotoxicity is lower than that observed with second generation CARs, there is still some residual risk of CAR targeting normal tissue expressing single antigen (Wilkie et al., 2014; Lanitis et al., 2013). One strategy to overcome this limitation is to develop a first generation CAR with suboptimal affinity, such that it barely renders T cell function when activated by single antigen and toxicity is only rescued by ligation of CCR (Kloss et al., 2013). However, this strategy functions by blunting T cell sensitivity to tumor antigen. While this strategy prevents recognition and targeting of single antigen expression tissue, thereby potentially reduced normal tissue toxicity, it also reduces anti-tumor activity. Additionally, the requirement for two antigens to be expressed for efficient T-cell activation and tumor elimination reduces the fraction of tumor capable of CAR activation and increases the potential for the development of tumor escape variants.

An inhibitory CAR (iCAR) fusing specificity for antigen found only on normal tissue, and not on tumor to PD-1 signaling endodomains is capable of significantly inhibiting T-cell-mediated killing and cytokine production in response to binding normal tissue antigen (Fedorov et al., 2013). Impressively, iCAR inhibition of T-cell function is reversible, and T cells are capable of subsequent functionally productive responses upon encounter with tumor antigen. The success of this strategy is dependent of stoichiometry of CAR, iCAR and both antigens. Therefore, it is reasonable to predict that normal tissue toxicity could occur if iCAR expression or antigen is insufficient in the presence of overwhelming CAR/tumor antigen expression. This stoichiometric parameter must evaluated and tightly control for each set of antigens for this strategy to be successful.

Described herein is a method to control T-cell activation to the site of tumor based on the affinity of the scFv used in CAR design to mitigate activation of CAR⁺ T cells in response to low density of EGFR on normal tissue while mediating T-cell cytotoxicity in response to high EGFR density on tumor tissue. Advantages of this method are that (i) reduction of normal tissue toxicity is not associated with mitigated activity in response to tumor and (ii) activation/inhibition of T cells does not require recognition of multiple antigens, for which the stoichiometry of expression and binding to relative receptors must be tightly controlled. Additionally, requiring multiple antigens for T cell activation further reduces the proportion of a tumor that will be efficiently targeted. None of the methods to restrict T-cell on-target, off-tissue tissue toxicity are mutually exclusive, and combinations of multiple strategies may provide improve avoidance of normal tissue destruction.

### E. Clinical Implications

Glioblastoma patients may be an ideal patient population for initial evaluation of safety of T cells specific for EGFR for cancer immunotherapy. EGFR is overexpressed in 40-50% of patients with globlastoma (Parsons et al., 2008; Hu et al., 2013). Additionally, EGFR expression is not reported in normal brain tissue (Yano et al., 2003). Because EGFR is widespread on normal epithelial surfaces, intracavitary delivery of T cells following tumor resection can maximize anti-tumor potential while minimizing the potential for interaction with epithelial surfaces outside of the CNS. Following initial safety evaluation in patients with glioblastoma, it may be possible to extend EGFR-specific CAR⁺ T cell therapy to other EGFR-expressing malignancies, which include breast, ovarian, lung, head and neck, colorectal, and renal cell carcinoma (Hynes et al., 2005).

Although transient expression of CAR through RNA modification of T cells may result in reduced anti-tumor efficacy due to limited presence of CAR⁺ T cells, multiple infusions of RNA-modified T cells, particularly with a weighted initial dose, may overcome these potential limitations, as previously demonstrated with CD19 CAR⁺ T cells modified by RNA transfer in an advanced leukemia murine model (Barrett et al., 2013). While clinical trials with mesothelin-specific CAR transferred by RNA expression have demonstrated the potential for anaphylaxis attributed to the development of IgE antibody responses specific for CAR moieties in response to repeated CAR infusions, a dosing strategy with no more than 10 days between CAR⁺ T cell infusions and treatment to be completed over a course of 21 days has been proposed to avoid isotype switching of IgG antibodies to IgE antibodies and is currently being evaluated (Maus et al., 2013). Despite these challenges, there are many attractive advantage of RNA modification to express CAR in clinical application. First, RNA-modification of T cells does not involve genomic integration of transgenes, and thus have the potential for less cumbersome processes for regulatory approval, which may shorten the preclinical development period for CAR⁺ T cell therapy. In addition, generation of CAR-modified T cells by RNA transfer is much quicker than DNA-modification using the Sleeping Beauty transposon/transposase system, resulting in >90% CAR⁺ T cells in about half of the ex vivo culture time as is required for DNA-modification of T cells. Improving the speed of regulatory approval processes and ex vivo manufacture time could result in getting new CAR⁺ T cell therapies to the clinic faster, quicken the communication time from bench-to-bedside and back to mediate improved efficiency in fine-tuning these therapies for clinical application.

RNA-modification may also provide a platform to test transiently modified T cells specific to widely expressed normal tissue antigens, such as EGFR, in patients to determine safety profiles of CAR structures prior to evaluating permanently integrated CARs as an additional measure of safety. Because Cetux-CAR demonstrates T-cell activation and lytic activity in response to low EGFR density, DNA-modification of T cells to permanently express Cetux-CAR is not likely to be a viable clinical strategy due to the high risk of normal tissue toxicity. However, initial clinical evaluation of Nimo-CAR⁺ T cells modified by RNA transfer may determine the capacity of Nimo-CAR⁺ T cells to mediate normal tissue toxicity with the additional safety feature of transient CAR expression to alleviate concerns of long-term normal tissue toxicity.

While the reduced capacity of Nimo-CAR⁺ T cells to mediate cytotoxicity against low density EGFR functions to reduce normal tissue toxicity, it also may reduce effectiveness against tumors that express low density EGFR, increasing the potential for outgrowth of tumor escape variants expressing EGFR at low density. In contrast, specific lytic activity of Cetux-CAR⁺ T cells against all levels of EGFR expression may reduce the risk of outgrowth of low EGFR expressing tumor escape variants, but does so at the expense of potential toxicity against normal tissue with low EGFR expression. In addition, Cetux-CAR⁺ T cells appear to mediate some degree of T-cell related toxicity independent of targeting normal tissue expressing EGFR, as demonstrated in treatment of intracranial U87 expressing moderate density of EGFR, perhaps due to enhanced cytokine production or induction of local inflammation. The relationship between Cetux-CAR⁺ and Nimo-CAR⁺ T cells highlight the balance that must be achieved between safety and efficacy of gene-modified T cell therapies. Choosing which strategy might have better clinical outcome, Cetux-CAR⁺ T cells with increased risk of toxicity but potential for greater tumor control or Nimo-CAR⁺ T cells with reduced risk of toxicity, but greater potential for development of tumor escape variants, does not have a simple solution. One potential clinical strategy for coping with this balance may be infusing Nimo-CAR⁺ T cell modified by DNA for stable control of high EGFR-expressing tumor variants combined with multiple infusions of Cetux-CAR⁺ T cells modified by RNA to eliminate low EGFR-expressing tumor cells.

### II. Definitions

The term “chimeric antigen receptors (CARs),” as used herein, may refer to artificial T-cell receptors, chimeric T-cell receptors, or chimeric immunoreceptors, for example, and encompass engineered receptors that graft an artificial specificity onto a particular immune effector cell. CARs may be employed to impart the specificity of a monoclonal antibody onto a T cell, thereby allowing a large number of specific T cells to be generated, for example, for use in adoptive cell therapy. In specific embodiments, CARs direct specificity of the cell to a tumor associated antigen, for example. In some embodiments, CARs comprise an intracellular activation domain, a transmembrane domain, and an extracellular domain comprising a tumor associated antigen binding region. In particular aspects, CARs comprise fusions of single-chain variable fragments (scFv) derived from monoclonal antibodies, fused to CD3-zeta a transmembrane domain and endodomain. The specificity of other CAR designs may be derived from ligands of receptors (e.g., peptides) or from pattern-recognition receptors, such as Dectins. In some embodiments, one can target malignant B cells by redirecting the specificity of T cells by using a CAR specific for the B-lineage molecule, CD19. In certain embodiments, the spacing of the antigen-recognition domain can be modified to reduce activation-induced cell death. In certain embodiments, CARs can comprise domains for additional co-stimulatory signaling, such as CD3-zeta, FcR, CD27, CD28, CD137, DAP10, and/or OX40. In some embodiments, molecules can be co-expressed with the CAR, including co-stimulatory molecules, reporter genes for imaging (e.g., for positron emission tomography), gene products that conditionally ablate the T cells upon addition of a pro-drug, homing receptors, chemokines, chemokine receptors, cytokines, and cytokine receptors.

The term “T-cell receptor (TCR)” as used herein refers to a protein receptor on T cells that is composed of a heterodimer of an alpha (α) and beta ( β ) chain, although in some cells the TCR consists of gamma and delta ( γ / δ ) chains. In some embodiments, the TCR may be modified on any cell comprising a TCR, including a helper T cell, a cytotoxic T cell, a memory T cell, regulatory T cell, natural killer T cell, and gamma delta T cell, for example.

As used herein, the term “antigen” is a molecule capable of being bound by an antibody or T-cell receptor. An antigen may generally be used to induce a humoral immune response and/or a cellular immune response leading to the production of B and/or T lymphocytes.

The terms “tumor-associated antigen” and “cancer cell antigen” are used interchangeably herein. In each case, the terms refer to proteins, glycoproteins or carbohydrates that are specifically or preferentially expressed by cancer cells.

As used herein the phrase “in need thereof” with reference to treating a subject or selectively targeting cells in a subject refers to a subject having a disease condition that could benefit from selective killing of cells expressing a target antigen (or an elevated level of a target antigen). In some aspects, the disease condition may be a cancer that expresses an elevated level of a target antigen relative to non-cancerous cells in the subject. For example, the cancer can be a glioma that expresses an elevated level of EGFR relative to non-cancerous cells in the subject.

As used herein the phrase “effective amount” relative to CAR T-cells, or pharmaceutical compositions comprising CAR T-cells, refers to an amount of CAR T-cells that is sufficient, when administered to a subject, to kill cells that express (or express an elevated level of) a target antigen bound by the CAR.

### III. Chimeric Antigen Receptors

Embodiments described herein involve generation and identification of nucleic acids encoding an antigen-specific chimeric antigen receptor (CAR) polypeptide. In some embodiments, the CAR is humanized to reduce immunogenicity (hCAR).

In some embodiments, the CAR may recognize an epitope comprised of the shared space between one or more antigens. Pattern recognition receptors, such as Dectin-1, may be used to derive specificity to a carbohydrate antigen. In certain embodiments, the binding region may comprise complementary determining regions of a monoclonal antibody, variable regions of a monoclonal antibody, and/or antigen binding fragments thereof. In some embodiments the binding region is an scFv. In another embodiment, a peptide (e.g., a cytokine) that binds to a receptor or cellular target may be included as a possibility or substituted for a scFv region in the binding region of a CAR. Thus, in some embodiments, a CAR may be generated from a plurality of vectors encoding multiple scFv regions and/or other targeting proteins. A complementarity determining region (CDR) is a short amino acid sequence found in the variable domains of antigen receptor (e.g., immunoglobulin and T-cell receptor) proteins that complements an antigen and therefore provides the receptor with its specificity for that particular antigen. Each polypeptide chain of an antigen receptor contains three CDRs (CDR1, CDR2, and CDR3). Since the antigen receptors are typically composed of two polypeptide chains, there are six CDRs for each antigen receptor that can come into contact with the antigen -- each heavy and light chain contains three CDRs. Because most sequence variation associated with immunoglobulins and T-cell receptor selectivity are generally found in the CDRs, these regions are sometimes referred to as hypervariable domains. Among these, CDR3 shows the greatest variability as it is encoded by a recombination of the VJ (VDJ in the case of heavy chain and TCR αβ chain) regions.

A CAR-encoding nucleic acid generated via the embodiments may comprise one or more human genes or gene fragments to enhance cellular immunotherapy for human patients. In some embodiments, a full length CAR cDNA or coding region may be generated via the methods described herein. The antigen binding regions or domain may comprise a fragment of the V_{H} and V_{L} chains of a single-chain variable fragment (scFv) derived from a particular human monoclonal antibody, such as those described in U.S. Pat. 7,109,304, incorporated herein by reference. In some embodiments, the scFv comprises an antigen binding domains of a human antigen-specific antibody. In some embodiments, the scFv region is an antigen-specific scFv encoded by a sequence that is optimized for human codon usage for expression in human cells.

The arrangement of the antigen-binding domain of a CAR may be multimeric, such as a diabody or multimers. The multimers can be formed by cross pairing of the variable portions of the light and heavy chains into what may be referred to as a diabody. The hinge portion of the CAR may in some embodiments be shortened or excluded (i.e., generating a CAR that only includes an antigen binding domain, a transmembrane region and an intracellular signaling domain). A multiplicity of hinges may be used with the present embodiments, e.g., as shown in Table 1. In some embodiments, the hinge region may have the first cysteine maintained, or mutated by a proline or a serine substitution, or be truncated up to the first cysteine. The Fc portion may be deleted from scFv used to as an antigen-binding region to generate CARs according to the embodiments. In some embodiments, an antigen-binding region may encode just one of the Fc domains, e.g., either the CH2 or CH3 domain from human immunoglobulin. One may also include the hinge, CH2, and CH3 region of a human immunoglobulin that has been modified to improve dimerization and oligermerization. In some embodiments, the hinge portion of may comprise or consist of an 8-14 amino acid peptide (e.g., a 12 AA peptide), a portion of CD8α, or the IgG4 Fc. In some embodiments, the antigen binding domain may be suspended from cell surface using a domain that promotes oligomerization, such as CD8 alpha. In some embodiments, the antigen binding domain may be suspended from cell surface using a domain that is recognized by monoclonal antibody (mAb) clone 2D3 (mAb clone 2D3 described, e.g., in Singh et al., 2008).

The endodomain or intracellular signaling domain of a CAR can generally cause or promote the activation of at least one of the normal effector functions of an immune cell comprising the CAR. For example, the endodomain may promote an effector function of a T cell such as, e.g., cytolytic activity or helper activity including the secretion of cytokines. The effector function in a naive, memory, or memory-type T cell may include antigen-dependent proliferation. The terms “intracellular signaling domain” or “endodomain” refers to the portion of a CAR that can transduce the effector function signal and/or direct the cell to perform a specialized function. While the entire intracellular signaling domain may be included in a CAR, in some cases a truncated portion of an endodomain may be included. Generally, endodomains include truncated endodomains, wherein the truncated endodomain retains the ability to transduce an effector function signal in a cell.

In some embodiments, an endodomain comprises the zeta chain of the T-cell receptor or any of its homologs (e.g., eta, delta, gamma, or epsilon), MB1 chain, B29, Fc RIII, Fc RI, and combinations of signaling molecules, such as CD3ζ and CD28, CD27, 4-1BB, DAP-10, OX40, and combinations thereof, as well as other similar molecules and fragments. Intracellular signaling portions of other members of the families of activating proteins can be used, such as FcγRIII and FcεRI. Examples of these alternative transmembrane and intracellular domains can be found, e.g., Gross et al. (1992), Stancovski et al. (1993), Moritz et al. (1994), Hwu et al. (1995), Weijtens et al. (1996), and Hekele et al. (1996), which are incorporated herein by reference in their entireties. In some embodiments, an endodomain may comprise the human CD3ζ intracellular domain.

The antigen-specific extracellular domain and the intracellular signaling-domain are preferably linked by a transmembrane domain. Transmembrane domains that may be included in a CAR include, e.g., the human IgG4 Fc hinge and Fc regions, the human CD4 transmembrane domain, the human CD28 transmembrane domain, the transmembrane human CD3ζ domain, or a cysteine mutated human CD3ζ domain, or a transmembrane domains from a human transmembrane signaling protein such as, e.g., the CD16 and CD8 and erythropoietin receptor. Examples of transmembrane domains are provided, e.g., in Table 1.

In some embodiments, the endodomain comprises a sequence encoding a costimulatory receptor such as, e.g., a modified CD28 intracellular signaling domain, or a CD28, CD27, OX-40 (CD134), DAP10, or 4-1BB (CD137) costimulatory receptor. In some embodiments, both a primary signal initiated by CD3 ζ, an additional signal provided by a human costimulatory receptor may be included in a CAR to more effectively activate transformed T cells, which may help improve in vivo persistence and the therapeutic success of the adoptive immunotherapy. As noted in Table 1, the endodomain or intracellular receptor signaling domain may comprise the zeta chain of CD3 alone or in combination with an Fc γ RIII costimulatory signaling domains such as, e.g., CD28, CD27, DAP10, CD137, OX40, CD2, 4-1BB. In some embodiments, the endodomain comprises part or all of one or more of TCR zeta chain, CD28, CD27, OX40/CD134, 4-1BB/CD137, Fc ε RI γ, ICOS/CD278, IL-2Rbeta/CD122, IL-2Ralpha/CD132, DAP10, DAP12, and CD40. In some embodiments, 1, 2, 3, 4 or more cytoplasmic domains may be included in an endodomain. For example, in some CARs it has been observed that at least two or three signaling domains fused together can result in an additive or synergistic effect.

In some aspects, an isolated nucleic acid segment and expression cassette including DNA sequences that encode a CAR may be generated. A variety of vectors may be used. In some preferred embodiments, the vector may allow for delivery of the DNA encoding a CAR to immune such as T cells. CAR expression may be under the control of regulated eukaryotic promoter such as, e.g., the MNDU3 promoter, CMV promoter, EFlalpha promoter, or Ubiquitin promoter. Also, the vector may contain a selectable marker, if for no other reason, to facilitate their manipulation in vitro. In some embodiments, the CAR can be expressed from mRNA in vitro transcribed from a DNA template.

Chimeric antigen receptor molecules are recombinant and are distinguished by their ability to both bind antigen and transduce activation signals via immunoreceptor activation motifs (ITAM’s) present in their cytoplasmic tails. Receptor constructs utilizing an antigen-binding moiety (for example, generated from single chain antibodies (scFv)) afford the additional advantage of being “universal” in that they can bind native antigen on the target cell surface in an HLA-independent fashion. For example, a scFv constructs may be fused to sequences coding for the intracellular portion of the CD3 complex’s zeta chain (ζ), the Fc receptor gamma chain, and sky tyrosine kinase (Eshhar et al., 1993; Fitzer-Attas et al., 1998). Re-directed T cell effector mechanisms including tumor recognition and lysis by CTL have been documented in several murine and human antigen-scFv: ζ systems (Eshhar et al., 1997; Altenschmidt et al., 1997; Brocker et al., 1998).

The antigen binding region may, e.g., be from a human or non-human scFv. One possible problem with using non-human antigen binding regions, such as murine monoclonal antibodies, is reduced human effector functionality and a reduced ability to penetrate into tumor masses. Furthermore, non-human monoclonal antibodies can be recognized by the human host as a foreign protein, and therefore, repeated injections of such foreign antibodies might lead to the induction of immune responses leading to harmful hypersensitivity reactions. For murine-based monoclonal antibodies, this effect has been referred to as a Human AntiMouse Antibody (HAMA) response. In some embodiments, inclusion of human antibody or scFv sequences in a CAR may result in little or no HAMA response as compared to some murine antibodies. Similarly, the inclusion of human sequences in a CAR may be used to reduce or avoid the risk of immune-mediated recognition or elimination by endogenous T cells that reside in the recipient and might recognize processed antigen based on HLA.

In some embodiments, the CAR comprises: a) an intracellular signaling domain, b) a transmembrane domain, c) a hinge region, and d) an extracellular domain comprising an antigen binding region. In some embodiments, the intracellular signaling domain and the transmembrane domain are encoded with the endodomain by a single vector that can be fused (e.g., via transposon-directed homologous recombination) with a vector encoding a hinge region and a vector encoding an antigen binding region. In other embodiments, the intracellular signaling region and the transmembrane region may be encoded by two separate vectors that are fused (e.g., via transposon-directed homologous recombination).

In some embodiments, the antigen-specific portion of a CAR, also referred to as an extracellular domain comprising an antigen binding region, selectively targets a tumor associated antigen. A tumor associated antigen may be of any kind so long as it is expressed on the cell surface of tumor cells. Examples of tumor associated antigens that may be targeted with CARs generated via the embodiments include, e.g., CD19, CD20, carcinoembryonic antigen, alphafetoprotein, CA-125, MUC-1, CD56, EGFR, c-Met, AKT, Her2, Her3, epithelial tumor antigen, melanoma-associated antigen, mutated p53, mutated ras, Dectin-1, and so forth. In some embodiments that antigen specific portion of the CAR is a scFv. Examples of tumor-targeting scFv are provided in Table 1. In some embodiments, a CAR may be co-expressed with a membrane-bound cytokine, e.g., to improve persistence when there is a low amount of tumor-associated antigen. For example, a CAR can be co-expressed with membrane-bound IL-15.

In some embodiments, an intracellular tumor associated antigen such as, e.g., HA-1, survivin, WT1, and p53 may be targeted with a CAR. This may be achieved by a CAR expressed on a universal T cell that recognizes the processed peptide described from the intracellular tumor associated antigen in the context of HLA. In addition, the universal T cell may be genetically modified to express a T-cell receptor pairing that recognizes the intracellular processed tumor associated antigen in the context of HLA.

Additional examples of target antigens for use according to the embodiments include, without limitation CD19, CD20, ROR1, CD22carcinoembryonic antigen, alphafetoprotein, CA-125, 5T4, MUC-1, epithelial tumor antigen, prostate-specific antigen, melanoma-associated antigen, mutated p53, mutated ras, HER2/Neu, folate binding protein, HIV-1 envelope glycoprotein gp120, HIV-1 envelope glycoprotein gp41, GD2, CD123, CD33, CD138, CD23, CD30, CD56, c-Met, meothelin, GD3, HERV-K, IL-11Ralpha, kappa chain, lambda chain, CSPG4, ERBB2, EGFRvIII, VEGFR2, GP240, CD-33, CD-38, VEGFR-1, VEGFR-2, CEA, FGFR3, IGFBP2, IGF-1R, BAFF-R, TACI, APRIL, Fn14, ERBB2 or ERBB35T4, MUC-1, and EGFR. In certain specific aspects, a selected CAR of the embodiments comprises CDRs or the antigen binding portions of nimotuzumab, such as set forth in SEQ ID NOs: 1-2. For example, the CAR can comprise VL CDR1 RSSQNIVHSNGNTYLD (SEQ ID NO: 5); VL CDR2 KVSNRFS (SEQ ID NO: 6); VL CDR3 FQYSHVPWT (SEQ ID NO: 7); VH CDR1 NYYIY (SEQ ID NO: 8); VH CDR2 GINPTSGGSNFNEKFKT (SEQ ID NO: 9) and VH CDR3 QGLWFDSDGRGFDF (SEQ ID NO: 10), see e.g., Mateo et al., 1997, incorporated herein by reference. In further specific aspects, a CAR of the embodiments comprises CDRs or the antigen binding portions of cetuximab, such as set forth in SEQ ID NOs: 3-4. For example, the CAR can comprise VL CDR1 RASQSIGTNIH (SEQ ID NO: 11); VL CDR2 ASEIS (SEQ ID NO: 12); VL CDR3 QQNNNWPTT (SEQ ID NO: 13); VH CDR1 NYGVH (SEQ ID NO: 14); VH CDR2 VIWSGGNTDYNTPFTS (SEQ ID NO: 15) and VH CDR3 ALTYYDYEFAY (SEQ ID NO: 16), see e.g., International (PCT) Pat. Publn. WO2012100346, incorporated herein by reference.

As discussed supra, in some aspects, a selected CAR that binds to an antigen and has a K_{d} of between about 2 nM and about 500 nM relative to the antigen, wherein a T-cell comprising the selected CAR exhibits cytotoxicity to a target cell (e.g., a cancer cell) expressing the antigen. For example, in some aspects, the CAR has a K_{d} of 2, 3, 4, 5, 6, 7, 8, 9, 10, 11, 12, 13, 14, 15, 16, 17, 18, 19 or 20 nM or greater relative to the antigen and a T-cell comprising the selected CAR exhibits cytotoxicity to a target cell expressing the antigen. In still further aspects, the CAR has a K_{d} of between about 5 nM and about 450, 400, 350, 300, 250, 200, 150, 100 or 50 nM relative to the antigen. In still further aspects, the CAR has a K_{d} of between about 5 nM and 500 nM, 5 nM and 200 nM, 5 nM and 100 nM, or 5 nM and 50 nM relative to the antigen and a T-cell comprising the selected CAR exhibits cytotoxicity to a target cell expressing the antigen.

In some aspects, a selected CAR of the embodiments can bind to 2, 3, 4 or more antigen molecules per CAR molecule and a T-cell comprising the selected CAR exhibits cytotoxicity to a target cell (e.g., a cancer cell) expressing the antigen. In some aspects, each to the antigen binding domains of a selected CAR has a K_{d} of 2, 3, 4, 5, 6, 7, 8, 9, 10, 11, 12, 13, 14, 15, 16, 17, 18, 19 or 20 nM or greater relative to the antigen and a T-cell comprising the selected CAR exhibits cytotoxicity to a target cell expressing the antigen. In still further aspects, each to the antigen binding domains of a selected CAR has a K_{d} of between about 5 nM and about 450, 400, 350, 300, 250, 200, 150, 100 or 50 nM relative to the antigen and a T-cell comprising the selected CAR exhibits cytotoxicity to a target cell expressing the antigen. In still further aspects, each to the antigen binding domains of a selected CAR has a K_{d} of between about 5 nM and 500 nM, 5 nM and 200 nM, 5 nM and 100 nM, or 5 nM and 50 nM relative to the antigen and a T-cell comprising the selected CAR exhibits cytotoxicity to a target cell expressing the antigen.

The pathogen recognized by a CAR may be essentially any kind of pathogen, but in some embodiments the pathogen is a fungus, bacteria, or virus. Exemplary viral pathogens include those of the families of Adenoviridae, Epstein-Barr virus (EBV), Cytomegalovirus (CMV), Respiratory Syncytial Virus (RSV), JC virus, BK virus, HSV, HHV family of viruses, Picornaviridae, Herpesviridae, Hepadnaviridae, Flaviviridae, Retroviridae, Orthomyxoviridae, Paramyxoviridae, Papovaviridae, Polyomavirus, Rhabdoviridae, and Togaviridae. Exemplary pathogenic viruses cause smallpox, influenza, mumps, measles, chickenpox, ebola, and rubella. Exemplary pathogenic fungi include Candida, Aspergillus, Cryptococcus, Histoplasma, Pneumocystis, and Stachybotrys. Exemplary pathogenic bacteria include Streptococcus, Pseudomonas, Shigella, Campylobacter, Staphylococcus, Helicobacter, E. coli, Rickettsia, Bacillus, Bordetella, Chlamydia, Spirochetes, and Salmonella. In some embodiments the pathogen receptor Dectin-1 may be used to generate a CAR that recognizes the carbohydrate structure on the cell wall of fungi such as Aspergillus. In another embodiment, CARs can be made based on an antibody recognizing viral determinants (e.g., the glycoproteins from CMV and Ebola) to interrupt viral infections and pathology.

In some embodiments, naked DNA or a suitable vector encoding a CAR can be introduced into a subject’s T cells (e.g., T cells obtained from a human patient with cancer or other disease). Methods of stably transfecting T cells by electroporation using naked DNA are known in the art. See, e.g., U.S. Pat. No. 6,410,319. Naked DNA generally refers to the DNA encoding a chimeric receptor of the embodiments contained in a plasmid expression vector in proper orientation for expression. In some embodiments, the use of naked DNA may reduce the time required to produce T cells expressing a CAR generated via methods of the embodiments.

Alternatively, a viral vector (e.g., a retroviral vector, adenoviral vector, adeno-associated viral vector, or lentiviral vector) can be used to introduce the chimeric construct into T cells. Generally, a vector encoding a CAR that is used for transfecting a T cell from a subject should generally be non-replicating in the subject’s T cells. A large number of vectors are known that are based on viruses, where the copy number of the virus maintained in the cell is low enough to maintain viability of the cell. Illustrative vectors include the pFB-neo vectors (STRATAGENE®) as well as vectors based on HIV, SV40, EBV, HSV, or BPV.

Once it is established that the transfected or transduced T cell is capable of expressing a CAR as a surface membrane protein with the desired regulation and at a desired level, it can be determined whether the chimeric receptor is functional in the host cell to provide for the desired signal induction. Subsequently, the transduced T cells may be reintroduced or administered to the subject to activate anti-tumor responses in the subject. To facilitate administration, the transduced T cells may be made into a pharmaceutical composition or made into an implant appropriate for administration in vivo, with appropriate carriers or diluents, which are preferably pharmaceutically acceptable. The means of making such a composition or an implant have been described in the art (see, for instance, Remington’s Pharmaceutical Sciences, 16th Ed., Mack, ed. (1980)). Where appropriate, transduced T cells expressing a CAR can be formulated into a preparation in semisolid or liquid form, such as a capsule, solution, injection, inhalant, or aerosol, in the usual ways for their respective route of administration. Means known in the art can be utilized to prevent or minimize release and absorption of the composition until it reaches the target tissue or organ, or to ensure timed-release of the composition. Generally, a pharmaceutically acceptable form is preferably employed that does not ineffectuate the cells expressing the chimeric receptor. Thus, desirably the transduced T cells can be made into a pharmaceutical composition containing a balanced salt solution such as Hanks’ balanced salt solution, or normal saline.

### IV. Methods and Compositions Related to the Embodiments

In certain aspects, the embodiments described herein include a method of making and/or expanding the antigen-specific redirected T cells that comprises transfecting T cells with an expression vector containing a DNA construct encoding the hCAR, then, optionally, stimulating the cells with antigen positive cells, recombinant antigen, or an antibody to the receptor to cause the cells to proliferate.

In another aspect, a method is provided of stably transfecting and redirecting T cells by electroporation, or other non-viral gene transfer (such as, but not limited to sonoporation) using naked DNA. Most investigators have used viral vectors to carry heterologous genes into T cells. By using naked DNA, the time required to produce redirected T cells can be reduced. “Naked DNA” means DNA encoding a chimeric T-cell receptor (cTCR) contained in an expression cassette or vector in proper orientation for expression. An electroporation method according to the embodiments produces stable transfectants that express and carry on their surfaces the chimeric TCR (cTCR).

In certain aspects, the T cells are primary human T cells, such as T cells derived from human peripheral blood mononuclear cells (PBMC), PBMC collected after stimulation with G-CSF, bone marrow, or umbilical cord blood. Conditions include the use of mRNA and DNA and electroporation. Following transfection the cells may be immediately infused or may be stored. In certain aspects, following transfection, the cells may be propagated for days, weeks, or months ex vivo as a bulk population within about 1, 2, 3, 4, 5 days or more following gene transfer into cells. In a further aspect, following transfection, the transfectants are cloned and a clone demonstrating presence of a single integrated or episomally maintained expression cassette or plasmid, and expression of the chimeric receptor is expanded ex vivo. The clone selected for expansion demonstrates the capacity to specifically recognize and lyse CD19 expressing target cells. The recombinant T cells may be expanded by stimulation with IL-2, or other cytokines that bind the common gamma-chain (e.g., IL-7, IL-12, IL-15, IL-21, and others). The recombinant T cells may be expanded by stimulation with artificial antigen presenting cells. The recombinant T cells may be expanded on artificial antigen presenting cell or with an antibody, such as OKT3, which cross links CD3 on the T cell surface. Subsets of the recombinant T cells may be deleted on artificial antigen presenting cell or with an antibody, such as Campath, which binds CD52 on the T cell surface. In a further aspect, the genetically modified cells may be cryopreserved.

T-cell propagation (survival) after infusion may be assessed by: (i) q-PCR using primers specific for the CAR; (ii) flow cytometry using an antibody specific for the CAR; and/or (iii) soluble TAA.

Embodiments described hereinalso concern the targeting of a B-cell malignancy or disorder including B cells, with the cell-surface epitope being CD19-specific using a redirected immune T cell. Malignant B cells are an excellent target for redirected T cells, as B cells can serve as immunostimulatory antigen-presenting cells for T cells. Preclinical studies that support the anti-tumor activity of adoptive therapy with donor-derived CD19-specific T-cells bearing a human or humanized CAR include (i) redirected killing of CD19⁺ targets, (ii) redirected secretion/expression of cytokines after incubation with CD19⁺ targets/stimulator cells, and (iii) sustained proliferation after incubation with CD19⁺ targets/stimulator cells.

In certain embodiments, the CAR cells are delivered to an individual in need thereof, such as an individual that has cancer or an infection. The cells then enhance the individual’s immune system to attack the respective cancer or pathogenic cells. In some cases, the individual is provided with one or more doses of the antigen-specific CAR T-cells. In cases where the individual is provided with two or more doses of the antigen-specific CAR T-cells, the duration between the administrations should be sufficient to allow time for propagation in the individual, and in specific embodiments the duration between doses is 1, 2, 3, 4, 5, 6, 7, or more days.

The source of the allogeneic T cells that are modified to include both a chimeric antigen receptor and that lack functional TCR may be of any kind, but in specific embodiments the cells are obtained from a bank of umbilical cord blood, peripheral blood, human embryonic stem cells, or induced pluripotent stem cells, for example. Suitable doses for a therapeutic effect would be at least 10⁵ or between about 10⁵ and about 10¹⁰ cells per dose, for example, preferably in a series of dosing cycles. An exemplary dosing regimen consists of four one-week dosing cycles of escalating doses, starting at least at about 10⁵ cells on Day 0, for example increasing incrementally up to a target dose of about 10¹⁰ cells within several weeks of initiating an intra-patient dose escalation scheme. Suitable modes of administration include intravenous, subcutaneous, intracavitary (for example by reservoir-access device), intraperitoneal, and direct injection into a tumor mass.

A pharmaceutical composition of the embodiments can be used alone or in combination with other well-established agents useful for treating cancer. Whether delivered alone or in combination with other agents, a pharmaceutical composition of the embodiments can be delivered via various routes and to various sites in a mammalian, particularly human, body to achieve a particular effect. One skilled in the art will recognize that, although more than one route can be used for administration, a particular route can provide a more immediate and more effective reaction than another route.

A composition of the embodiments can be provided in unit dosage form wherein each dosage unit, e.g., an injection, contains a predetermined amount of the composition, alone or in appropriate combination with other active agents. The term unit dosage form as used herein refers to physically discrete units suitable as unitary dosages for human and animal subjects, each unit containing a predetermined quantity of the composition of the embodiments, alone or in combination with other active agents, calculated in an amount sufficient to produce the desired effect, in association with a pharmaceutically acceptable diluent, carrier, or vehicle, where appropriate. The specifications for the novel unit dosage forms of the embodiments depend on the particular pharmacodynamics associated with the pharmaceutical composition in the particular subject.

Desirably an effective amount or sufficient number of the isolated transduced T cells is present in the composition and introduced into the subject such that long-term, specific, anti-tumor responses are established to reduce the size of a tumor or eliminate tumor growth or regrowth than would otherwise result in the absence of such treatment. Desirably, the amount of transduced T cells reintroduced into the subject causes a 10%, 20%, 30%, 40%, 50%, 60%, 70%, 80%, 90%, 95%, 98%, or 100% decrease in tumor size when compared to otherwise same conditions wherein the transduced T cells are not present.

Accordingly, the amount of transduced T cells administered should take into account the route of administration and should be such that a sufficient number of the transduced T cells will be introduced so as to achieve the desired therapeutic response. Furthermore, the amounts of each active agent included in the compositions described herein (e.g., the amount per each cell to be contacted or the amount per certain body weight) can vary in different applications. In general, the concentration of transduced T cells desirably should be sufficient to provide in the subject being treated at least from about 1 × 10⁶ to about 1 × 10⁹ transduced T cells, even more desirably, from about 1 × 10⁷ to about 5 × 10⁸ transduced T cells, although any suitable amount can be utilized either above, e.g., greater than 5 × 10⁸ cells, or below, e.g., less than 1 × 10⁷ cells. The dosing schedule can be based on well-established cell-based therapies (see, e.g., Topalian and Rosenberg, 1987; U.S. Pat. No. 4,690,915), or an alternate continuous infusion strategy can be employed.

These values provide general guidance of the range of transduced T cells to be utilized by the practitioner upon optimizing the methods of the embodiments. The recitation herein of such ranges by no means precludes the use of a higher or lower amount of a component, as might be warranted in a particular application. For example, the actual dose and schedule can vary depending on whether the compositions are administered in combination with other pharmaceutical compositions, or depending on interindividual differences in CAR-expressing cells (e.g., CAR binding affinity to a target antigen). One skilled in the art readily can make any necessary adjustments in accordance with the exigencies of the particular situation.

### V. Antigen Presenting Cells

In some cases, APCs are useful in preparing CAR-based therapeutic compositions and cell therapy products. APCs for use according to the embodiments include but arte not milted to dendritic cells, macrophages and artificial antigen presenting cells. For general guidance regarding the preparation and use of antigen-presenting systems, see, e.g., U.S. Pat. Nos. 6,225,042, 6,355,479, 6,362,001 and 6,790,662; U.S. Pat. Application Publication Nos. 2009/0017000 and 2009/0004142; and International Publication No. WO2007/103009).

APCs may be used to expand T Cells expressing a CAR. During encounter with tumor antigen, the signals delivered to T cells by antigen-presenting cells can affect T-cell programming and their subsequent therapeutic efficacy. This has stimulated efforts to develop artificial antigen-presenting cells that allow optimal control over the signals provided to T cells (Turtle et al., 2010). In addition to antibody or antigen of interest, the APC systems may also comprise at least one exogenous assisting molecule. Any suitable number and combination of assisting molecules may be employed. The assisting molecule may be selected from assisting molecules such as co-stimulatory molecules and adhesion molecules. Exemplary co-stimulatory molecules include CD70 and B7.1 (also called B7 or CD80), which can bind to CD28 and/or CTLA-4 molecules on the surface of T cells, thereby affecting, e.g., T-cell expansion, Thl differentiation, short-term T-cell survival, and cytokine secretion such as interleukin (IL)-2 (see Kim et al., 2004). Adhesion molecules may include carbohydrate-binding glycoproteins such as selectins, transmembrane binding glycoproteins such as integrins, calcium-dependent proteins such as cadherins, and single-pass transmembrane immunoglobulin (Ig) superfamily proteins, such as intercellular adhesion molecules (ICAMs), that promote, for example, cell-to-cell or cell-to-matrix contact. Exemplary adhesion molecules include LFA-3 and ICAMs, such as ICAM-1. Techniques, methods, and reagents useful for selection, cloning, preparation, and expression of exemplary assisting molecules, including co-stimulatory molecules and adhesion molecules, are exemplified in, e.g., U.S. Pat. Nos. 6,225,042, 6,355,479, and 6,362,001.

Cells selected to become aAPCs, preferably have deficiencies in intracellular antigen-processing, intracellular peptide trafficking, and/or intracellular MHC Class I or Class II molecule-peptide loading, or are poikilothermic (i.e., less sensitive to temperature challenge than mammalian cell lines), or possess both deficiencies and poikilothermic properties. Preferably, cells selected to become aAPCs also lack the ability to express at least one endogenous counterpart (e.g., endogenous MHC Class I or Class II molecule and/or endogenous assisting molecules as described above) to the exogenous MHC Class I or Class II molecule and assisting molecule components that are introduced into the cells. Furthermore, aAPCs preferably retain the deficiencies and poikilothermic properties that were possessed by the cells prior to their modification to generate the aAPCs. Exemplary aAPCs either constitute or are derived from a transporter associated with antigen processing (TAP)-deficient cell line, such as an insect cell line. An exemplary poikilothermic insect cells line is a Drosophila cell line, such as a Schneider 2 cell line (e.g., Schneider, J.m 1972). Illustrative methods for the preparation, growth, and culture of Schneider 2 cells, are provided in U.S. Pat. Nos. 6,225,042, 6,355,479, and 6,362,001.

APCs may be subjected to a freeze-thaw cycle. For example, APCs may be frozen by contacting a suitable receptacle containing the APCs with an appropriate amount of liquid nitrogen, solid carbon dioxide (dry ice), or similar low-temperature material, such that freezing occurs rapidly. The frozen APCs are then thawed, either by removal of the APCs from the low-temperature material and exposure to ambient room temperature conditions, or by a facilitated thawing process in which a lukewarm water bath or warm hand is employed to facilitate a shorter thawing time. Additionally, APCs may be frozen and stored for an extended period of time prior to thawing. Frozen APCs may also be thawed and then lyophilized before further use. Preservatives that might detrimentally impact the freeze-thaw procedures, such as dimethyl sulfoxide (DMSO), polyethylene glycols (PEGs), and other preservatives, may be advantageously absent from media containing APCs that undergo the freeze-thaw cycle, or are essentially removed, such as by transfer of APCs to media that is essentially devoid of such preservatives.

In other embodiments, xenogenic nucleic acid and nucleic acid endogenous to the aAPCs may be inactivated by crosslinking, so that essentially no cell growth, replication or expression of nucleic acid occurs after the inactivation. For example, aAPCs may be inactivated at a point subsequent to the expression of exogenous MHC and assisting molecules, presentation of such molecules on the surface of the aAPCs, and loading of presented MHC molecules with selected peptide or peptides. Accordingly, such inactivated and selected peptide loaded aAPCs, while rendered essentially incapable of proliferating or replicating, may retain selected peptide presentation function. The crosslinking can also result in aAPCS that are essentially free of contaminating microorganisms, such as bacteria and viruses, without substantially decreasing the antigen-presenting cell function of the aAPCs. Thus crosslinking can be used to maintain the important APC functions of aAPCs while helping to alleviate concerns about safety of a cell therapy product developed using the aAPCs. For methods related to crosslinking and aAPCs, see for example, U.S. Pat. Application Publication No. 20090017000, which is incorporated herein by reference.

### VI. Kits

Any of the compositions described herein may be comprised in a kit. In some embodiments, allogeneic CAR T-cells are provided in the kit, which also may include reagents suitable for expanding the cells, such as media, antigen presenting cells (e.g., aAPCs), growth factors, antibodies (e.g., for sorting or characterizing CAR T-cells) and/or plasmids encoding CARs or transposase.

In a non-limiting example, a chimeric receptor expression construct, one or more reagents to generate a chimeric receptor expression construct, cells for transfection of the expression construct, and/or one or more instruments to obtain allogeneic cells for transfection of the expression construct (such an instrument may be a syringe, pipette, forceps, and/or any such medically approved apparatus).

In some embodiments, an expression construct for eliminating endogenous TCR α/β expression, one or more reagents to generate the construct, and/or CAR⁺ T cells are provided in the kit. In some embodiments, there includes expression constructs that encode zinc finger nuclease(s).

In some aspects, the kit comprises reagents or apparatuses for electroporation of cells.

The kits may comprise one or more suitably aliquoted compositions of the embodiments or reagents to generate compositions of the embodiments. The components of the kits may be packaged either in aqueous media or in lyophilized form. The container means of the kits may include at least one vial, test tube, flask, bottle, syringe, or other container means, into which a component may be placed, and preferably, suitably aliquoted. Where there is more than one component in the kit, the kit also will generally contain a second, third, or other additional container into which the additional components may be separately placed. However, various combinations of components may be comprised in a vial. The kits of the embodiments also will typically include a means for containing the chimeric receptor construct and any other reagent containers in close confinement for commercial sale. Such containers may include injection or blow molded plastic containers into which the desired vials are retained, for example.

### VII. EXAMPLES

The following specific and non-limiting examples are to be construed as merely illustrative, and do not limit the present disclosure in any way whatsoever. Without further elaboration, it is believed that one skilled in the art can, based on the description herein, utilize the present disclosure to its fullest extent. All publications cited herein are hereby incorporated by reference in their entirety. Where reference is made to a URL or other such identifier or address, it is understood that such identifiers can change and particular information on the internet can come and go, but equivalent information can be found by searching the internet. Reference thereto evidences the availability and public dissemination of such information.

### Example 1 - Materials and Methods

### Plasmids

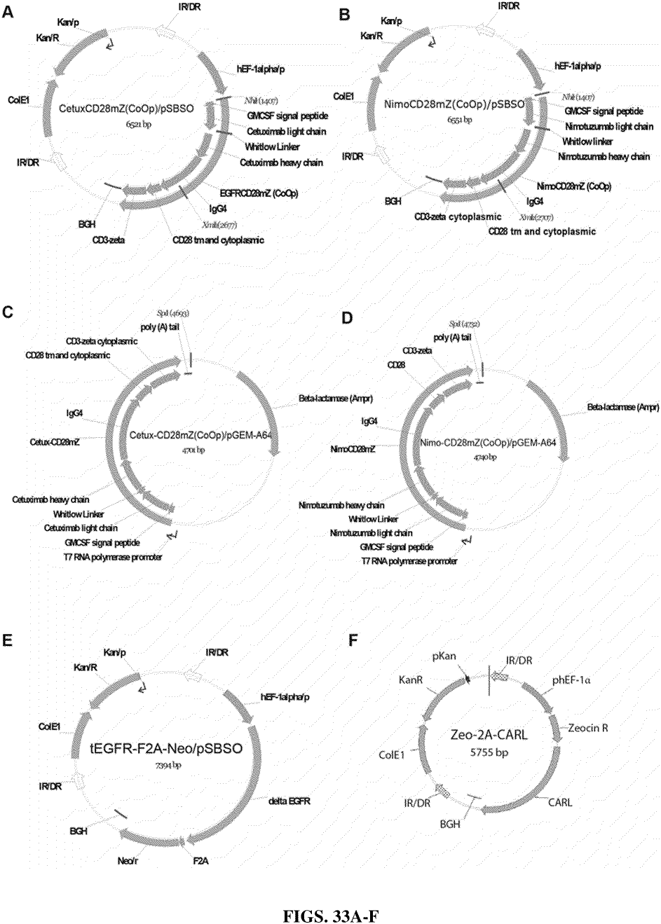

Cetuximab-derived CAR transposon. Cetuximab-derived CAR is composed of the following: a signal peptide from human GMCSFR2 signal peptide (amino acid 1-22; NP_758452.1), variable light chain of cetuximab (PDB:1YY9_C) whitlow linker (AAE37780.1), variable heavy chain of cetuximab (PDB:1YY9_D), human IgG4 (amino acids 161-389, AAG00912.1), human CD28 transmembrane and signaling domains (amino acids 153-220, NP_006130), and human CD3-ζ intracellular domain (amino acids 52 through 164, NP_932170.1). Sequence of GMCSFR2, variable light chain, whitlow linker, variable heavy chain and partial IgG4 were human codon optimized and generated by GeneART (Regensburg, Germany) as 0700310/pMK. Previously described CD19CD28mZ(CoOp)/pSBSO under control of human elongation factor 1-alpha (HEF1α) promoter was selected as backbone for SB transposon. 0700310/pMK and previously described CD19CD28mZ/pSBSO (93, 94) underwent double digestion with NheI and XmnI restriction enzymes. CAR insert and transposon backbone were identified as DNA fragments of 1.3 kb and 5.2 kb, respectively, by agarose gel electrophoresis in a 0.8% agarose gel run at 150 volts for 45 minutes and stained with ethidium bromide for visualization under ultraviolet light exposure. Bands were excised and purified (Qiaquick Gel Extraction kit, Qiagen, Valencia, CA), then ligated using T4 DNA ligase (Promega, Madison, WI) at a molar ratio of insert to backbone of 3:1. Heat shock transformation of TOP10 chemically competent bacteria (Invitrogen, Grand Island, NY) and selection on kanamycin-containing agar plates cultured at 37° C. for 12-16 hours identified bacteria clones positive for transposon backbone. Six clones were selected for mini-culture in TB media with kanamycin selection at 37° C. for 8 hours. Preparation of DNA from mini-cultures was done via MiniPrep kit (Qiagen) and subsequent analytical digestion with restriction enzymes and analysis of fragment size by agarose gel electrophoresis identified clones positive for CetuxCD28mZ (CoOp)/pSBSO (FIG. 33A). Positive clone was inoculated 1:1000 into large culture in TB media with kanamycin antibiotic selection and cultured on shaker at 37° C. for 16 hours, until log-phase growth was achieved. DNA was isolated from bacteria using EndoFree Maxi Prep kit (Qiagen). Spectrophotometer analysis of DNA verified purity by OD260/280 reading between 1.8 and 2.0.

Nimotuzumab-derived CAR transposon. Nimotuzumab-derived CAR is composed of the following: a signal peptide from human GMCSFR2 signal peptide (amino acids 1-19, NP_001155003.1), variable light chain of nimotuzumab (PDB:3GKW_L) whitlow linker (GenBank: AAE37780.1), variable heavy chain of nimotuzumab (PDB:3GKW_H), human IgG4 (amino acids 161-389, AAG00912.1), human CD28 transmembrane and signaling domains (amino acids 153-220, NP_006130), and human CD3-ζ intracellular domain (amino acids 52 through 164, NP_932170.1). Sequence of GMCSFR2, variable light chain, whitlow linker, variable heavy chain and partial IgG4 were human codon optimized and generated by GeneART as 0841503/pMK. 08541503/pMK and previously described CD19CD28mZ/pSBSO (Singh et al., 2013; Singh et al., 2008) underwent double digestion with NheI and XmnI restriction enzymes, ligation, transformation, large scale amplification and purification of plasmid NimoCD28mZ(CoOp)/pSBSO (FIG. 33B) were performed as described above.

SB11 transposase. The hyperactive SB11 transposase under control of CMV promoter (Kan-CMV-SB11) was used as previously described (Singh et al., 2008; Davies et at., 2010).

pGEM/GFP/A64. GFP under control of of a T7 promoter followed by 64 A-T base pairs and a SpeI site was use to in vitro transcribe GFP RNA. The cloning of pGEM/GFP/A64 has been previously described (Boczkowski et al., 2000).

Cetuximab-derived CAR/pGEM-A64. Cetuximab-derived CAR was cloned into an intermediate vector, pSBSO-MCS, by NheI and XmnI double digestion of CetuxCD28mZ(CoOp)/pSBSO and CD19CD28mZ(CoOp)/pSBSO-MCS. Cetux-CAR insert and pSBSO-MCS backbone were isolated by extraction from agarose gel after electrophoresis and ligated, transformed, and amplified on large-scale as described in generation of CetuxCD28mZ(CoOp)/pSBSO. CetuxCD28mZ(CoOp) was cloned into pGEM/GFP/A64 plasmid to place Cetux-CAR under control of a T7 promoter for in vitro transcription of RNA with artificial poly-A tail 64 nucleotides in length. CetuxCD28mZ(CoOp)/pSBSO-MCS was digested with NheI and EcoRV at 37° C. while pGEM/GFP/A64 was sequentially digested with XbaI at 37° C. then SmaI at 25° C. Digested Cetux-CAR insert and pGEM/A64 backbone were separated by electrophoresis in 0.8% agarose gel run at 150 volts for 45 minutes and visualized by ethidium bromide staining and UV light exposure. Fragments were excised from gel and purified by Qiaquick Gel Extraction (Qiagen) and ligated using T4 DNA ligase (Promega) at 3:1 insert to vector molar ratio and incubated at 16° C. overnight. Dam -/- C2925 chemcially competent bacteria (Invitrogen) were transformed by heat shock and cultured overnight at 37° C. on ampicillin-containing agar for selection of clones containing pGEM/A64 backbone. Eight clones were selected for small-scale DNA amplification by inoculation in TB media with ampicillin antibiotic selection and cultured on a shaker at 37° C. for 8 hours. Purification of DNA was performed using MiniPrep kit (Qiagen) and analytical restriction enzyme digest and subsequent electrophoresis determined which clones expressed correct ligation product, CetuxCD28mZ/pGEM-A64 (FIG. 33C). A positive clone was selected an inoculated 1:1000 in TB containing ampicillin. After 18 hours of culture at 37° C., DNA was purified using EndoFree Plasmid Purification kit (Qiagen). Spectrophotometry analysis confirmed high quality DNA by OD260/280 ration between 1.8 and 2.0.

Nimotuzumab-derived CAR/pGEM-A64. NimoCD28mZ(CoOp)/pSBSO was digested sequentially with NheI at 37° C. and SfiI at 50° C. while pGEM/GFP/A64 was digested sequentially with XbaI at 37° C. and SfiI at 50° C. NimoCD28mZ(CoOp) was cloned into pGEM/GFP/A64 plasmid to place Nimo-CAR under control of a T7 promoter for in vitro transcription of RNA with artificial poly A tail 64 nucleotides in length. Digested Nimo-CAR insert and pGEM/A64 backbone were separated by electrophoresis in 0.8% agarose gel run at 150 volts for 45 minutes and visualized by ethidium bromide staining and UV light exposure. Fragments were excised from gel and purified by Qiaquick Gel Extractions (Qiagen) and ligated using T4 DNA ligase (Promega) at 3:1 insert to vector molar ratio and incubated at 16° C. overnight. Dam-/- C2925 chemically competent bacteria (Invitrogen) were transformed by heat shock and cultured overnight at 37° C. on ampicillin-containing agar for selection of clones containing pGEM/A64 backbone. Eight clones were selected for small-scale DNA amplification by inoculation in TB media with ampicillin antibiotic selection and cultured on a shaker at 37° C. for 8 hours. Purification of DNA was performed using MiniPrep kit (Qiagen) and analytical restriction enzyme digest and subsequent electrophoresis determined which clones expressed correct ligation product, NimoCD28mZ/pGEM-A64 (FIG. 33D). A positive clone was selected an inoculated 1:1000 in TB containing ampicillin. After 18 hours of culture at 37° C., DNA was purified using EndoFree Plasmid Purification kit (Qiagen). Spectrophotometry analysis confirmed high quality DNA by OD260/280 ration between 1.8 and 2.0.

Truncated EGFR transposon. Truncated EGFR was cloned into a SB transposon linked via self-cleavable peptide sequence F2A to a gene for neomycin resistance. A codon-optimized truncated form of human EGFR (accession NP_005219.2) containing only extracellular and transmembrane domains, 0909312 ErbB1/pMK-RQ, was synthesized by GeneArt (Regensburg, Germany). ErbB1/pMK-RQ was digested with NheI and SmaI at 37° C. while tCD19-F2A-Neo/pSBSO was sequentially digested with NheI at 37° C., then NruI at 37° C. with a purification step between (Qiaquick Gel Extraction kit, Qiagen). tEGFR insert and F2A-Neo/pSBSO backbone were separated by gel electrophoresis on 0.8% agarose gel run at 150 volts for 45 minutes. Bands of predicted sizes were isolated (Qiaquick Gel Extraction kit, Qiagen) and ligated with T4 DNA Ligase (Promega) overnight at 16° C. TOP10 chemically competent cells (Invitrogen) were heat-shock transformed with ligation production and cultured overnight on agar containing kanamycin. Five clones were inoculated for small scale DNA amplification by culture in TB containing kanamycin for 8 hours. DNA purification by Mini Prep kit (Qiagen) and subsequent analytical restriction enzyme digest identified clones positive for tErbB1-F2A-Neo/pSBSO (FIG. 33E). A positive clone was inoculated into culture at 1:1000 for large-scale DNA amplification at cultured on a shaker at 37° C. for 16 hours. Purification of DNA from bacteria in log-phase growth was performed using EndoFree Plasmid Purification kit (Qiagen) and spectrophotometry verified DNA purity by OD 260/280 reading between 1.8 and 2.0.

CAR-L transposon. A previously described 2D3 hybridoma (94) was used to derive the scFv sequence of CAR-L. Briefly, RNA was extracted from hybridoma by RNeasy Mini Kit (Qiagen), according to manufacturer’s instructions. Reverse transcription via Superscript III First Strand kit (Invitrogen) generated a cDNA library. PCR using degenerate primers for the FR1 region amplified mouse variable heavy and light chains, which were subsequently ligated into TOPO TA vector. CAR-L was constructed as a codon optimized sequence, as follows: Following a human GMCSFR signal peptide (amino acid 1-22; NP_758452.1), 2D3-derived scFv was fused to human CD8α extracellular domain (amino acid 136-182; NP_001759.3) and transmembrane and intracellular domains of human CD28 (amino acid 56-123; NP_001230006.1) and terminates in human intracellular domain of CD3ζ (amino acid. 48-163; NP_ 000725.1). The CAR-L protein was synthesized at GeneArt, then excised and ligated into a SB transposon with a self-cleavable 2A peptide fused to a Zeomycin resistance gene, designated CAR-L-2A-Zeo (FIG. 33F) (Rushworth et al., 2014).

### Cell Lines: Propagation and Modification

All cell lines were maintained in complete media Dulbecco’s modified eagle media (DMEM) (Life Technologies, Grand Island, NY), supplemented with 10% heat inactivated fetal bovine serum (FBS) (HyClone, ThermoScientific) and 2 mM Glutamax-100 (Gibco, Life Technologies) at 5% CO2, 95% humidity and 37° C., unless otherwise noted. Adherent cell lines were routinely cultured to 70-80% confluency, then passaged 1:10 following dissociation with 0.05% Trypsin-EDTA (Gibco). Identity of cell lines was validated by STR DNA fingerprinting using the AmpF_STR Identifier kit according to manufacturer’s instructions (Applied Biosystems, cat# 4322288). The STR profiles were compared to known ATCC fingerprints (ATCC.org), and to the Cell Line Integrated Molecular Authentication database (CLIMA) version 0.1.200808 (on the world wide web at bioinformatics.istge.it/clima/) (Nucleic Acids Research 37:D925-D932 PMCID: PMC2686526). The STR profiles matched known DNA fingerprints.

OKT3-loaded K562 clone 4. K562 clone 4 was received as a gift from Carl June, M.D. at the University of Pennsylvania and has been previously described (Suhoski et al., 2007; Paulos et al., 2008). Clone 4 are modified to express tCD19, CD86, CD137L, CD64 and a membrane IL15-GFP fusion protein and have been manufactured as a working cell bank for pre-clinical and clinical studies under PACT. K562 clone 4 can be made to express anti-CD3 antibody, OKT3, through binding to the CD64 high affinity Fc receptor. To load OKT3 onto K562 clone 4, cells are cultured overnight in X-VIVO serum free media (Lonza, Cologne, Germany) with 1× 20% N-Acetylcysteine at a density of 1×10⁶ cells/mL. This step clears the Fc receptors for optimal binding of OKT3. The following day, cells are washed and resuspended at 1×10⁶ cells/mL in X-VIVO media with 1× 20% N-Acetylcysteine and irradiated at achieve 100 Gy. Cells are washed and resuspended at 1×10⁶ cells/mL in PBS and OKT3 (eBioscience, San Diego, CA) is added at a concentration of 1 mg/mL and incubated on roller at 4° C. for 30 minutes. Cells are washed again, stained to verify expression of costimulatory molecules and OKT3 by flow cytometry, and cryopreserved.

tEGFR⁺ K562 clone 27. K562 clone 27 was derived from K562 clone 9, gift from Carl June, M.D. at the University of Pennsylvania. K562 clone 9 was lentivirally transduced, as previously described (Suhoski et al., 2007; Paulos et al., 2008), to express tCD19, CD86, CD137L, and CD64. Clone 27 were modified from clone 9 to stably express a membrane tethered IL15-IL15Rα fusion protein (Hurton, L. V., 2014) via SB transfection, cloned by limiting dilution, and verified to have high expression of all transgenes by flow cytometry. K562 clone 27 was modified to express truncated EGFR by SB transfection of tErbB1-F2A-Neo/pSBSO. K562 clone 27 expressing EGFR were incubated with PE-labeled EGFR-specific antibody (BD Biosciences, Carlsbad, CA, cat# 555997) and anti-PE beads (Miltenyi Biotec, Auburn, CA), then separated from non-labeled cells by flow through a magnetic column (Miltenyi Biotec). Following magnetic selection, tEGFR⁺ K562 clone 27 were cultured in the presence of 1 mg/mL G418 (Invivogen, San Diego, CA) to maintain high EGFR expression.

EL4, CD19⁺ EL4, tEGFR⁺ EL4, and CAR-L⁺ EL4. EL4 were obtained from ATCC and modified to express tCD19-F2A-Neo, tEGFR-F2A-Neo or CAR-L-F2A-Neo by SB non-viral gene modification. EL4 were electroporated in using Amaxa Nucelofector (Lonza) and primary mouse T cell kit (Lonza) according to manufacturer’s instructions. Briefly, 2×10⁶ EL4 cells were centrifuged at 90xg for 10 minutes and resuspended in 100 uL primary mouse T cell buffer with 3 µg transposon (tCD19-F2A-Neo, tEGFR-F2A-Neo, or CAR-L-2A-Zeo) and 2 ug SB11 transposase and electroporated using Amaxa program X-001. Following electroporation, cells were immediately transferred to pre-warmed and supplemented primary mouse T cell media, supplied with kit (Lonza). The following day, 1 mg/mL G418 was added to select for EL4 cells modified to express transgenes. Expression was verified by flow cytometry 7 days post-modification.

U87, U87low, U87med, and U87high. U87, formally designated U87MG, were obtained from ATCC (Manassas, VA). U87low and U87med were generated to overexpress EGFR by electroporation with tErbB1-F2A-Neo/pSBSO and SB11 using Amaxa Nucleofector and cell line Nucleofector kit T (Lonza, cat#VACA-1002), according to manufacturer’s instructions. Briefly, U87 cells were cultured to 80% confluency, then harvested by dissociation in 0.05% Trypsin-EDTA (Gibco) and counted via trypan blue exclusion using and automated cell counter (Cellometer, Auto T4 Cell Counter, Nexcelcom, Lawrence, MA).1x10⁶ U87 cells were suspended in 100 µL cell line kit T electroporation buffer in the presence of 3 µg of tErbB1-F2A-Neo/pSBSO transposon and 2 µg SB11 transposase, transferred to a cuvette and electroporated via program U-029. Immediately following electroporation, cells were transferred to 6-well plate and allowed to recover in complete DMEM media. The following day, 0.35 mg/mL G418 (Invivogen) was added to select for transgene expression. After propagation to at least 1x10⁶ cells, flow cytometry was performed to assess EGFR expression. Electroporated U87 cells demonstrated modest increase in EGFR expression relative to unmodified U87 and were designated U87low. To generate U87med cells, U87 cells were lipofectamine-transferred with tErbB1-F2A-Neo and SB11 using Lipofectamine 2000 (Invitrogen) according to manufacturer’s instructions. The following day, 0.35 mg/mL G418 was added to culture to select for neomycin resistance. After propagation of cells to significant number, flow cytometrey revealed a two-peak population, with mutually exclusive modest or high EGFR overexpression, relative to U87 cells. Cells were stained with anti-EGFR-PE and FACS sorted for the top 50% of highest peak. Careful subcloning when cells reached no greater than 70% confluence and flow cytometry analysis was routinely performed to ensure cells maintained EGFR expression. U87high are U87-172b cells overexpressing wtEGFR, and were a kind gift from Oliver Bölger, Ph.D.

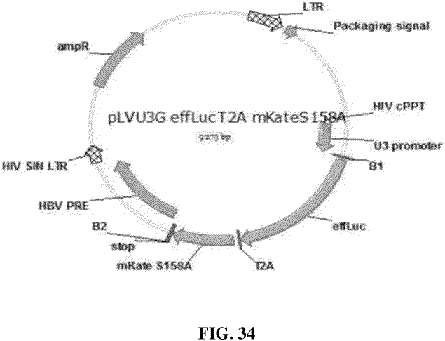

U87-ffLuc-mKate and U87med-ffLuc-mKate. U87 and U87med cells were lentivirally transduced to express ffLuc-mKate transgene (FIG. 34), similar to a previously described protocol (Turkman et al., 2011). Briefly, 293-METR packaging cells were transfected with pcMVR8.2, VSV-G and pLVU3GeffLuc-T2AmKates158A in the presence of Lipofectamine 2000 (Invitrogen), according to manufacturer’s instructions. After 48 hours, virus-like particles (VLP) were harvested and concentrated on 100 kDa NMWL filters (Millipore, Billerica, MA). To transduce U87 and U87med, cells were plated in 6 well plates until 70-80% confluent, then ffLucmKate VLPs were added in conjunction with 8 µg/mL polybrene. The plate was centrifuged at 1800 rpm for 1.5 hours, then incubated for 6 hours. Following incubation, supernatant was removed. Twenty-four hours after transduction, cells reached confluency and were subcultured and FACS sorted for cells expressing moderate levels of ffLuc-mKate.

Human renal cortical epithelial cells (HRCE). HRCE were obtained from Lonza, described to be taken from proximal and distal renal tubules of healthy individuals, and were cultured in complete Renal Growth Media (Lonza, cat# CC-3190) supplemented with recombinant human epidermal growth factor (rhEGFR), epinephrine, insulin, triiodothyronine, hydrocortisone, transferrin, 10% heat-inactivated FBS (HyClone), and 2 mM Glutamax-100 (Gibco). HRCE have finite lifespan in vitro, therefore, all assays were performed with cells that underwent less than 10 population doublings. Cells were cultured to 70-80% confluency, then detached by 0.05% Trypsin-EDTA (Gibco) and passaged 1:5 in fresh, complete Renal Growth Media.

NALM-6, T98G, LN18 and A431. NALM-6, T98G, LN18, and A431 were all obtained from ATCC and cultured as described for cell lines.

T cell modification and culture. Peripheral blood mononuclear cells were obtained from healthy donors from Gulf Coast Regional Blood Bank and isolated by Ficoll-Paque (GE Healthcare, Milwaukee, WI) and cryopreserved. All T cell cultures were maintained in complete RPMI-1640 (HyClone), supplemented with 10% FBS (HyClone) and 2 mM Glutamax (Gibco).

Electroporation with SB Transposon/Transposase. SB electroporation was performed as previously described (Singh et al., 2008). PBMC were thawed on the day of electroporation and rested in cytokine-free media complete RPMI-1640 at a density of 1x10⁶ cells/mL for 2 hours. Following resting period, cells were centrifuged at 200xg for 8 minutes, then resuspended in media and counted by trypan blue exclusion using an automated cell counter (Cellometer, Auto T4 Cell Counter, Nexcelcom). PBMC were centrifuged again and resuspended at 2×10⁸ /mL in human T cell electroporation buffer (Lonza, cat# VPA-1002), then 100 µL of cell suspension was mixed with 15 µg transposon (either Cetux- or Nimo-CAR) and 5 µg SB11 transposase, transferred to electroporation cuvette, and electroporated via Amaxa Nucleofector (Lonza) using program U-014 for unstimulated human T cells. Following electroporation, cells were immediately transferred to phenol-free RPMI supplemented with 20% heat-inactivated FBS (HyClone), and 2 mM Glutamax-100 (Gibco) to recover overnight. The next day, cells were analyzed by flow cytometry for CD3 and Fc (to determine CAR expression) to determine transient expression of transposon.

Stimulation and Culture of CAR⁺ T cells. Twenty-four hours after electroporation, cells were stimulated with 100 Gy-irradiated EGFR⁺ K562 clone 27 artificial antigen presenting cells (aAPC) at a ratio of 2 CAR⁺ T cells:1 aAPC. T cells were restimulated every 7-9 days following evaluation of CAR expression by flow cytometry. Throughout culture period, T cells received 30 ng/mL IL-21 (Peprotech, Rocky Hill, NJ) added to culture every 2-3 days. IL-2 (Aldeleukin, Novartis, Switzerland) was added to culture after second stimulation cycle at 50 U/mL, every 2-3 days. At day 14, cultures were evaluated for the presence of NK cells, designated as CD3^{neg}CD56⁺ cells present in culture. If NK cells represented >10% of cell population, NK cell depletion was performed by labeling NK cells with CD56-specific magnetic beads (Miltenyi Biotec) and sorting on LS column (Miltenyi Biotec). Flow cytometry of negative flow through containing CAR⁺ T cells verified successful depletion of NK cell subset from culture. Cultures were evaluated for function when CAR was expressed on >85% of CD3⁺ T cells, usually following 5 stimulation cycles.

In vitro transcription of RNA. CetuxCD28mZ/pGEM-A64, NimoCD28mZ/pGEM-A64, or GFP/pGEM-A64 was digested with SpeI at 37° C. for 4 hours to provide linear template for in vitro RNA transcription. Complete linearization of template confirmed by agarose gel electrophoresis in 0.8% agarose gel and presence of single band and remaining digest purified by QiaQuick PCR Purification (Qiagen) and eluted in low volume to achieve concentration of 0.5 µg/µL. In vitro transcription reaction was performed using T7 mMACHINE mMESSAGE Ultra (Ambion, Life Technologies, cat# AM1345) according to manufacturer’s protocol and incubated at 37° C. for 2 hours. After transcription of mRNA, DNA template was degraded by addition of supplied Turbo DNAse at 1 unit/µg DNA template and incubated an additional 30 minutes at 37° C. Transcribed RNA was purified using RNeasy Mini kit (Qiagen). Concentration and purity (OD 260/280 value = 2.0-2.2) were determined by spectrophotometry and frozen in single-thaw aliquots at -80° C. Quality of RNA product evaluated by gel electrophoresis on formaldehyde-containing agarose gel (1% agarose, 10% 10x MOPS Running Buffer, 6.7% formaldehyde) at 75 volts for 80 minutes in 1xMOPS Running Buffer and visualization of single, delineated band.

Polyclonal T-cell expansion. Numeric expansion of T cells independent of antigen was achieved by culture with 100 Gy-irradiated K562 clone 4 loaded with OKT3 delivering proliferative stimulus through cross-linking CD3. aAPC were added at a density of 10:1 or 1:2 T cells: aAPC every 7-10 days, 50 U/mL IL-2 was added every 2-3 days. Media changes were performed throughout culture to keep T cells at a density between 0.5-2×10⁶ cells/mL.

RNA electro-transfer to T cells. T cells underwent stimulation 3-5 days prior to RNA transfer by co-culture with 100 Gy-irradiated OKT3-loaded K562 clone 4 as described above. Prior to electro-transfer, T cells were harvested and counted by trypan blue exclusion using an automated cell counter (Cellometer, Auto T4 Cell Counter, Nexcelcom). During preparation of cells, RNA was removed from -80° C. freezer and thawed on ice. T cells were centrifuged at 90xg for 10 minutes, and supernatant was carefully aspirated to ensure complete removal without disruption of cell pellet. T cells were suspended in P3 Primary Cell 4D-Nucleofector buffer (Lonza, cat # V4XP-3032) to a concentration of 1×10⁸/mL and 20 µL of each T cell suspension was mixed with 3 µg of in vitro transcribed RNA, then transferred to Nucleofector cuvette strip (Lonza, cat # V4XP-3032). Cells were electroporated in Amaxa 4D Nucleofector (Lonza) using program DQ-115, then allowed to rest in cuvette up to 15 minutes. Following rest period, warm recovery media, phenol-free RPMI 1640 (HyClone) supplemented with 2 mM Glutamax-100 (Gibco) and 20% heat-inactivated FBS (HyClone), was added to cuvette and cells were gently transferred to 6 well plate containing recovery media and transferred to a tissue culture incubator. After 4 hours, 50 U/mL IL-2 and 30 ng/mL IL-21 were added to the T cells. Four to twenty-four hours after RNA transfer, T cells were analyzed for expression of CAR by flow cytometry for Fc. All functional assays were carried out at 24 hours post-RNA transfer.

### Immunostaining and Flow Cytometry

Acquisition and analysis. Flow cytometry data were collected on FACS Calibur (BD Biosciences, San Jose, CA) and acquired using CellQuest software (version 3.3, BD Biosciences). Analysis of flow cytometry data was performed using FlowJo software (version x.0.6, TreeStar, Ashland, OR).

Surface Immunostaining and Antibodies. Immunostaining of up to 1x10⁶ cells was performed with monoclonal antibodies conjugated to the following dyes at the following dilutions (unless otherwise stated): fluorescein (FITC, 1:25), phycoerythrin (PE, 1:40), peridinin chlorophyll protein conjugated to cyanine dye (PerCPCy5.5, 1:25), allophycocyanin (APC, 1:40), AlexaFluor488 (1:20), AlexaFluor647 (1:20). All antibodies were purchased from BD Biosciences, unless otherwise stated. Antibodies specific for the following were used: CD3 (clone SK7), CD4 (clone RPA-T4), CD8 (clone SK1), CD19 (HIB19), CD27 (clone L128), CD28 (clone L293), CD45RA (clone HI100), CD45RO (clone HI100), CD56 (clone B159), CD62L (clone DREG-56), CCR7 (clone GD43H7, Biolegend, San Diego, CAR PerCPCy5.5 diluted 1:45), EGFR (clone EGFR.1, PE diluted 1:13.3), Fc (to detect CAR, clone HI10104, Invitrogen), IL15 (clone 34559, R&D Systems, Minneapolis, MN, PE diluted 1:20), murine F(ab′)2 (to detect OKT3 loaded on K562, Jackson Immunoresearch, West Grove, PA, cat# 115-116-072, PE diluted 1:100), TNF-α (clone mAb11, PE diluted 1:40) and IFN-γ (clone 27, APC diluted 1:66.7), pErk1/2 (clone 20A, AlexaFluor 647), pp38 (clone 36/p38, PE) and Ki-67 (clone B56, FITC, 1:20, BD Biosciences). Surface molecules were stained in FACS buffer (PBS, 2% FBS, 0.5% sodium azide) for 30 minutes in the dark at 4° C.

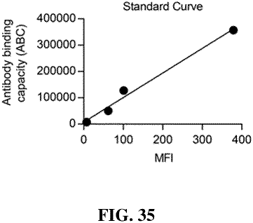

Quantitative Flow Cytometry. Quantitative flow cytometry was performed using Quantum Simply Cellular polystyrene beads (Bangs Laboratories, Fishers, IN). Five bead populations are provided, four populations with increasing amounts of anti-murine IgG, and therefore a known antibody binding capacity (ABC) and one blank population. EGFR-PE (BD Biosciences, cat#555997) was incubated with beads at a saturated concentration (1:3 dilution, per manufacturer’s recommendation) synchronously with immunostaining of target cells. MFI of EGFR-PE binding to microspheres was used to create a standard curve, to which a linear regression was fit using QuickCal Data Analysis Program (version 2.3, Bangs Laboratories) (FIG. 35). Applying measured MFI of EGFR-PE binding to target cells, less the amount of background autofluroescence, to the linear regression yielded a mean number of EGFR molecules expressed per cell.

Intracellular cytokine staining and flow cytometry. T cells were co-cultured with target cells at a ratio of 1:1 for 4-6 hours in the presence of GolgiStop diluted 4000x (BD Biosciences). Unstimulated T cells served as negative controls, while T cells treated with Leukocyte Activation Cocktail, containing PMA/Ionomycin and brefeldin A (BD Biosciences) diluted 1000x served as positive controls. An EGFR-specific monoclonal antibody (clone LA1, Millipore) was used to block interaction of CAR and EGFR interaction. Intracellular cytokine staining was performed after surface immunostaining by fixation/permeabilization in Cytofix/Cytoperm buffer (BD Biosciences) for 20 minutes in the dark at 4° C., followed by staining of intracellular cytokine in 1x Perm/Wash Buffer (BD Biosciences) for 30 minutes, in the dark at 4° C. Antibodies used were TNF-α (BD Biosciences, clone mAb11, PE diluted 1:40) and IFN-γ (BD Biosciences, clone 27, APC diluted 1:66.7). Following intracellular cytokine staining, cells were fixed with 0.5% paraformaldehyde (CytoFix, BD Biosciences) until samples were acquired on FACS Calibur.

Measuring phosphorylation by flow cytometry. T cells were co-cultured with target cells at a ratio of 1:1 for 45 minutes, unless otherwise indicated. Following activation, T cells centrifuged 300xg for 5 min and supernatant decanted. T cells were lysed and fixed by addition of 20 volumes of 1x PhosFlow Lyse/Fix buffer (BD Biosciences), pre-warmed to 37° C. and incubated at 37° C. for 10 minutes. Following centrifugation, T cells are permeabilized by addition of ice-cold PhosFlow Perm III Buffer (BD Biosciences) while vortexing and incubated on ice in the dark for 20 minutes. After incubation, cells were washed with FACS Buffer and resuspended in 100 µL staining solution. Staining solution was composed of antibodies against CD4 (clone SK3, FITC), CD8 (clone SK1, PerCPCy5.5), pErk1/2 (clone 20A, AlexaFluor 647), pp38 (clone 36/p38, PE) and FACS buffer, all present at the same ratio and incubated for 20 minutes in the dark at room temperature. Cells were fixed with 0.5% paraformaldehyde and analyzed by flow cytometry within 24 hours.

Viability Staining. Staining for Annexin V (BD Biosciences) and 7-AAD (BD Biosciences) was used to determine cell viability and was performed in 1x Annexin Binding buffer, with staining for CD4 or CD8, for 20 minutes, in the dark, at room temperature. Percentage of viable cells was determined as %AnnexinV^{neg}7-AAD^{neg} in CD4 or CD8 gated T cell population.

Staining for cellular proliferation marker Ki-67. Proliferation marker Ki-67 was measured by intracellular flow cytometry. T cells were co-cultured with adherent target cells at a ratio of 1:5 for 36 hours, then T cells were harvested from culture by removing supernatant and centrifugation at 300xg. T cells were then fixed and permeabilized by drop-wise addition of ice-cold 70% ethanol while vortexing at high speed. T cells were then stored at -20° C. for 2-24 hours before staining. Cells were stained with Ki-67 (clone B56, FITC, 1:20, BD Biosciences), CD4 (clone RPA-T4), and CD8 (clone SK1) in 100 µL FACs Buffer for 30 min in the dark at room temperature, then immediately analyzed by flow cytometry.

### T-cell Functional Assays

CAR downregulation. CAR⁺ T cells and targets were harvested and counted by trypan blue exclusion using an automated cell counter (Cellometer, Auto T4 Cell Counter, Nexcelcom), then mixed at a 1:1 ratio in a 12-well plate, and individual wells were harvested at each time point to measure CAR surface expression on T cells. Negative controls for downregulation were T cells plated without stimuli. Staining for T cells by CD3, CD4 and CD8 expression and co-staining for CAR by Fc was analyzed on flow cytometer. Percent downregulation of CAR was calculated as [CAR expression following stimuli]/[CAR expression without stimuli] x 100.

Secondary activation and cytokine production. CAR⁺ T cells and adherent targets were harvested and counted by trypan blue exclusion using an automated cell counter (Cellometer, Auto T4 Cell Counter, Nexcelcom), then mixed at a ratio of 1:1 in a 12-well plate. After 24 hours of co-culture, T cells were harvested from culture by removing supernatant and washing adherent cells with PBS. T cells were spun at 300xg for 5 minutes, then resuspended in media and counted by trypan blue exclusion using an automated cell counter (Cellometer, Auto T4 Cell Counter, Nexcelcom). T cells were stimulated with targets at 1:1 ratio and intracellular cytokine production analysis as described above.

Long-term cytotoxicity assay. The day prior to initiation of assay, adherent U87 and U87high cells were harvested, counted, and 40,000 target cells were plated in each well of a 6-well plate in complete DMEM and incubated in tissue culture incubator overnight. On the day of assay, CAR⁺ T cells were harvested, counted by trypan blue exclusion, and added at a 1:5 E:T ratio to plated target cells. Negative control wells had no T cells added. At each assay time point, T cells were removed by discarding supernatant and washing the well with PBS. Adherent cells were dissociated from wells by 0.05% Trypsin-EDTA (Gibco). Microscopy was performed to visually ensure complete detachment of cells from well. Harvested cells were spun down and resuspended in 100 µL of media, then counted by trypan blue exclusion using a hemacytometer. Percent surviving cells was calculated as [cell number after T cell co-culture]/[cell number with no T cell co-culture] x 100.

Chromium release assay. Specific cytotoxicity was assessed via standard 4 hour chromium release assay, as previously described (Singh et al., 2008). Target cells were harvested and counted by trypan blue exclusion using an automated cell counter (Cellometer, Auto T4 Cell Counter). No less than 250,000 cells were aliquoted, then centrifuged at 300xg for 5 minutes and supernatant was discarded. Next, 0.1 µCi of 51Cr was added to each target and incubated for 1-1.5 hours in a tissue culture incubator at 37° C. 100,000 T cells per well were plated in triplicate and serially diluted at 1:2 ratio to give a final effector to target (E:T) ratio of 20:1, 10:1, 5:1, 2.5:1 and 1.25:1 in a 96-well V-bottom plate (Corning, Corning, NY) and placed in a tissue culture incubator. Media only was placed in wells for minimum chromium release control. Following labeling with chromium, targets were washed three times with 10 mL PBS, then resuspended at a final concentration of 125,000 cells/mL, thoroughly mixed, and 100 µL was added to each row, included all T-cell containing rows, a minimum release row, and a maximum release row. Plates were centrifuged at 300xg for 3 minutes. Following centrifugation, 100 µL of 0.1% Triton X-100 (Sigma-Aldrich, St. Louis, MO) was added to maximum release row, and plates were placed in tissue culture incubator for 4 hours. Following incubation, plates were then harvested by careful removal of 50 µL supernatant, without disrupting cell pellet, and transferred to LumaPlate-96 (Perkin-Elmer, Waltham, MA) and allowed to dry overnight. The following day, plates were sealed with Top-Seal (Perkin-Elmer) and scintillation measured on TopCount NXT (Perkin-Elmer). Percent specific lysis was calculated as [(51Cr released - minimum) / (maximum- minimum)] x 100 where maximum and minimum values were averaged for each triplicate.

High-throughput gene expression and CDR3 sequencing

Analysis of gene expression by direct imaging of mRNA transcripts. Direct imaging and quantification of mRNA molecules was performed as previously described (319-322). Cells prior to or following expansion were positively sorted for CD4 and CD8 expression by incubating with CD4 and CD8 magnetics beads (Miltenyi Biotec), respectively, and sorting on LS column. Flow cytometry was used to verify purity of CD4 and CD8 separated populations. 1x10⁶ T cells were lysed in 165 pL of RLT Buffer (Qiagen) and frozen at -80° C. in single-thaw aliquots. RNA lysates were thawed and hybridized with multiplexed target-specific, color-coded reporter and biotinylated capture probes at 65° C. for 12 hours. Lymphocyte specific mRNA transcripts of interest were identified and two CodeSets generated from RefSeq accessions were used to generate reporter and capture probe pairs, a Lymphocyte CodeSet, and TCR Va and Vβ CodeSet. The Lymphocyte CodeSet contained probes for the following genes: ABCB1; ABCG2; ACTB; ADAM19; AGER; AHNAK; AIF1; AIM2; AIMP2; AKIP1; AKT1; ALDH1A1; ANXA1; ANXA2P2; APAF1; ARG1; ARRB2; ATF3; ATM; ATP2B4; AXIN2; B2M; B3GAT1; BACH2; BAD; BAG1; BATF; BAX; BCL10; BCL11B; BCL2; BCL2L1; BCL2L1; BCL2L11; BCL2L11; BCL6; BCL6B; BHLHE41; BID; BIRC2; BLK; BMI1; BNIP3; BTLA; C21orf33; CA2; CA9; CARD9; CASP1; CAT; CBLB; CCBP2; CCL3; CCL4; CCL5; CCNB1; CCND1; CCR1; CCR2; CCR4; CCR5; CCR6; CCR7; CD160; CD19; CD19R-scfv; CD19RCD28; CD2; CD20-scfv rutuximab); CD226; CD244; CD247; CD27; CD274; CD276; CD28; CD300A; CD38; CD3D; CD3E; CD4; CD40LG; CD44; CD45R-scfv; CD47; CD56R-scfv; CD58; CD63; CD69; CD7; CD80; CD86; CD8A; CDH1; CDK2; CDK4; CDKN1A; CDKN1B; CDKN2A; CDKN2C; CEBPA; CFLAR; CFLAR; CHPT1; CIITA; CITED2; CLIC1; CLNK; c-MET-scfv; CREB1; CREM; CRIP1; CRLF2; CSAD; CSF2; CSNK2A1; CTGF; CTLA4; CTNNA1; CTNNB1; CTNNBL1; CTSC; CTSD; CX3CL1; CX3CR1; CXCL10; CXCL12; CXCL9; CXCR1; CXCR3; CXCR4; DAPL1; DEC1; DECTIN-1R; DGKA; DOCKS; DOK2; DPP4; DUSP16; EGFR-scfv (NIMO CAR); EGLN1; EGLN3; EIF1; ELF4; ELOF1; ENTPD1; EOMES; EPHA2; EPHA4; EPHB2; ETV6; FADD; FAM129A; FANCC; FAS; FASLG; FCGR3B; FGL2; FLT1; FLT3LG; FOS; FOXO1; FOXO3; FOXP1; FOXP3; FYN; FZD1; G6PD; GABPA; GADD45A; GADD45B; GAL3ST4; GAS2; GATA2; GATA3; gBAD-1R-scfv; GEMIN2; GFI1; GLIPR1; GLO1; GNLY; GSK3B; GZMA; GZMB; GZMH; HCST; HDAC1; HDAC2; HER2-scfv; HERV-K 6H5-scfv; HLA-A; HMGB2; HOPX; HOXA10; HOXA9; HOXB3; HOXB4; HPRT1; HRH1; HRH2; Human CD19R-scfv; ICOS; ICOSLG; ID2; ID3; IDOl; IFNA1; IFNG; IFNGR1; IGF1R; IKZF1; IKZF2; IL10; IL10RA; IL12A; IL12B; IL12RB1; IL12RB2; IL13; IL15; IL15RA; IL17A; IL17F; IL17RA; IL18; IL18R1; IL18RAP; IL1A; IL1B; IL2; IL21R; IL22; IL23A; IL23R; IL27; IL2RA; IL2RB; IL2RG; IL4; IL4R; IL5; IL6; IL6R; IL7R; IL9; IRF1; IRF2; IRF4; ITCH; ITGA1; ITGA4; ITGA5; ITGAL; ITGAM; ITGAX; ITGB1; ITGB7; ITK; JAK1; JAK2; JAK3; JUN; JUNB; KIR2DL1; KIR2DL2; KIR2DL3; KIR2DL4; KIR2DL5A; KIR2DS1; KIR2DS2; KIR2DS3; KIR2DS4; KIR2DS5; KIR3DL1; KIR3DL2; KIR3DL3; KIR3DS1; KIT; KLF10; KLF2; KLF4; KLF6; KLF7; KLRAP1; KLRB1; KLRC1; KLRC2; KLRC3; KLRC4; KLRD1; KLRF1; KLRG1; KLRK1; LAG3; LAIR1; LAT; LAT2; LCK; LDHA; LEF1; LGALS1; LGALS3; LIFR; LILRB1; LOC282997; LRP5; LRP6; LRRC32; LTA; LTBR; LYN; MAD1Ll; MAP2K1; MAPK14; MAPK3; MAPK8; MBD2; MCL1; MIF; MMP14; MPL; MTOR; MXD1; MYB; MYC; MYO6; NANOG; NBEA; NCAM1; NCL; NCR1; NCR2; NCR3; NCRNA00185; NEILl; NEIL2; NFAT5; NFATC1; NFATC2; NFATC3; NFKB1; NOS2; NOTCH1; NR3C1; NR4A1; NREP; NRIP1; NRP1; NT5E; OAZ1; OPTN; P2RX7; PAX5; PDCD1; PDCD1LG2; PDE3A; PDE4A; PDE7A; PDK1; PDXK; PECAM1; PHACTR2; PHC1; POLR1B; POLR2A; POP5; POUSF1; PPARA; PPP2R1A; PRDM1; PRF1; PRKAA2; PRKCQ; PROM1; PTGER2; PTK2; PTPN11; PTPN4; PTPN6; PTPRK; RAB31; RAC1; RAC2; RAF1; RAP1GAP2; RARA; RBPMS; RHOA; RNF125; RORA; RORC; RPL27; RPS13; RUNX1; RUNX2; RUNX3; S100A4; S100A6; SATB1; SCML1; SCML2; SEL1L; SELL; SELPLG; SERPINE2; SH2B3; SH2D2A; SIT1; SKAP1; SKAP2; SLA2; SLAMF1; SLAMF7; SLC2A1; SMAD3; SMAD4; SNAIl; SOCS1; SOCS3; SOD1; SOX13; SOX2; SOX4; SOX5; SPIl; SPN; SPRY2; STAT1; STAT3; STAT4; STAT5A; STAT5B; STAT6; STMN1; SYK; TAL1; TBP; TBX21; TBXA2R; TCF12; TCF3; TCF7; TDGF1; TDO2; TEK; TERF1; TERT; TF; TFRC; TGFA; TGFB1; TGFB2; TGFBR1; Thymidine Kinase; TIE1; TLR2; TLR8; TNF; TNFRSF14; TNFRSF18; TNFRSF1B; TNFRSF4; TNFRSF9; TNFSF10; TNFSF11; TNFSF14; TOX; TP53; TRAF1; TRAF2; TRAF3; TSC22D3; TSLP; TXK; TYK2; TYROBP; UBASH3A; VAX2; VEGFA; WEE1; XBP1; XBP1; YY1AP1; ZAP70; ZBTB16; ZC2HC1A; ZEB2; ZNF516. The TCR Va and Vβ CodeSet contained probes for the following genes: TRAV1-1; TRAV1-2; TRAV2; TRAV3; TRAV4; TRAV5; TRAV6; TRAV7; TRAV8-1; TRAV8-2; TRAV8-3; TRAV8-6; TRAV9-1; TRAV9-2; TRAV10; TRAV11; TRAV12-1; TRAV12-2; TRAV12-3; TRAV13-1; TRAV13-2; TRAV14; TRAV16; TRAV17; TRAV18; TRAV19; TRAV20; TRAV21; TRAV22; TRAV23; TRAV24; TRAV25; TRAV26-1; TRAV26-2; TRAV27; TRAV29; TRAV30; TRAV34; TRAV35; TRAV36; TRAV38-1; TRAV38-2; TRAV39; TRAV40; TRAV41; TRBV2; TRBV3-1; TRBV4-1; TRBV4-2; TRBV4-3; TRBV5-1; TRBV5-4; TRBV5-5; TRBV5-6; TRBV5-8; TRBV6-1; TRBV6-2; TRBV6-4; TRBV6-5; TRBV6-6; TRBV6-8; TRBV6-9; TRBV7-2; TRBV7-3; TRBV7-4; TRBV7-6; TRBV7-7; TRBV7-8; TRBV7-9; TRBV9; TRBV10-1; TRBV10-2; TRBV10-3; TRBV11-1; TRBV11-2; TRBV11-3; TRBV12-3; TRBV12-5; TRBV13; TRBV14; TRBV15; TRBV16; TRBV18; TRBV19; TRBV20-1; TRBV24-1; TRBV25-1; TRBV27; TRBV28; TRBV29-1; TRBV30. Following hybridization, samples were processed in nCounter Prep (NanoString Technologies, Seattle, WA), and analyzed in nCounter Digital Analyzer (NanoString Technologies). Reference genes were identified that span wide range of RNA expression levels: ACTB, G6PD, OA21, POLR1B, RPL27, RPS13, and TBP and were used to normalize data. Normalization to positive-, negative-, and house-keeping genes was using nCounter RCC Collector (version 1.6.0, NanoString Technologies). A statistical test developed for digital gene expression profiling was used to determine differential expression of genes between sample pairs (O′Connor et al., 2012; Audic et al., 1997). After normalization, significant differential gene expression in the Lymphocyte CodeSet was identified by a combination of p<0.01 and a fold change greater than 1.5 in at least ⅔ pairs, as previously described (O′Connor et al., 2012). Heat-mapping of normalized values for differentially RNA transcripts was performed by hierarchical clustering and TreeView software, version 1.1 (Eisen et al., 1998). After normalization, percentage of TCR Vα and Vβ were derived from count data as previously described (Zhang et al., 2012).

High-throughput CDR3 deep-sequencing. TCRβ CDR3 regions were amplified and sequenced from DNA extracted from 1x10⁶ T cells (Qiagen DNeasy Blood and Tissue Kit, Qiagen) and carried out on ImmunoSEQ platform (Adaptive Technologies, Seattle, WA), as previously described (Robins et al., 2009).

In vivo evaluation of T cells in intracranial glioma xenograft murine model

All animal experiments were carried out under guidance and regulation from the Institutional Animal Care and Use Committee (IACUC) at MD Anderson Cancer Center under the approved animal protocol ACUF 11-11-13131. All mice used were 7-8 week old female NOD.Cg-PrkdescidIL2Rγtm1Wjl/Sz strain (NSG) (Jackson Laboratory, Bar Harbor, ME).

Implantation of guide-screw. Mice aged 7-8 weeks were anesthetized using ketamine/xylazine cocktail (10 mg/mL ketamine, 0.5 mg/mL xylazine) dosed at 0.1 mL/10 g. Implantation of guide-screw was performed as previously described (Lal et al., 2000) Once unresponsive to stimuli, surgical area on head was prepared by shaving fur and treated with povidone-iodine (polyvinylpyrrolidone complexed with elemental iodine) antiseptic solution. Using surgically ascpetic technique, a 1 cm incision was made down the middle of the cranium. An opening was made using a 1 mm drill bit (DH#60, Plastics One, Roanoke, VA) extending 1 mm from drill (DH-0, Plastics One) using firm circular pressure. A guide-screw (Plastics One, cat # C212SG) with a 0.50 mm opening in the center and a 1.57 mm shaft diameter was inserted into the drill site using a screwdriver (SD-80, Plastics One). Incision sites were sutured and mice were given 0.01 mg/mL buprenorphine dosed at 0.1 mL/10 grams as post-surgical analgesic. Mice recovered from surgery on low-power heat source until full mobility was regained.

Implantation of U87-ffLucm-Kate or U87med-ffLuc-mKate tumor cells. Mice recovered from guide-screw implantation for 2-3 weeks before intracranial tumors were established, as previously described (Lal et al., 2000). U87-ffLuc-mKate or U87med-ffLuc-mKate were dissociated from tissue culture vessel following 10 minute incubation with Cell Dissociation Buffer, enzyme-free, PBS (Gibco) at room temperature. Cells were counted by trypan blue exclusion using hemacytometer and centrifuged at 200xg for 8 minutes. Following centrifugation, cells were resuspended in sterile PBS to a final concentration of 50,000 cells/µL. Mice were anesthetized with isoflurane (2-chloro-2-(difluoromethoxy)-1,1,1-trifluoro-ethane), and prepared for incision as described above. While mice were undergoing surgical preparation, 26 gauge, 10 µL Hamilton syringes with blunt needle (Hamilton Company, Reno, NV cat# 80300) were prepared by placing plastic guard 2.5 mm from the end of syringe and loading 5 µL of cell suspension containing 250,000 cells. After incision site was opened, syringes were inserted into guide screw opening and cells were injected with constant slow pressure. After completion of injection, syringes were held in place an additional 30 seconds to allow intracranial pressure to dissipate, then slowly removed. Incisions were sutured and mice were removed from isoflurane exposure. Day of implantation is designated as day 0 of study. On day 1 and 4 tumors were imaged via non-invasive bioluminescent imaging, as described above to ensure successful tumor engraftment. Mice were then divided into three groups to evenly distribute relative tumor flux, and then randomly assigned to receive Cetux-CAR⁺ T-cell treatment, Nimo-CAR⁺ T-cell treatment and no treatment.

Non-invasive bioluminescent imaging of U87-ffLuc-mKate or U87med-ffLuc-mKate. Intracranial glioma was non-invasively and serially imaged and used as a measure of relative tumor burden. Ten minutes after sub-cutaneous injection of 215 µg D-luciferin potassium salt (Caliper Life Sciences, Perkin-Elmer), tumor flux (photons/s/cm2/steradian) was measured using Xenogen Spectrum (Caliper Life Sciences, Perkin-Elmer) and Living Image software (version 2.50, Caliper Life Sciences, Perkin-Elmer). Tumor flux was measured in a delineated region of interest encompassing entire cranial region of mice.

Delivery of CAR⁺ T cells to intracranially established U87-ffLuc-mKate or U87med-ffLuc-mKate glioma. Treatment of intracranial glioma xenografts began on day 5 of tumor establishment and continued weekly for a total of 3 T cell injections. CAR⁺ T cells having completed 3 stimulation cycles were confirmed to be >85% CAR-expressing by flow cytometry, then viable cells were counted by trypan blue exclusion using an automated cell counter (Cellometer, Auto T4 Cell Counter, Nexcelcom). CAR⁺ T cells were spun at 300xg for 5 minutes, and resuspended at a concentration of 0.6×10⁶ /µL in sterile PBS. Mice were prepared for cranial incision as described above, and anesthetized by isoflurane exposure. While mice were being prepared, 26 gauge, 10 µL Hamilton syringes with blunt needle (Hamilton Company, cat# 80300) were prepared by placing plastic guard 2.5 mm from the end of syringe and loading 5 µL of cell suspension containing 3×10⁶ T cells. Syringes were inserted into the guide-screw, extending 2.5 mm into intracranial space, and injected with slow, constant pressure. After syringe was emptied, it was held in place an addition 30 seconds to allow intracranial pressure to dissipate. Following injection, incisions were sutured closed and mice were removed from isoflurane exposure.

Assessing survival of mice. Mice were sacrificed when they displayed progressive weight loss (>25% of body mass), rapid weight loss (>10% loss of body mass within 48 hours) or hind limb paralysis, or any two of the following clinical symptoms of illness: ataxia, hunched posture, irregular respiration rate, ulceration of exposed tumor, or palpable tumor diameter exceeding 1.5 cm.

### Statistics

All statistical analyses were performed in GraphPad Prism, version 6.03. Statistical analyses of all in vitro cell culture experimentation, including flow cytometry analysis of cytokine production, viability, proliferation, and surface phenotype, kinetics of cell expansion, long term cytotoxicity, and chromium release assay by two-way ANOVA with donor-matching and Tukey’s post-test for multiple comparisons. Correlation of function with antigen density was performed by one-way ANOVA with post-test for linear trend. Analyses of in vivo bioluminescent imaging of tumor were performed using two-way ANOVA with repeated measures and Sidak’s post-test for multiple comparisons. Statistical analysis of animal survival data was performed by log-rank (Mantel-Cox) test. Significance of findings defined as follows: * p<0.05, ** p<0.01, *** p<0.001, **** p <0.0001.

### Example 2 - Numeric Expansion of T Cells by Artificial Antigen Presenting Cells Loaded with Anti-CD3

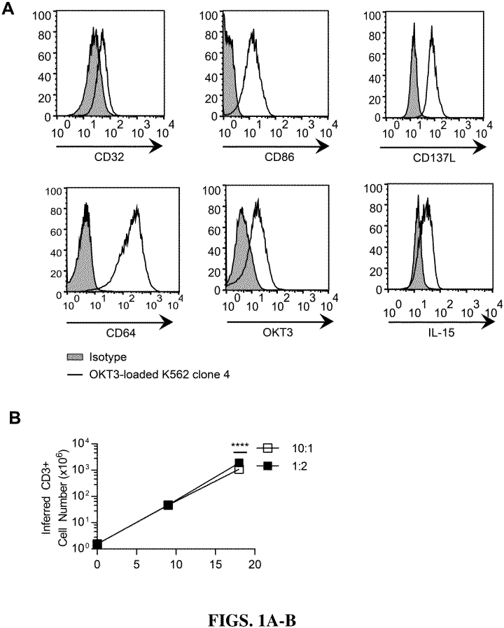

Antigen-dependent stimulation through stable CAR expression achieved by DNA integration can be used to numerically expand CAR⁺ T cells to clinically feasible numbers. The transient nature of CAR expression via RNA transfer requires numeric expansion of T cells to clinically feasible numbers to be achieved prior to RNA transfer of CAR. To determine the ability of aAPC to numerically expand T cells independent of antigen, anti-CD3 (OKT3) was loaded onto K562 via stable expression of the high affinity Fc receptor CD64 (FIG. 1A). K562 also expressed CD86, 41BB-L, and a membrane bound IL-15 for additional T-cell costimulation. To determine the impact of aAPC density in co-culture to stimulate T cell expansion, peripheral blood mononuclear cells (PBMC) derived from healthy human donors were co-cultured with γ-irradiated aAPC at low density, 10 T cells to 1 aAPC (10:1), or high density, 1 T cell to 2 aAPC (1:2), in the presence of IL-2. T cells were restimulated with aAPC after 9 days. Following two cycles of aAPC addition, T cells numerically expanded when stimulated 10:1 and 1:2 with aAPC; however, T cells with higher density of aAPC (1:2) achieved statistically superior numerical expansion (10:1 = 1083 ± 420 fold expansion, 1:2 = 1891 ± 376 fold expansion, mean ± S.D., n=6) (p<0.0001) (FIG. 1B).

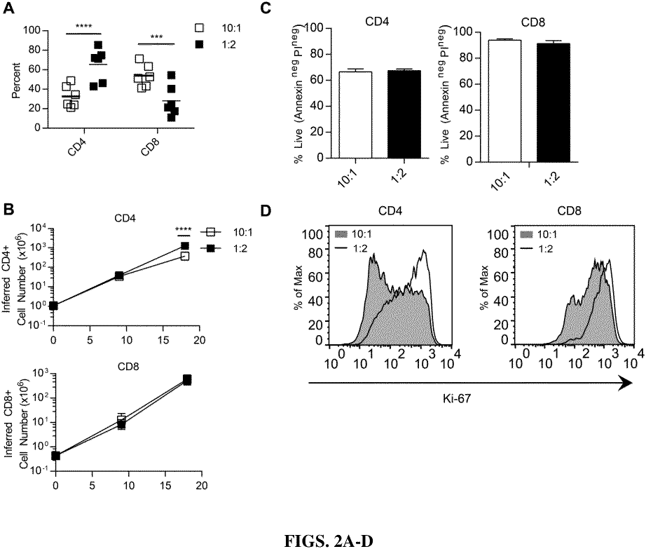

T cells expanded with lower density of aAPC contained a higher proportion of CD8⁺ T cells than T cells expanded with more aAPC (10:1 = 53.9 ± 11.6% CD8, 1:2 = 28.1 ± 16.2% CD8, mean ± S.D., n=6) (p<0.001) (FIG. 2A). CD8⁺ T cells demonstrated similar fold expansion in T cells when stimulated with either ratio of aAPC, however, CD4⁺ T cells demonstrated inferior fold expansion when stimulated with fewer aAPC (10:1 = 369 ± 227 CD4⁺ fold expansion, 1:2 = 1267 ± 447 CD4⁺ fold expansion, mean ± S.D., n=6) (p<0.0001) (FIG. 2B). To determine if reduced fold expansion was due to increased CD4⁺ T cell death in cultures with fewer aAPC, CD4⁺ and CD8⁺ T cells were stained with annexin V and propidium iodide (PI) and analyzed by flow cytometry to determine cell viability. There was no difference in the proportion of viable cells in CD4⁺ or CD8⁺ T cells when stimulated with low or high density aAPC (FIG. 2C). To determine if reduced fold expansion of CD4⁺ T cells was due to decreased rate of proliferation, T cells were stained 9 days following stimulation with aAPC for intracellular Ki-67 expression and analyzed by flow cytometry. CD8⁺ T cells demonstrated similar proliferation when stimulated with either low or high density of aAPC, however CD4⁺ T cells demonstrated reduced proliferation when stimulated with low density aAPC than high density aAPC (FIG. 2D). These data indicate that stimulating T cells with low density of aAPC results in less total T cells expansion than T cells stimulated with high density of aAPC, characterized by increased proportion of CD8⁺ T cells due to reduced proliferation of CD4⁺ T cells in response to low density of aAPC.

### Example 3 - T Cells Expanded With Lower Density aAPC Demonstrate a More Memory-like Phenotype Than T cells Expanded With Higher Density aAPC

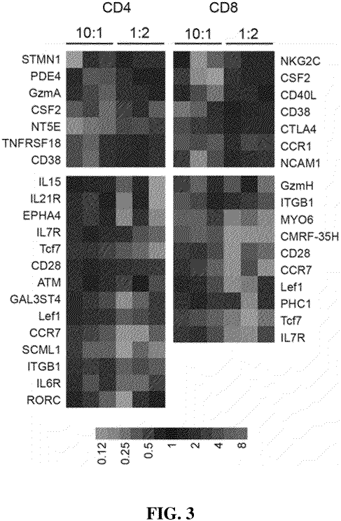

To determine if expansion with low density or high density aAPC impacted T-cell phenotype, expression of a panel of mRNA transcripts (Lymphocyte-specific CodeSet) was analyzed by multiplex digital profiling using nCounter analysis (Nanostring Technologies, Seattle, WA). Significant differential gene expression was determined by a p<0.01 and fold change greater than 1.5 in sorted CD4⁺ or CD8⁺ T cells expanded with low density (10:1 T cell:aAPC) or high density (1:2 T cell:aAPC) aAPC. CD4⁺ and CD8⁺ T cells expanded with high density aAPC demonstrated increased expression of genes associated with T-cell activation, such as CD38 and granzyme A in CD4⁺ T cells and CD38 and NCAM-1 in CD8⁺ T cells (FIG. 3). In contrast, CD4⁺ and CD8⁺ T cells expanded with low density aAPC showed increased expression of genes associated with central memory or naive T cells, including Wnt signaling pathway transcription factors Lef1 and Tcf7, CCR7, CD28, and IL7Rα (Gattinoni et al., 2009; Gattinoni et al., 2012).

To further evaluate differential phenotype of T cells expanded with low or high density aAPC, T cells were analyzed for phenotypic markers by flow cytometry and evaluated subsets by coexpression of CCR7 and CD45RA where CCR7⁺CD45RA⁺ indicates naïve phenotype, CCR7⁺CD45RA^{neg} indicates central memory phenotype, CCR7^{neg}CD45RA^{neg} indicates effector memory, and CCR7^{neg}CD45RA⁺ indicates a CD45RA⁺ effector memory phenotype (Geginat et al., 2003). CD4⁺ T cells expanded with low density aAPC contained significantly fewer T cells with effector memory phenotype (10:1=61.9 ± 9.1%, 1:2= 92.1 ± 3.9%, mean ± S.D., n=3) (p<0.05), but more central memory phenotype (10:1=36.5 ± 9.4%, 1:2=13.6 ± 2.4%, mean ± S.D., n=3) (p<0.05) T cells (FIG. 4A). Similarly, CD8⁺ T cells expanded with low density aAPC contained significantly fewer T cells with effector memory phenotype (10:1=66.1 ±12.5%, 1:2=89.1 ± 1.7%, mean ± S.D., n=3) (p<0.05), but more central memory phenotype (10:1=32.3 ± 11.7%, 1:2=6.5 ± 2.8%, mean ± S.D., n=3) (p<0.05). Significantly fewer CD4⁺ T cells stimulated with low density aAPC produce granzyme B (p<0.001) and fewer CD8⁺ T cells stimulated with low density aAPC produce granzyme B (p<0.05) or perforin (p<0.001) (FIG. 4B). When stimulated with PMA/Ionomycin, CD4⁺ T cells expanded with low and high density aAPC demonstrated equivalent production of IFN-γ, TNF-α, and IL-2, but CD8⁺ T cells stimulated with low density aAPC demonstrated significantly less production of IFN-γ (p<0.001) and TNF-α (p<0.05), but more production of IL-2 (p<0.05) (FIG. 4C). Collectively, these data suggest that T cells expanded with low density aAPC contain an increased proportion of T cells with central memory phenotype, reduced production of effector molecules granzyme B and perforin, and reduced production of effector cytokines IFN-γ and TNF-α compared to T cells expanded with higher density aAPC.

### Example 4 - Numeric Expansion of T Cells Results in Minimal Change in TCRαβ Diversity

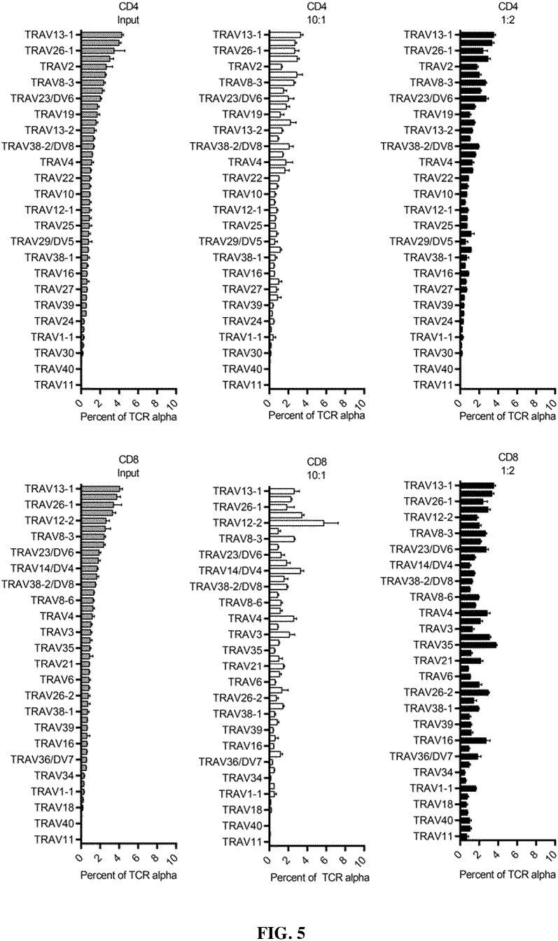

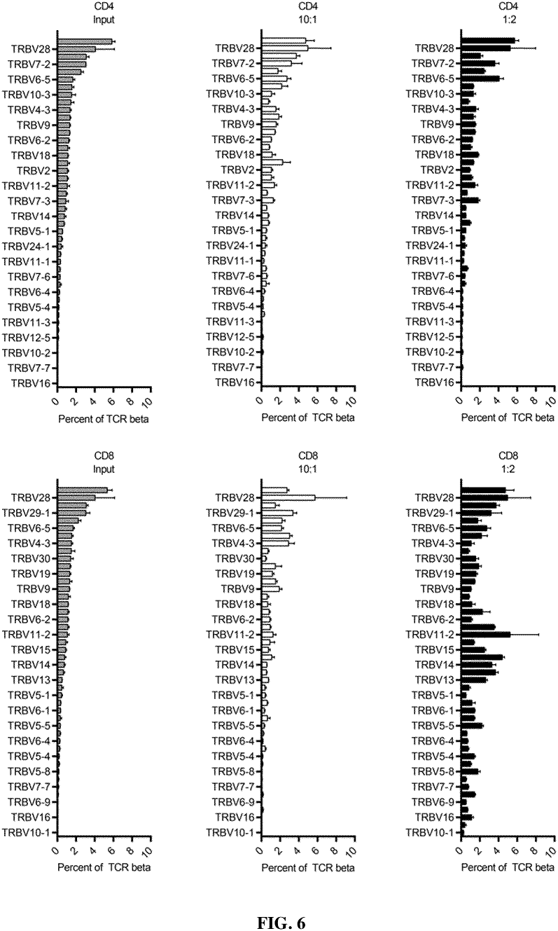

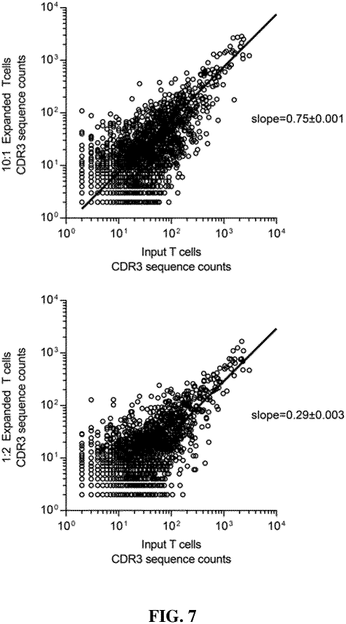

TCRα and TCRβ diversity was profiled prior to and following expansion with low and high density aAPC by multiplex digital profiling using nCounter analysis (Nanostring Technologies, Seattle, WA) and calculated the relative abundance of each TCRα and TCRβ chain as a percentage of total T-cell population. Following ex vivo expansion with low and high density aAPC, CD4⁺ and CD8⁺ T cells expressed diverse TCRα and TCRβ alleles, indicating that the resulting population maintained oligoclonal TCRα and TCRβ repertoire (FIG. 5 and FIG. 6). High throughput sequencing of CDR3 regions using the ImmunoSEQ platform (Adaptive TCR Technologies, Seattle, WA) in the TCRβ chain in T cells prior to and following expansion with low and high density of aAPC was performed to determine if ex vivo expansion resulted in change in clonal composition of T cells. Relative counts of individual CDR3 sequence prior to and following expansion were plotted and fitted with a linear regression. If the number of CDR3 sequences prior to and following expansion were identical, the slope of the linear regression would be expected to be 1.0. In T cells expanded with low density aAPC, the slope of the linear regression was 0.75 ± 0.001, while in T cells expanded with high density aAPC the slope of the linear regression was 0.29 ± 0.003 (FIG. 7). This indicates that T-cell populations expanded with low density aAPC maintain more CDR3 sequences from the input T-cell population than T cells expanded with high density aAPC. In sum, ex vivo expansion of T cells results in oligoclonal T-cell population when expanded with low and high density aAPC, but T cells expanded with low density aAPC may demonstrate less clonal loss following expansion.

### Example 5 - RNA Transfer to T Cells Numerically Expanded With aAPC

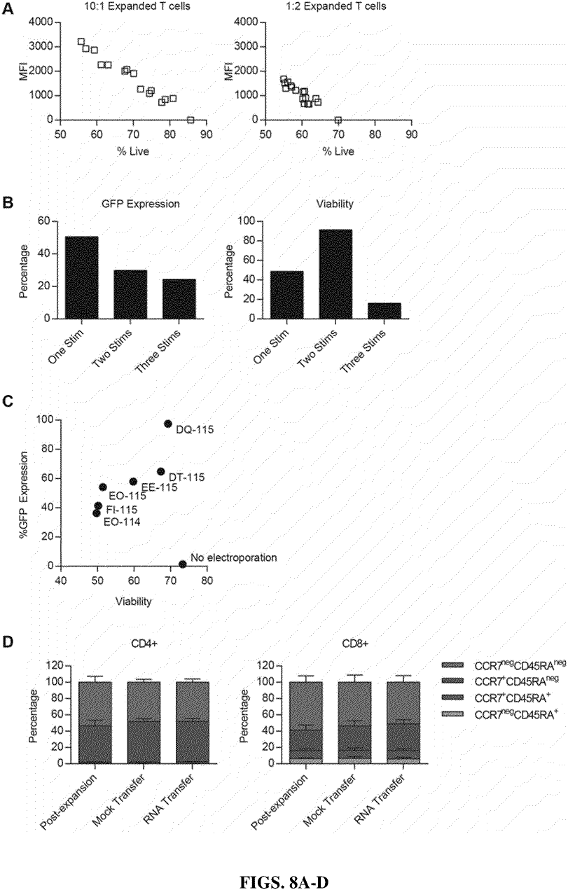

To determine the ability of T cells stimulated with low and high density aAPC to accept RNA by electro-transfer, in vitro transcribed RNA encoding green fluorescent protein (GFP) was electro-transferred using the Amaxa Nucleofector 4D transfection system (Lonza, Cologne, Germany) using a variety of electroporation programs, including program EO-115, the manufacturer’s recommended program for stimulated T cells 4 days following stimulation with aAPC. Plotting the mean fluorescent intensity (MFI) of GFP versus the viability of T cells determined by PI staining revealed an inverse correlation between GFP expression and T-cell viability following RNA transfer. Compared to T cells stimulated with low density aAPC, T cells stimulated with high density aAPC demonstrated both reduced expression of GFP by RNA transfer and reduced viability in response to every electroporation program tested (FIG. 8A). As a result, T cells stimulated with low density aAPC (10 T cells to 1 aAPC) were used in all further experiments. Because T-cell numeric expansion prior to RNA transfer is desirable to achieve clinically relevant T-cell numbers for infusion, the capacity of T cells undergoing multiple rounds of stimulation by recursive addition of aAPC every 9 days to accept RNA transcripts by electro-transfer was evaluated. In each successive round of stimulation, expression of GFP following RNA electro-transfer decreased (FIG. 8B, left panel). However, following two rounds of stimulation, T cells demonstrated improved viability after electro-transfer compared to T cells undergoing a single round of stimulation or three rounds of stimulation (FIG. 8B, right panel). Therefore, a stimulation protocol of two rounds of stimulation with 10 T cells to 1 aAPC was selected for further optimization of RNA transcript transfer. Because RNA is less toxic to cells and transferred more readily into many cell types than DNA (165), it was reasoned that RNA transfer efficiency could be improved without compromising T-cell viability by decreasing the strength of the manufacturer recommended electroporation program for stimulated T cells, EO-115. By plotting the percentage of cells expressing GFP versus viability determined by PI staining, a program was identified that resulted in ~100% GFP expression 24 hours following electroporation and similar T-cell viability as T cells that were not electroporated, program DQ-115 (FIG. 8C). T-cell phenotype was assessed following electroporation with the optimized protocol to determine if electro-transfer of RNA would alter T-cell phenotype. No changes in T-cell phenotype were detected following electroporation with or without RNA transcripts (FIG. 8D). Thus, a platform was developed for RNA transfer to T cells that following numeric expansion via co-culture with aAPC that resulted in high expression of RNA transcript without compromising T-cell viability.

### Example 6 - CAR Expression and Phenotype T Cells Modified by DNA or RNA Transfer

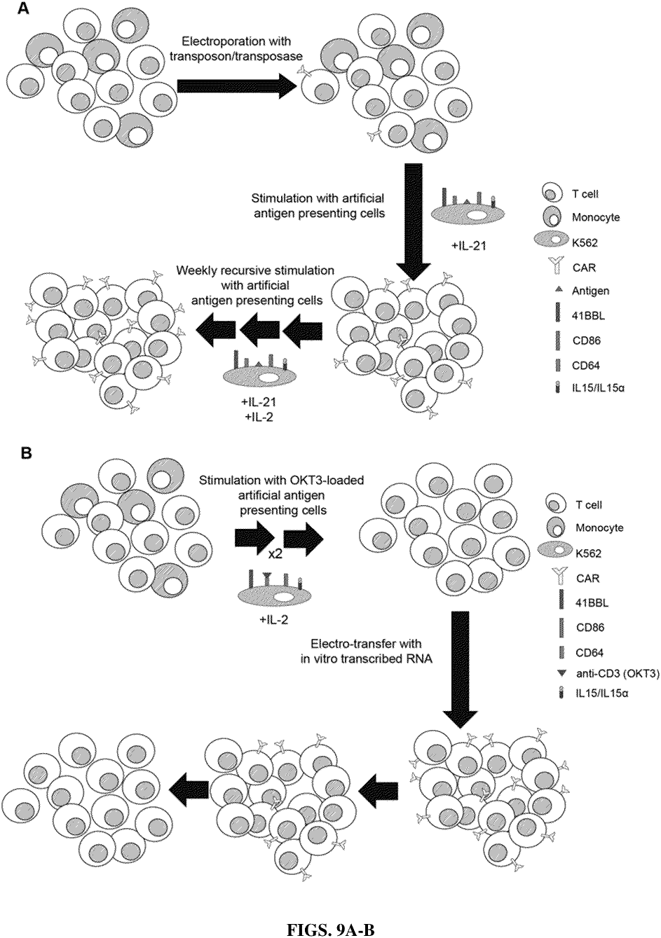

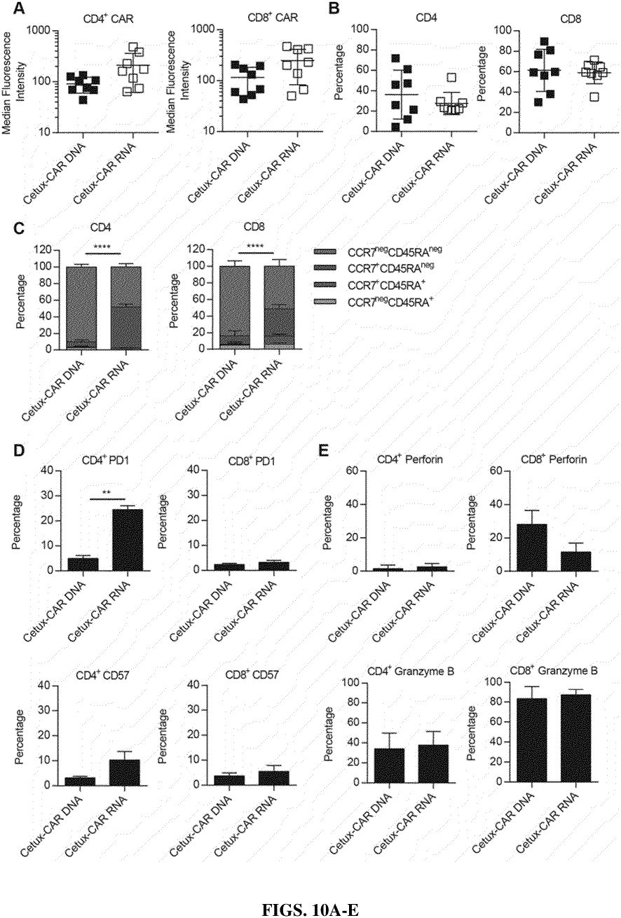

To compare expression of CAR and function of CAR⁺ T cells manufactured by RNA and DNA modification, an EGFR-specific CAR was developed from the scFv of cetuximab, a clinically available anti-EGFR monoclonal antibody. The scFv of cetuximab was fused to an IgG4 hinge region, CD28 transmembrane and cytoplasmic domains, and CD3-ζ cytoplasmic domain to form a second generation CAR, termed Cetux-CAR, and expressed in a Sleeping Beauty transposon for permanent DNA integration as well as under a T7 promoter in the pGEM/A64 vector for in vitro transcription of RNA transcripts. RNA-modification of T cells was achieved by electro-transferring in vitro transcribed Cetux-CAR into T cells stimulated twice with OKT3-loaded K562 aAPC, four days following the second stimulation (FIG. 9A). CAR expression was evaluated 24 hours following electro-transfer. For stable DNA integration, Cetux-CAR expressed in SB transposon was electroporated into human primary T cells with the SB11 transposase, a cut-and-paste enzyme, which excises the CAR from the transposon and inserts into the host T-cell genome at inverted TA repeats. Recursive stimulation with γ-irradiated EGFR⁺ K562 aAPC results in selective expansion of CAR-expressing T cells over time, and T cells were evaluated for CAR expression following 28 days consisting of 5 cycles of recursive aAPC addition, every 7 days (FIG. 9B). Expression of Cetux-CAR by RNA-modification and DNA-modification in CD4⁺ and CD8⁺ as determined by flow cytometry for the IgG4 hinge region of CAR was not statistically different (p>0.05), however, RNA-modification resulted in greater variation in expression intensity (FIG. 10A). Of Cetux-CAR-expressing T cells, the proportion of CD4⁺ and CD8⁺ T cells was not statistically different between T cells modified with RNA or DNA, however, there was greater variability in the proportion of CD4⁺ and CD8⁺ T cells present in DNA-modified than RNA-modified CAR⁺ T cells (FIG. 10B).

To compare the phenotype of T-cell populations expressing Cetux-CAR by RNA-modification or DNA-modification, phenotypic markers were analyzed by flow cytometry. CD4⁺ RNA-modified CAR⁺ T cells had significantly more T cells with central memory phenotype than CD4⁺ DNA-modified CAR⁺ T cells (CCR7⁺CD45RA^{neg}) (DNA-modified=6.6 ± 1.9%, RNA-modified=49.6 ± 3.0%, mean ± S.D., n=3) (p<0.0001), but significantly fewer T cells with effector memory phenotype (CCR7^{neg}CD45RA^{neg}) (DNA-modified=89.8 ± 2.6%, RNA- modified=48.1 ± 3.3%, mean ± S.D., n=3) (p<0.0001) (FIG. 10C). Similarly, CD8⁺ RNA-modified CAR⁺ T cells had significantly more T cells with central memory phenotype than CD8⁺ DNA-modified CAR⁺ T cells (DNA-modified=10.4 ± 4.9%, RNA-modified=32.8 ± 4.2%, mean ± S.D., n=3) (p<0.001), but significantly fewer T cells with effector memory phenotype (DNA-modified=83.5 ± 5.4%, RNA-modified=51.1 ± 6.6%, mean ± S.D., n=3) (p>0.0001). CD4⁺ Cetux-CAR⁺ T cells modified by RNA also demonstrated significantly higher expression of the inhibitory receptor programmed death receptor 1 (PD-1) than CD4⁺ Cetux-CAR⁺ T cells, (p<0.01), but similar, low expression of CD57, a marker of T-cell senescence (FIG. 10D). CD8⁺ Cetux-CAR⁺ T cells expressed low levels of PD-1 and CD57 and there was no appreciable difference RNA-modified and DNA-modified CAR⁺ T cells. Finally, expression of the cytotoxic molecules perforin and granzyme B, was similar in CD4⁺ and CD8⁺ T cells modified by DNA or RNA transfer of Cetux-CAR (FIG. 10E). In sum, RNA-modification and DNA-modification of CAR⁺ T cells resulted in similar expression levels of CAR, though RNA transfer resulted in increased variability of the intensity of CAR expression. RNA-modified T cells expressed more central memory phenotype CD4⁺ and CD8⁺ T cells, less effector memory phenotype CD4⁺ and CD8⁺ T cells, and had higher expression of inhibitory receptor PD-1 on CD4⁺ CAR⁺ T cells than DNA-modified T cells.

### Example 7 - DNA-modified CAR⁺ T cells Produce More Cytokine and Display Slightly More Cytotoxicity Than RNA-modified CAR⁺ T cells

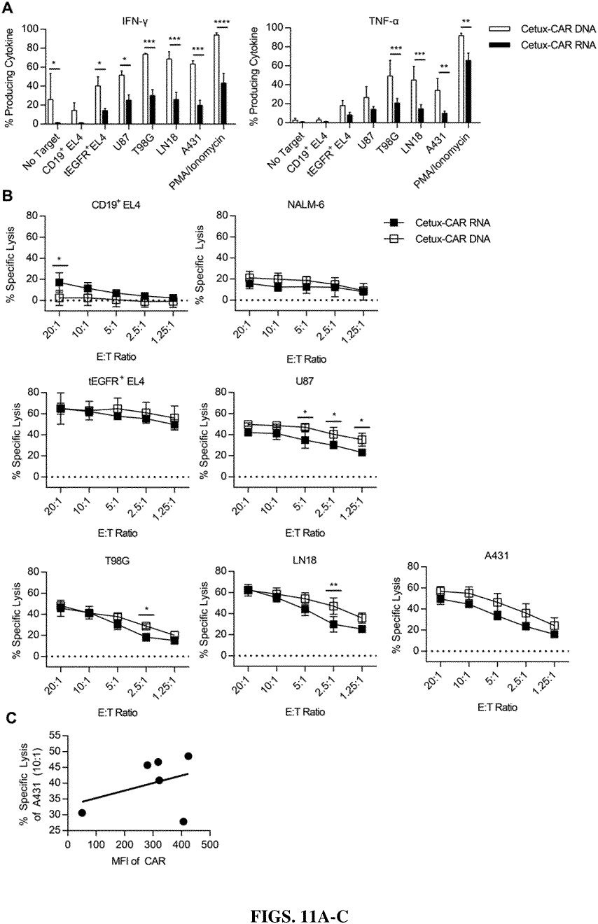

Cytokine production of RNA-modified or DNA-modified CAR⁺ T cells was evaluated in response to a mouse T cell lymphoma cell line EL4 modified to express truncated EGFR, tEGFR⁺ EL4, or irrelevant antigen, CD19, and EGFR⁺ cell lines, including human glioblastoma cell lines U87, T98G, LN18 and human epidermoid carcinoma cell line A431. Fewer CD8⁺ CAR⁺ T cells modified by RNA transfer produced IFN-γ in response to all EGFR-expressing cell lines (FIG. 11A, left panel). Because fewer RNA-modified T cells produced IFN-γ in response to antigen-independent stimulation with PMA/Ionomycin, it is not likely that reduced IFN-γ production is due to reduced sensitivity of CAR to antigen, but rather reduced capacity of T cells expressing CAR by RNA-modification to produce cytokine. It was noted that DNA-modified CAR⁺ T cells also demonstrated higher background production of IFN-γ in the absence of T cell stimulation. Similarly, fewer RNA-modified CD8⁺ CAR⁺ T cells produced TNF-α in response to EGFR-specific stimulation from T98G, LN18, A431 and antigen-independent stimulation from PMA/Ionomycin than DNA-modified CD8⁺ CAR⁺ T cells (FIG. 11A, right panel).

Because RNA-modified CAR⁺ T cells demonstrated reduced capacity to produce cytokine relative to DNA-modified CAR⁺ T cells, cytotoxicity of RNA-modified and DNA-modified T cells was compared to determine the cytotoxic potential of RNA-modified CAR⁺ T cells relative to DNA-modified CAR⁺ T cells. In response to CD19⁺ EL4 cells, RNA-modified and DNA-modified CAR⁺ T cells had low levels of background killing, although at high effector to target ratio (E:T = 20:1), RNA-modified CAR⁺ T cells demonstrated significantly more background lysis than DNA-modified CAR⁺ T cells (p<0.05) (FIG. 11B) . Similarly, RNA-modified and DNA-modified CAR⁺ T cells demonstrated low and equivalent levels of background lysis against B-cell lymphoma cell line, NALM-6. In response to tEGFR⁺ EL4 and A431, there was no appreciable difference in cytotoxicity mediated by RNA-modified or DNA-modified CAR⁺ T cells. In response to the three glioma cell lines U87, T98G, and LN18, DNA-modified CAR⁺ T cells demonstrated slightly increased cytotoxicity over RNA-modified CAR⁺ T cells only detected at low E:T ratios. Because RNA-modified T cells have more variability in CAR expression than DNA-modified T cells from donor to donor, the impact of CAR expression, as determined by median fluorescence intensity of CAR expression, on specific lysis of A431 was evaluated. Median fluorescence intensity of CAR expression was plotted versus specific lysis of A431, and a linear regression of the relationship yielded a slope not significantly different than zero, and therefore, showed no significant trend detected between CAR expression and specific lysis (slope=0.0237±0.030, p=0.4798) (FIG. 11C). In sum, these findings suggest that DNA-modified CAR⁺ T cells have significantly increased production of effector cytokines IFN-γ and TNF-α relative to RNA-modified CAR⁺ T cells, may demonstrate slightly more cytotoxicity when present at low E:T ratios, and that the variability of CAR expression in RNA-modified CAR⁺ T cells does not significantly impact specific lysis of targets.

### Example 8 - Transient Expression of Cetux-CAR by RNA Modification of T Cells

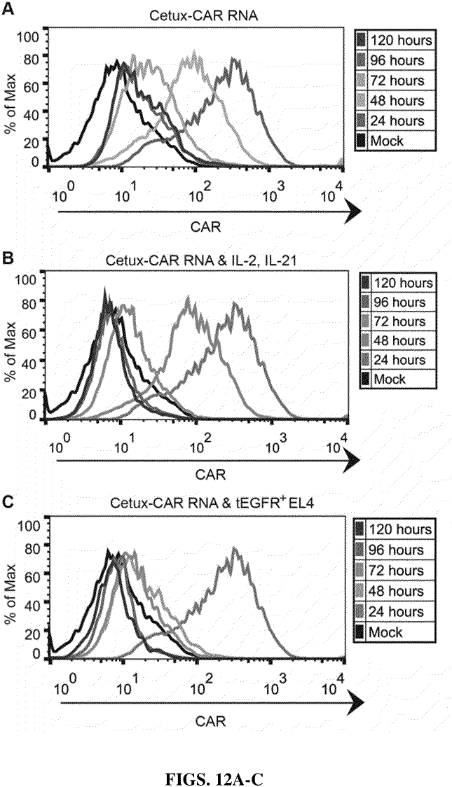

To determine the stability of CAR expression by RNA transfer, T cells were modified to express CAR by RNA transfer, and CAR expression was measured over time by flow cytometry. Following RNA transfer, expression of Cetux-CAR on T cells decreased over time, and 96 hours following electro-transfer, CAR was expressed at low levels (FIG. 12A). Because RNA transcripts are divided between daughter cells during T cell proliferation, stimulation of T cell proliferation should accelerate the loss of CAR expressed by RNA-modification. To determine the effect of cytokine stimulation on CAR expression level, exogenous IL-2 and IL-21 were added to RNA-modified CAR⁺ T cell culture 24 hours after RNA transfer and CAR expression was monitored by flow cytometry. Stimulation of CAR⁺ T cells with IL-1 and IL-21 accelerated the loss of CAR expression (FIG. 12B). Following 72 hours, CAR expression was low on RNA-modified T cells, and 96 hours after transfer, T cells were no longer expressed CAR at a detectable level. Stimulation of RNA-modified CAR⁺ T cells with tEGFR⁺ EL4 24 hours after RNA transfer accelerated the loss of CAR expression even further (FIG. 12C). While CAR was detected at high level in RNA-modified CAR⁺ T cells prior to addition of tEGFR⁺ EL4, 24 hours after tEGFR⁺ EL4 addition (48 hours following RNA transfer), CAR expression was low. Collectively, these data indicate the CAR expression by RNA transfer is transient, detectable at low levels up to 120 hours after RNA transfer, however, stimulation of T cells through cytokine or recognition of antigen accelerated the loss of CAR expression.

### Example 9 - Transient Expression of Cetux-CAR by RNA Modification Reduces Cytokine Production and Cytotoxicity to EGFR-expressing Cells

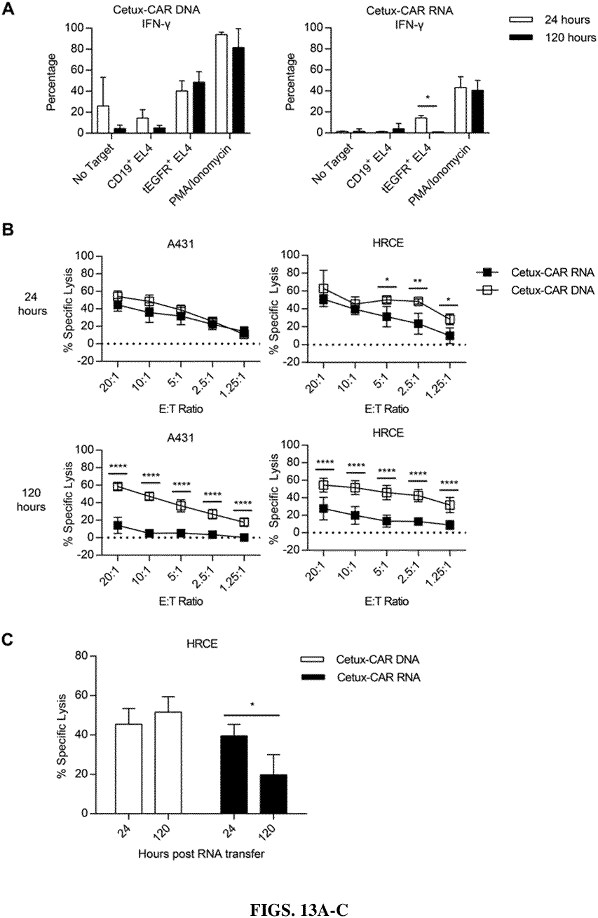

Activity of T cells modified to express Cetux-CAR by RNA transfer was measured 24 and 120 hours after RNA transfer to determine the effect of loss of CAR expression on activity of T cells in response to EGFR-expressing cells. While RNA-modified T cells demonstrated equivalent production of IFN-γ by PMA/Ionomycin stimulation when assessed at 24 hours and 120 hours after RNA transfer, production of IFN-γ in response to tEGFR⁺ EL4 by T cells 24 hours after RNA transfer was abrogated 120 hours after RNA transfer (24 hrs=14.2 ± 2.5%, 120 hrs=1.1 ± 0.03%, mean ± S.D., n=3) (p=0.012) (FIG. 13A). In contrast, DNA-modified CAR⁺ T cells demonstrated equivalent production of IFN-γ in response to tEGFR⁺ EL4 at both time points assessed (24 hrs=40.3 ± 9.6%, 120 hrs=48.6 ± 10.0%, mean ± S.D., n=3) (p=0.490). Similarly, specific cytotoxicity was measured against epidermoid carcinoma cell line A431 and human normal kidney epithelial cells (HRCE), which express EGFR. RNA-modified and DNA-modified CAR⁺ T cells demonstrated equivalent specific lysis of A431, and similar cytotoxicity against HRCE, statistically equivalent at higher effector to target ratios (20:1 and 10:1, p>0.05) (FIG. 13B). Similar to observations with other cell lines, DNA-modified CAR⁺ T cells mediated slightly higher specific lysis of HRCE than RNA-modified CAR⁺ T cells at lower E:T ratios (5:1, p<0.05; 2.5:1, p<0.01, 1.25:1, p<0.05). However, 120 hours after RNA transfer, when CAR expression of RNA-modified T cells is abrogated, DNA-modified T cells mediated significantly higher specific lysis in response to A431 and HRCE at every E:T ratio evaluated (A431, all E:T ratios, p<0.0001; HRCE, all E:T ratios, p<0.0001). While DNA-modified T cells demonstrated no change in specific lysis of HRCE at each time point (10:1 E:T ratio, 24 hrs=45.5 ± 8.0%, 120 hrs=51.6 ± 7.8%, p>0.05, n=3), RNA-modified T cells significantly reduced specific lysis of HRCE by 120 hours after RNA transfer (10:1 E:T ratio, 24 hrs=39.5 ± 5.9%, 120 hrs=19.8 ± 10.2%, mean ± S.D., n=3) (FIG. 13C). These data indicate that activity of RNA-modified, but not DNA-modified, T cells in response to EGFR-expressing targets is reduced by loss of CAR expression.

### Example 10 - Cetux-CAR⁺ and Nimo-CAR⁺ T cells are Phenotypically Similar

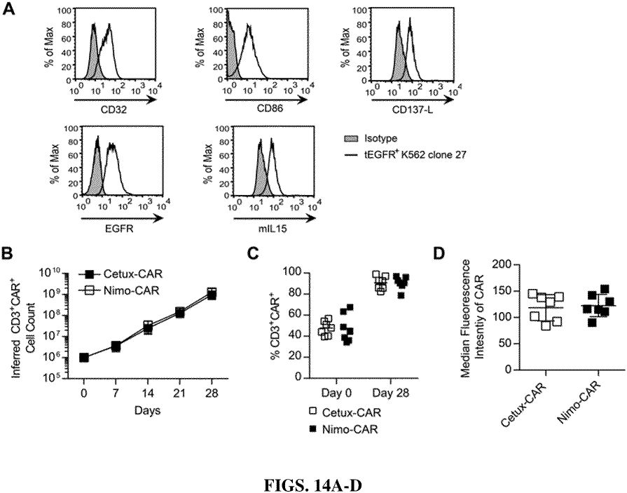

A second generation CAR derived from nimotuzumab, designated Nimo-CAR, was generated in a Sleeping Beauty transposon by fusing the scFv of nimotuzumab with an IgG4 hinge region, CD28 transmembrane domain and CD28 and CD3ζ intracellular domains, an identical configuration to Cetux-CAR. Cetux-CAR and Nimo-CAR were expressed in primary human T cells by electroporation of each transposon with SB11 transposase into peripheral blood mononuclear cells (PBMC). T cells with stable integration of Cetux-CAR or Nimo-CAR were selectively propagated by weekly recursive stimulation with γ-irradiated tEGFR⁺ K562 artificial antigen presenting cells (aAPC) (FIG. 14A). Both CARs mediated ~1000-fold expansion of CAR⁺ T cells over 28 days of co-culture with aAPC, yielding T cells which almost all expressed CAR (Cetux-CAR=90.8±6.2%, Nimo-CAR=90.6±6.1%; mean±SD, n=7) (FIGS. 14B and 14C). Proportion of Cetux-CAR and Nimo-CAR⁺ T cells expressing CAR was statistically similar following 28 days of numeric expansion (p=0.92, student’s two-tailed t-test). Density of CAR expression, represented by median fluorescence intensity, was measured by flow cytometry and was statistically similar between Cetux-CAR⁺ and Nimo-CAR⁺ T cell populations (Cetux-CAR=118.5±25.0 A.U., Nimo-CAR=112.6±21.2 A.U.; mean±SD, n=7) (p=0.74) (FIG. 14D).

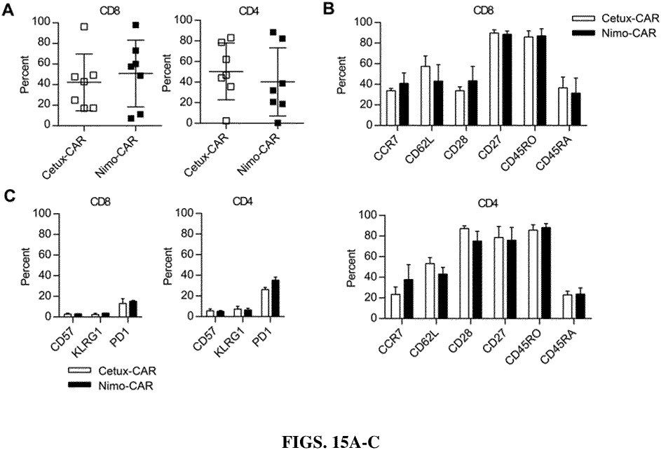

In order to determine the impact of CAR scFv on T-cell function, electroporation and propagation of Cetux-CAR⁺ and Nimo-CAR⁺ T cells were established to result in phenotypically similar T-cell populations. Each donor yielded variable ratios of CD4⁺ and CD8⁺ T cells (Table 1), however, there was no statistical difference in the CD4/CD8 ratio between Cetux-CAR⁺ and Nimo-CAR⁺ T cells (p=0.44, student’s two-tailed t-test) (FIG. 15A). Expression of differentiation markers CD45RO, CD45RA, CD28, CD27, CCR7 and CD62L were not statistically significant (p>0.05), and indicate a heterogeneous T-cell population (FIG. 18B). Likewise, markers for senescence CD57 and KLRG1 and the inhibitory receptor programmed death receptor 1 (PD-1) were found to be low and not statistically different between Cetux-CAR⁺ and Nimo-CAR⁺ T-cell populations (p>0.05) (FIG. 15C). In aggregate, these findings indicate that Cetux-CAR⁺ and Nimo-CAR⁺ T cells have no detectable phenotypic differences, including CAR expression, after electroporation and propagation, enabling direct comparison.

TABLE 1

Ratio of CD4 and CD8 in Cetux-CAR⁺ and Nimo CAR⁺ T cells

| Donor | Cetux-CAR %CD4 | Cetux-CAR %CD8 | Cetux-CAR Ratio (CD4/CD8) | Nimo-CAR %CD4 | Nimo-CAR %CD8 | Nimo-CAR Ratio (CD4/CD8) |
|---|---|---|---|---|---|---|
| 1 | 46.7 | 42.9 | 1.09 | 18.8 | 73.1 | 0.26 |
| 2 | 83.0 | 17.3 | 4.80 | 88.4 | 7.17 | 12.3 |
| 3 | 2.5 | 96.2 | 0.03 | 0.4 | 97.9 | 0.01 |
| 4 | 62.0 | 24.9 | 2.49 | 38.7 | 48. | 0.79 |
| 5 | 35.5 | 47.6 | 0.75 | 20.8 | 57.6 | 0.36 |
| 6 | 78.5 | 17.1 | 4.59 | 82.3 | 11.3 | 7.29 |
| 7 | 44.0 | 49.2 | 0.89 | 31.9 | 60.1 | 0.53 |

Expression of CD4 and CD8 in Cetux-CAR⁺ and Nimo-CAR⁺ T cells after 28 days of expansion was determined by flow cytometry. Data from 7 independent donors.

### Example 11 - Cetux-CAR⁺ and Nimo-CAR⁺ T cells Have Equivalent Capacity for CAR-Dependent T-cell Activation

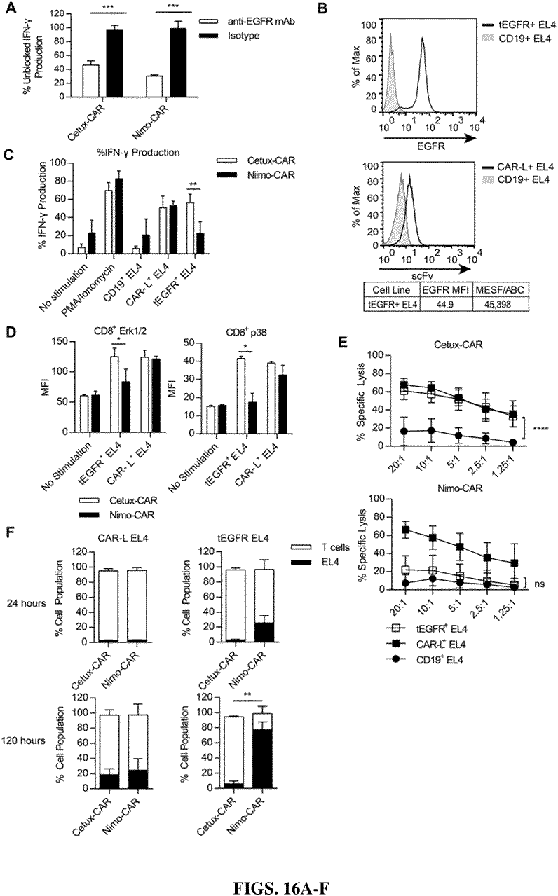

To verify Cetux-CAR and Nimo-CAR were functional in response to stimulation with EGFR, CAR⁺ T cells were incubated with the A431 epidermoid carcinoma cell line, which is reported to express high levels of EGFR, about 1x10⁶ molecules of EGFR/cell (Garrido et al., 2011). Cetux- and Nimo- CAR⁺ T cells produced IFN-γ during co-culture with A431, which was reduced in the presence of anti-EGFR monoclonal antibody that blocks binding to EGFR (FIG. 16A). To verify that Cetux-CAR and Nimo-CAR are equivalently capable of activating T cells, targets were generated that could be recognized by both CARs independent of the scFv domain. This was accomplished by expressing the scFv region of an activating antibody specific for the IgG4 region of CAR (CAR-L) on immortalized mouse T cell line EL4 (Rushworth et al., 2014). Activation of T cells by CAR-L⁺ EL4 was compared to activation by an EL4 cell line expressing tEGFR. Quantitative flow cytometry was performed to measure the density of tEGFR expressed on EL4. In this method, intensity of fluorescence from microspheres with a known antibody binding capacity labeled with fluorescent antibody is measured by flow cytometry and used to derive a standard curve, which defines a linear relationship between known antibody binding capacity and mean fluorescence intensity (MFI). The standard curve can then be used to derive the mean density of antigen expression from the mean fluorescence intensity of an unknown sample labeled with the same fluorescent antibody. tEGFR⁺ EL4 expressed tEGFR at a relatively low density, about 45,000 molecules/cell (FIG. 16B). Cetux-CAR⁺ and Nimo-CAR⁺ CD8⁺ T cells demonstrated statistically similar amounts of IFN-γ in response to CAR-L⁺ EL4s, indicating equivalent capacity for CAR-dependent activation (p>0.05) (FIG. 16C). While Cetux-CAR⁺ T cells produced IFN-γ in response to EGFR⁺, there was no appreciable IFN-γ production from Nimo-CAR⁺ T cells (FIG. 16C), which is consistent with the affinity of the scFv of CAR impacting T cell activation in response to low antigen density. In addition to measuring cytokine production, CD8⁺ T cells were analyzed for phosphorylation of molecules downstream of T-cell activation, Erk1/2 and p38. There was no statistical difference in phosphorylation of Erk1/2 (p>0.05) or p38 (p>0.05) between Cetux-CAR⁺ and Nimo-CAR⁺ T cells in response to CAR-L⁺ EL4 (FIG. 16D). While Cetux-CAR⁺ T cells exhibited phosphorylation of Erk1/2 and p38 in response to tEGFR⁺ EL4, Nimo-CAR⁺ T cells failed to appreciably phosphorylate either molecule. Similarly, Cetux-CAR and Nimo-CAR both demonstrated equivalent specific lysis against CAR-L⁺ EL4 (10:1 E:T ratio, Cetux-CAR=64.5±6.7%, Nimo-CAR=57.5±12.9%, mean±SD, n=4)(p>0.05). While Cetux-CAR⁺ T cells demonstrated significant specific lysis in response to tEGFR⁺ EL4 over non-specific targets CD19⁺EL4 (tEGFR⁺EL4=57.5±9.4%, tCD19⁺EL4=17.3±13.0, mean±SD, n=4) (p<0.0001), there was not significant lysis of tEGFR⁺ EL4 by Nimo-CAR⁺ T cells (tEGFR⁺EL4=21.2±16.9%, CD19⁺EL4=12.3±13.0, mean±SD, n=4) (p>0.05) (FIG. 16E). Endogenous, low-affinity T cell responses may require longer interaction with antigen to achieve effector function (Rosette et al., 2001), therefore, the ability of CAR⁺ T cells to control growth of t EGFR⁺ and CAR-L⁺ EL4 cells was evaluated by mixing T cells with EL4s at a ratio of 1:1 and evaluating proportion of T cells to EL4 cells over an extended co-culture. Cetux-CAR⁺ T cells and Nimo-CAR⁺ T cells controlled growth of CAR-L⁺ EL4s equivalently (p>0.05), as demonstrated by low proportion of CAR-L⁺ EL4 cells in co-culture after 5 days (FIG. 16F). Cetux-CAR⁺ T cells controlled growth of tEGFR⁺EL4, resulting in less than 10% of tEGFR⁺ EL4 in the co-culture after 5 days. Nimo-CAR⁺ T cells were less capable of controlling tEGFR⁺ EL4 cell growth, resulting in tEGFR⁺ EL4 accounting for 80% of the co-culture after 5 days, significantly more than co-culture with Cetux-CAR⁺T cells (p<0.01). Therefore, reduced response by Nimo-CAR⁺ T cells to low tEGFR density on tEGFR⁺ EL4 is not likely due to insufficient time for activation. In sum, these data demonstrate that Cetux-CAR⁺ and Nimo-CAR⁺ T cells have functional specificity for EGFR and can be equivalently activated by CAR-dependent, scFv-independent stimulation. Cetux-CAR⁺ T cells were capable of specific activation in response to low tEGFR density on tEGFR⁺ EL4; however, this density of EGFR expression was not sufficient for activation Nimo-CAR⁺ T cells to produce cytokine, phosphorylate downstream molecules Erk1/2 and p38, or initiate specific lysis.

### Example 12 - Activation and Functional Response of Nimo-CAR⁺ T cells is Impacted by Density of EGFR Expression on Target Cells

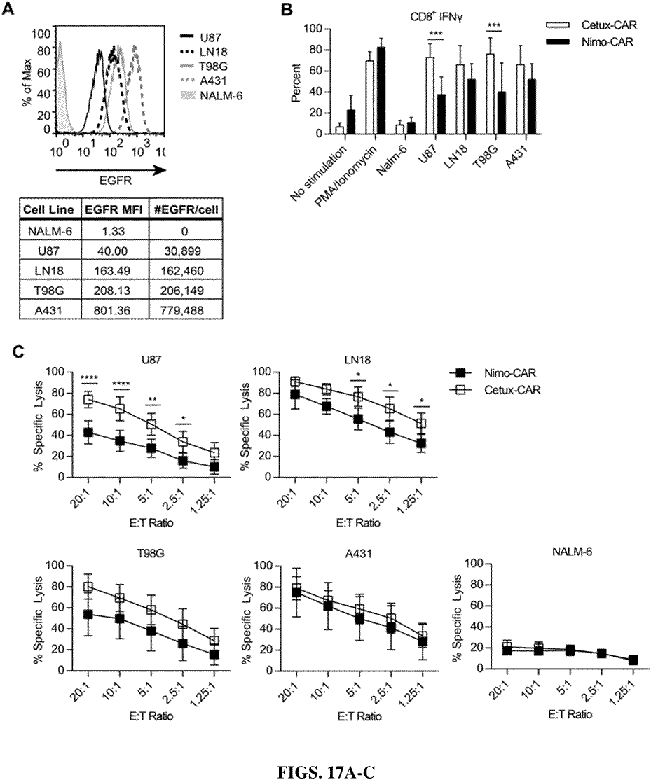

To investigate the impact of EGFR expression density on activation of Cetux-CAR⁺ and Nimo- CAR⁺ T cells, T-cell function was compared against cell lines with a range of EGFR expression density: NALM-6, U87, LN18, T98G, and A431. First, EGFR expression density was evaluated by quantitative flow cytometry (FIG. 17A). NALM-6, a B-cell leukemia cell line, expressed no EGFR. U87, a human glioblastoma cell line, expressed EGFR at low density (~30,000 molecule/cell). LN18 and T98G, both human glioblastoma cell lines, expressed EGFR at intermediate density (~160,000 and ~205,000 molecules/cell, respectively), and A431 was found to expression EGFR at high density (~780,000 molecules/cell), similar to previous reports (Garrido et al., 2011). Cetux-CAR⁺ and Nimo-CAR⁺ CD8⁺ T cells demonstrated statistically similar IFN-γ production in response to A431 with high EGFR density (p>0.05) and LN18 with intermediate EGFR density (p>0.05). However, Nimo-CAR⁺ T cells demonstrated reduced IFN-γ production in response to T98G with intermediate EGFR density (p<0.001) and U87 with low EGFR density (p<0.001) relative to Cetux-CAR⁺ T cells (FIG. 17B). Similarly, while Cetux-CAR⁺ and Nimo-CAR⁺ T cells demonstrated statistically equivalent lysis of A431 cells (5:1 E:T ratio, p>0.05) and T98G cells (5:1 E:T ratio, p>0.05), Nimo-CAR⁺ T cells demonstrated some reduced capacity for specific lysis of LN18 cells (5:1 E:T ratio, p<0.05) and reduced capacity for specific lysis of U87 cells (5:1 E:T ratio, p< 0.01) (FIG. 17C). These data support that activation of Nimo-CAR⁺ T cells is impacted by the density of EGFR expression. However, evaluating function against EGFR density in the context of different cellular backgrounds is not ideal since different cell lines may have different propensity for T-cell activation and susceptibility to T-cell mediated lysis.

### Example 13 - Activation of Function of Nimo-CAR⁺ T cells is Directly and Positively Correlated With EGFR Expression Density

To determine the impact of EGFR expression density on a syngeneic cellular background, a series of U87 cell lines expressing varying densities of EGFR was developed: unmodified, parental U87 (~30,000 molecules of EGFR/cell), U87low (130,000 molecules of EGFR/cell), U87med (340,000 molecules of EGFR/cell), and U87high (630,000 molecules of EGFR/cell) (FIG. 18A). To compare phosphorylation of Erk1/2 and p38 following scFv-dependent CAR stimulation, it was ensured that there was not a distinction in kinetics of phosphorylation between Nimo-CAR⁺ T cells and Cetux-CAR⁺ T cells following stimulation U87 and U87high. Both CD8⁺CAR⁺ T cells demonstrated peak phosphorylation of Erk1/2 and p38 45 minutes after interaction and phosphorylation began to decrease by 120 minutes after interaction (FIG. 18B). There was no appreciable distinction in phosphorylation kinetics between Cetux-CAR⁺ T cells and Nimo-CAR⁺ T cells and future experiments assessed phosphorylation of Erk1/2 and p38 45 minutes following interaction for all future experiments. Cetux-CAR⁺ CD8⁺ T cells phosphorylated Erk1/2 and p38 in response to all four U87 cell lines and showed no correlation with density of EGFR expression (one-way ANOVA with post-test for linear trend; Erk1/2, p=0.88; p38, p=0.09) (FIG. 18C). In contrast, phosphorylation of Erk1/2 and p38 by Nimo-CAR⁺ CD8⁺ T cells directly correlated with EGFR expression density (one-way ANOVA with post-test for linear trend, Erk1/2 p = 0.0030 and p38 p=0.0044). Itwas noted that Nimo-CAR⁺ T cells demonstrated significantly less phosphorylation pf Erk1/2 and p38 than Cetux-CAR⁺ T cells, even in response to high EGFR density on U87high (Erk1/2, p<0.0001; p38, p<0.01). Similarly, Cetux-CAR⁺ CD8⁺ T cells produced IFN-γ and TNF-α in response to U87, U87low, U87med and U87high, and production did not correlate with EGFR expression density (one-way ANOVA with post-test for linear trend; IFN-γ, p = 0.5703 and TNF-α, p=0.6189) (FIG. 18D). In contrast, Nimo-CAR⁺ CD8⁺ T cells produced IFN-γ and TNF-α in direct correlation with EGFR expression density (one-way ANOVA with post-test for linear trend; IFN-γ, p = 0.0124 and TNF-α, p=0.0006). Cetux-CAR⁺ CD8⁺ T cells produced significantly more cytokine than Nimo-CAR⁺ CD8⁺ T cells in response to stimulation with U87 (IFN-γ, p<0.0001; TNFα, p<0.01) or U87low (IFN-γ, p<0.001; TNFα, p<0.01), however, Cetux-CAR⁺ T cells and Nimo-CAR⁺ T cells demonstrated statistically similar cytokine production in response to stimulation with U87med (IFN-γ, p>0.05; TNFα, p>0.05) or U87high (IFN-γ, p>0.05; TNFα, p>0.05). Likewise, Cetux-CAR⁺ T cells demonstrated significantly more lysis of U87 (10:1 E:T ratio, p<0.0001) and U87low (10:1 E:T ratio, p<0.05) than Nimo-CAR⁺ T cells, but statistically similar specific lysis of U87med (10:1 E:T ratio, p>0.05) and U87high (10:1 E:T ratio, p>0.05) (FIG. 18E). In sum, these data show that activation of Nimo-CAR⁺ T cells is directly correlated to EGFR expression density on target. As a result, Cetux-CAR⁺ and Nimo-CAR⁺ T cells demonstrate equivalent T-cell activation in response to high EGFR density, but Nimo-CAR⁺ T cells demonstrate significantly reduced activation in response to low EGFR density.

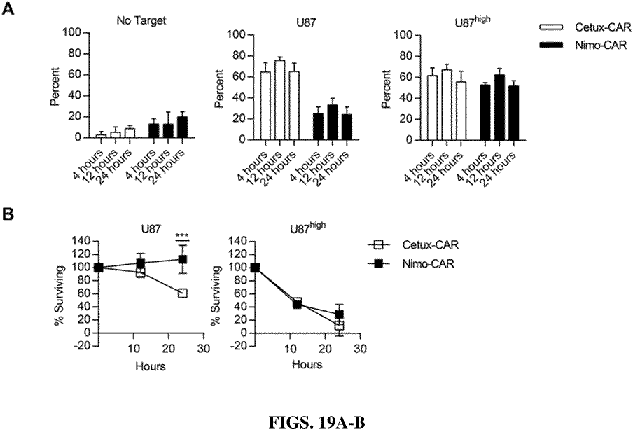

Because endogenous, low affinity T cell responses may require longer interaction with antigen to acquire effector function (Rossette et al., 2001), it was verified that the observed differences in T-cell activity between Cetux-CAR⁺ T cells and Nimo-CAR⁺ T cells was not due to a similar requirement for Nimo-CAR⁺ T cells. Extending interaction of CAR⁺ T cells with targets did not substantially increase cytokine production and did not alter the relationship of cytokine production between Cetux-CAR⁺ and Nimo-CAR⁺ CD8⁺ T cells (FIG. 19A). Similarly, the ability of Cetux-CAR⁺ and Nimo-CAR⁺ T cells to control growth of U87 and U87high over time was evaluated and it was found that Cetux-CAR⁺ and Nimo-CAR⁺ T cells demonstrated statistically similar ability to control the growth of U87high, resulting in 80% reduction in cell number relative to controls grown in the absence of CAR⁺ T cells (p>0.05). Cetux-CAR⁺ T cells controlled growth of U87 with endogenously low EGFR expression, resulting in 40% reduction in cell number relative to controls grown in the absence of CAR⁺ T cells. However, Nimo-CAR⁺ T cells demonstrated significantly less control of U87 growth, with no apparent reduction in cell number (p<0.001) (FIG. 19B). These data indicate that Nimo-CAR⁺ T cell activity in response to low EGFR on U87 is not improved by increasing interaction time of T cells with targets, making it unlikely that reduced activity of Nimo-CAR⁺ T cells is due to a requirement for prolonged interaction to activate T cells.

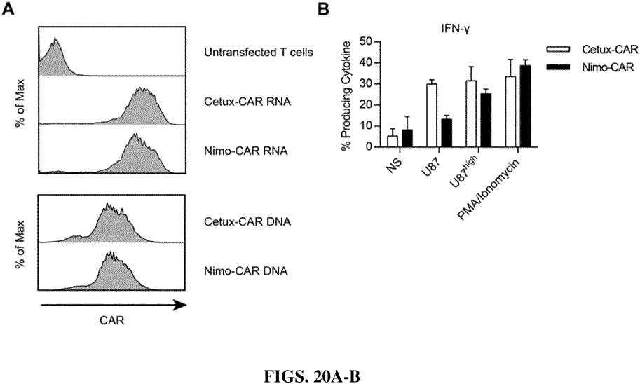

Expression of CAR above a minimum density is required for CAR-dependent T cell activation, and increasing density of CAR expression has been shown to impact sensitivity of CAR to antigen (Weijtens et al., 2000; Turatti et al., 2007). Therefore, to determine if expressing Nimo-CAR with higher density improves recognition of low EGFR density, it was sought to overexpress Cetux-CAR and Nimo-CAR in human primary T cells. Load of DNA in electroporation transfection is limited due to toxicity of DNA to cells, however, transfer of RNA is relatively non-toxic and more amenable to overexpression by increasing amount of CAR RNA transcript delivered. Therefore, Cetux-CAR and Nimo-CAR were in vitro transcribed as RNA species and electro-transferred into human primary T cells. RNA transfer resulted in 2-5 fold increased expression of CAR when compared to donor-matched DNA-modified T cells (FIG. 20A). Overexpression of CAR did not render Nimo-CAR⁺ T cells more sensitive to low EGFR density on U87 and both Cetux-CAR and Nimo-CAR demonstrated similar cytokine production in response to U87high (FIG. 20B). This indicates that increasing CAR density on Nimo-CAR⁺ T cells does not increase sensitivity to low EGFR density.

### Example 14 - Nimo-CAR⁺ T cells Have Reduced Activity in Response to Basal EGFR Levels on Normal Renal Epithelial Cells

To determine if Nimo-CAR⁺ T cells have reduced activation in response to low, basal EGFR levels on normal cells, the activity of Nimo-CAR⁺ T cells was evaluated in response to normal human renal cortical epithelial cells, HRCE. HRCE express ~15,000 molecules of EGFR per cell, lower than expression on tumor cell lines, including U87 (FIG. 21A). While Cetux-CAR⁺ T cells produced IFN-γ and TNF-α in response to HRCE, Nimo-CAR⁺ T cells produced significantly less IFN-γ or TNF-α in response to HRCE (IFN-γ, p<0.05; TNF-α, p<0.01) (FIG. 21B). In fact, Nimo-CAR⁺ T cells did not demonstrate significant production of IFN-γ or TNF-α above background production without stimulation (IFN-γ, p>0.05; TNF-α, p>0.05). Nimo-CAR⁺ T cells displayed less than 50% of the specific lysis executed by Cetux-CAR⁺ T cells in response to HRCE (Cetux-CAR=81.1±4.5%, Nimo-CAR=30.4±16.7%, mean±SD, n=3), which was significantly less (10:1 E:T ratio, p<0.001) (FIG. 21C). These findings indicate that Nimo-CAR⁺ T cells have reduced T-cell function in response to cells with very low EGFR density.

### Example 15 - Cetux-CAR⁺ T cells Proliferate Less Following Stimulation Than Nimo-CAR⁺ T Cells, but Do Not Have Increased Propensity for AICD

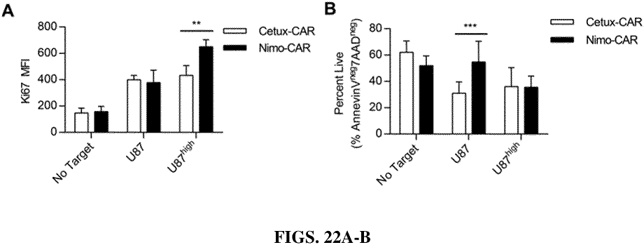

Strength of endogenous TCR signal, impacted by affinity of binding and antigen density, can influence proliferation of T cells in response to antigenic stimulus (Gottschalk et al., 2012; Gottschalk et al., 2010). To evaluate proliferative response of Cetux-CAR⁺ T cells and Nimo-CAR⁺ T cells following stimulation with antigen, intracellular expression of Ki-67 was measured by flow cytometry after two days of co-culture with U87 or U87high in absence of exogenous cytokines. In response to low EGFR density on U87, Cetux-CAR⁺ and Nimo-CAR⁺ T cells demonstrated statistically similar proliferation (p>0.05) (FIG. 22A). In response to U87high, Nimo-CAR⁺ T cells demonstrated increased proliferation over Cetux-CAR⁺ (p<0.01), which did not show any statistical difference in proliferation in response to U87 and U87high (p>0.05).

To determine if affinity of CAR or antigen density increases the propensity of CAR⁺ T cells to undergo AICD, Cetux-CAR⁺ and Nimo-CAR⁺ T cells were cocultured with U87 or U87high in the absence of exogenous cytokines and evaluated T-cell viability by annexin V and 7-AAD staining. In response to U87, Cetux-CAR⁺ T showed reduction in viability compared to unstimulated Cetux-CAR⁺ T cells, however, Nimo-CAR⁺ T cells did not show any appreciable change in viability (FIG. 22B). In response to U87high, Cetux-CAR⁺ and Nimo-CAR⁺ T cells demonstrated statistically similar reduction in viability relative to unstimulated CAR⁺ T cells (p>0.05). It was noted that Cetux-CAR⁺ T cells stimulated with U87high did not show any statistical difference in viability relative to Cetux-CAR⁺ T cells stimulate with U87 (p>0.05). These data suggest that antigen density impact induction of AICD for Nimo-CAR⁺ T cells, but not Cetux-CAR⁺ T cells, supporting previous data that activity of Nimo-CAR is dependent on antigen density. However, in response to high antigen density that is capable of Cetux-CAR⁺ T cells and Nimo-CAR⁺ T cell activation, affinity of scFv domain of CAR does not appear to impact the induction of AICD.

### Example 16 - Cetux-CAR⁺ T cells Demonstrate Enhanced Downregulation of CAR

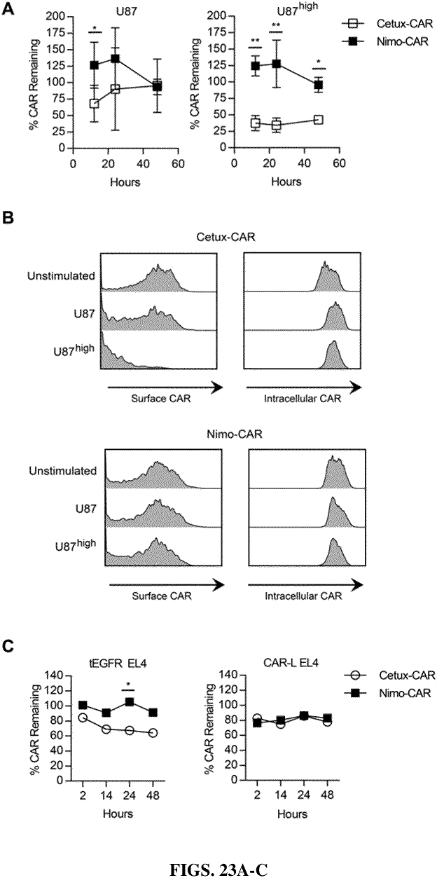

Endogenous TCR can be downregulated following interaction with antigen, and the degree of downregulation is influenced by the strength of TCR binding (Cai et al., 1997). Similary, CAR can be downregulated following interaction with antigen, but the effect of affinity on CAR downregulation is unknown (James et al., 2008; James et al., 2010). Therefore, it was sought to determine if Cetux-CAR⁺ T cells have a higher propensity for antigen-induced downregulation. To accomplish this, Cetux-CAR⁺T cells and Nimo-CAR⁺ T cells were co-cultured with U87 or U87high and monitored CAR expression relative to unstimulated controls. In response to low EGFR density on U87, Cetux-CAR expression was significantly less than Nimo-CAR after 12 hours of interaction (Cetux-CAR=68.0±27.8%, Nimo-CAR=126.5±34.9%, mean±SD, n=3) (p<0.05) (FIG. 23A, left panel). By 48 hours of interaction with low density EGFR, Cetux-CAR returned to the T-cell surface, and Cetux-CAR and Nimo-CAR were expressed in a statistically similar proportion of T cells (Cetux-CAR=95.5±40.7, Nimo-CAR=94.4±11.8%, mean±SD, n=3) (p>0.05). In response to high EGFR density on U87high, expression of Cetux-CAR was significantly reduced relative to Nimo-CAR, which showed no appreciable downregulation after 12 hours of interaction (Cetux-CAR=37.4±11.5%, Nimo-CAR=124.4±15.3%, mean±SD, n=3) (p<0.01) (12 hrs, p<0.01; 24 hrs, p<0.01; 48 hrs, p<0.05) (FIG. 23A, right panel). However, in contrast to stimulation with low EGFR density, Cetux-CAR did not recover surface expression after 48 hours of interaction and remained statistically reduced relative to Nimo-CAR expression (Cetux-CAR=42.6±5.9%, Nimo-CAR=95.7±11.6%, mean±SD, n=3)(p<0.05). Cetux-CAR and Nimo-CAR were both detected intracellularly following stimulation, even when Cetux-CAR was reduced from the T-cell surface, signifying that reduced CAR expression was due to internalization of CAR and not outgrowth of genetically unmodified T cells (FIG. 23B). In response to CAR-dependent, scFv-independent stimulation by CAR-L⁺ EL4, Cetux-CAR and Nimo-CAR showed mild and statistically similar downregulation of ~20% (FIG. 23C). Similar to previous results, Cetux-CAR showed slight downregulation in response to tEGFR⁺ EL4, whereas Nimo-CAR showed no appreciable downregulation. In sum, these data show that Cetux-CAR demonstrates more rapid and prolonged downregulation relative to Nimo-CAR that is dependent on interaction of the scFv domain of CAR with antigen and antigen density.

### Example 17 - Cetux-CAR⁺ T cells Have Reduced Response to Re-Challenge With Antigen

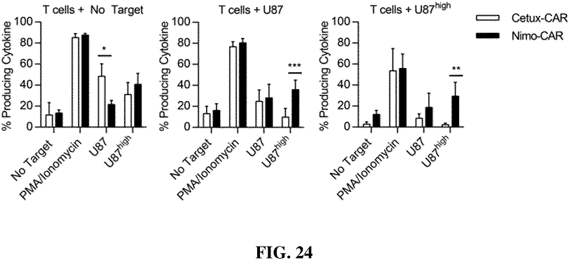

Strength of prior stimulus in endogenous CD8⁺ T cell responses can correlated with T-cell response upon re-challenge with antigen (Lim et al., 2002). Therefore, the ability of Cetux-CAR⁺ and Nimo-CAR⁺ T cells to respond to antigen re-challenge was evaluated. CAR⁺ T cells were co-cultured with U87 or U87high for 24 hours, then harvested and rechallenged with U87 or U87high to assess production of IFN-γ. Following initial challenge with U87 and U87high, Cetux-CAR⁺ T cells had reduced production of IFN-γ in response to rechallenge with both U87 and U87high (FIG. 24) However, after initial challenge with U87 or U87high, Nimo-CAR⁺ T cells retained IFN-γ production in response to re-challenge with U87 and U87high. As a result, Nimo-CAR⁺ T cells demonstrated statistically similar IFN-γ production in response to U87 (p>0.05) and statistically more IFN-γ in response to rechallenge with U87high (initial challenge with U87, p<0.001; initial challenge with U87high p<0.01). This is in contrast to IFN-γ production in response to initial challenge, in which Nimo-CAR⁺ T cells produce less IFN-γ in response to U87(p<0.05) and demonstrate statistically similar IFN-γ production in response to U87high (p>0.05). Thus, while Nimo-CAR⁺ T cells retain their ability to recognize and respond to antigen, Cetux-CAR⁺ T cells have reduced capacity to respond to subsequent encounter with antigen, which is likely to be at least partially due to downregulation of CAR and may indicate increased propensity for functional exhaustion of Cetux-CAR⁺ T cells after initial antigen exposure.

### Example 18 - Establishment of an Intracranial Glioma Model Using U87 Cells in NSG Mice

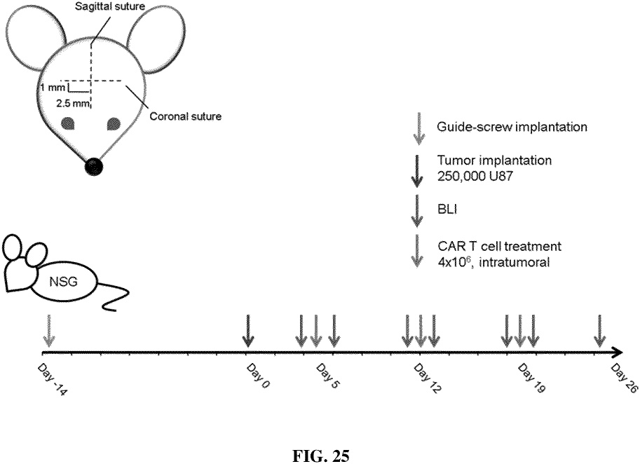

To evaluate anti-tumor efficacy of Cetux-CAR⁺ T cells and Nimo-CAR⁺ T cells in vivo, an intracranial glioma xenograft of U87 cells modified to express firefly luciferase (ffLuc) reporter for serial, non-invasive imaging of relative tumor burden by bioluminescence (BLI) was established. The previously described guide-screw method was adopted for directed infusion of tumor and T cells into precise coordinates (Lal et al., 2000). The guide screw was implanted into the right frontal lobe of the cranium of NOD/Scid/IL2Rg-/- (NSG) mice and mice recovered for two weeks (FIG. 25). A timeline from guide screw implantation through T-cell treatment and evaluation of relative tumor burden by BLI is depicted in FIG. 25. 250,000 U87 cells with endogenously low EGFR or intermediate EGFR expression through enforced expression of tEGFR were injected through the center of the guide screw at depth of 2.5 mm. Mice were imaged prior to T-cell treatment to evaluate tumor burden and mice were stratified to evenly distribute tumor burden into three groups: mice to receive no treatment, Cetux-CAR⁺ T cells, or Nimo-CAR⁺ T cells. Five days after injection of tumor, the initial dose of 4x10⁶ T cells was injected through the center of the guide screw. Subsequent T cell doses were administered through the guide screw weekly for a total of three T-cell doses. Measurement of BLI six days after each T-cell treatment was used to assess relative tumor burden. Following treatment, mice were evaluated for end point criteria, including rapid weight loss of greater than 5% of body mass in a 24 hour period, progressive weight loss of more than 25% of body mass, or obvious clinical signs of illness, including ataxia, labored respiration, and hind-limb paralysis. Mice were sacrificed when end-point criteria were met, suggesting imminent animal death, and survival of Cetux-CAR⁺ T cell treated mice and Nimo-CAR⁺ T cell treated mice relative to mice receiving no treatment was assessed.

### Example 19 - Nimo-CAR⁺ T cells Inhibit Growth of Xenografts With Moderate EGFR Density similar to Cetux-CAR⁺ T cells, but Without T-cell Related Toxicity

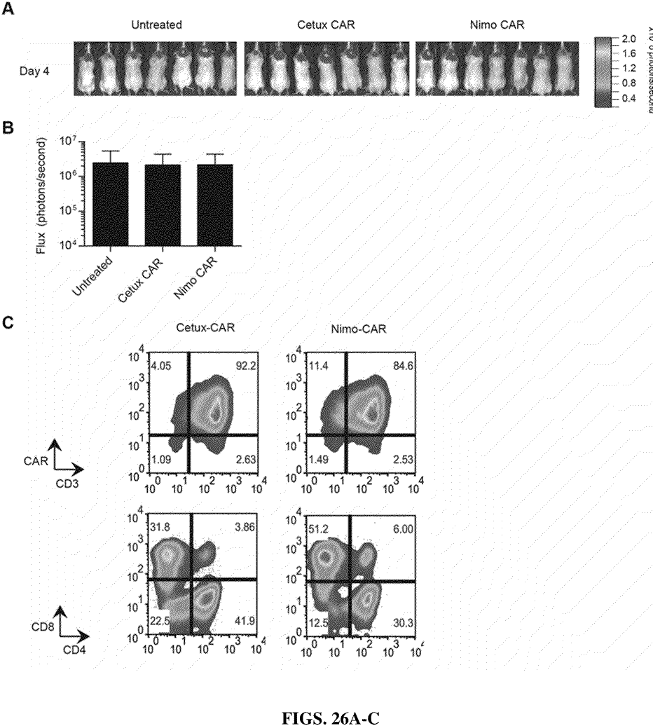

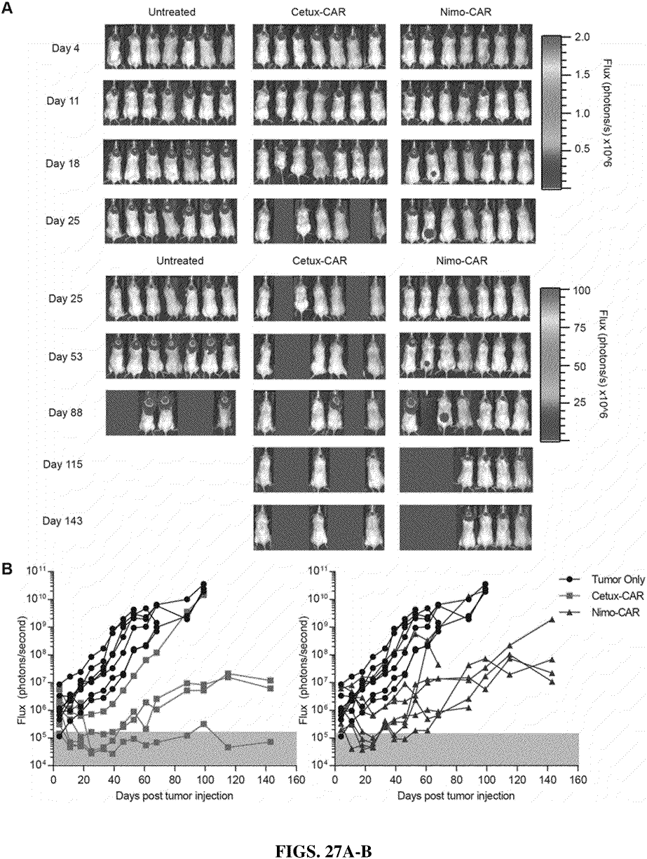

Four days after injection of U87med, mice were imaged by BLI to assess tumor burden (FIG. 26A). Mice were distributed into three groups to evenly distribute relative tumor burden and then randomly assigned treatment: no treatment, Cetux-CAR⁺ T cells, or Nimo-CAR⁺ T cells (FIG. 26B). On the day of T-cell treatment, CAR⁺ T cells that had undergone 3 rounds of stimulation and numeric expansion on EGFR⁺ aAPC were phenotyped by flow cytometry to determine expression of CAR and ratio of CD8⁺ and CD4⁺ T cells (FIG. 26C). CAR expression was similar between Cetux-CAR⁺ T cells and Nimo-CAR⁺ T cells (92% and 85%, respectively). Both Cetux-CAR⁺ and Nimo-CAR⁺ T cells contained a mixture of CD4⁺ and CD8⁺ T cells, however, Cetux-CAR⁺ T cells contained about 20% fewer CD8⁺ T cells than Nimo-CAR⁺ T cells (31.8% and 51.2%, respectively). Cetux-CAR⁺ T cells and Nimo-CAR⁺ T cells were both capable of inhibiting tumor growth as assayed by BLI (day 18; Cetux-CAR, p<0.01 and Nimo-CAR, p<0.05) (FIGS. 27A,B). There was no difference between the ability of Cetux-CAR⁺ T cells and Nimo-CAR⁺ T cells to control tumor growth (p>0.05). Reduced tumor burden assessed by BLI was evident in 3/7 mice treated with Cetux-CAR⁺ T cells and 4/7 mice treated with Nimo-CAR⁺ T cells past 100 days post-tumor injection, when all mice which did not receive treatment had succumbed to disease.

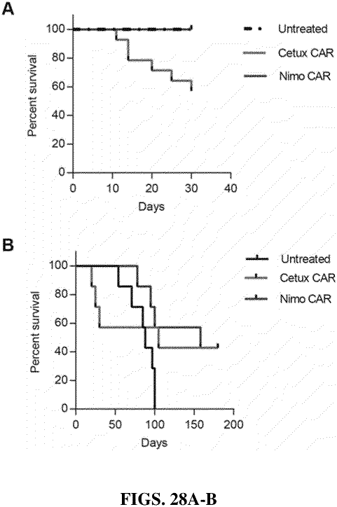

Cetux-CAR⁺ T-cell treated mice showed significant toxicity resulting in death of 6/14 mice within 7 days of T-cell treatment from two independent experiments (p=0.0006) (FIG. 28A). Overall, Cetux-CAR⁺ T-cell treatment did not statistically improve survival compared to untreated mice, possibly due to early deaths soon after T-cell treatment (untreated median survival = 88 days, Cetux-CAR median survival = 105 days, p=0.19) (FIG. 28B). Interestingly, the survival curve depicts an inflection point, before which Cetux-CAR⁺ T-cell treatment results in reduced survival compared to untreated mice, and after which mice surviving initial T-cell toxicity show improved survival. When only considering mice surviving initial T-cell related toxicity, Cetux-CAR⁺ T cells improve in ¾ mice, relative to untreated mice (p=0.0065). In contrast, Nimo-CAR⁺ T cells mediate effective tumor regression and extend survival in 4/7 of mice without any noted toxicity (untreated median survival = 88 days, Nimo-CAR median survival = 158 days, p=0.0269). These results indicate that Cetux-CAR⁺ T cells and Nimo-CAR⁺ T cells are effective at controlling growth of tumor with intermediate antigen density, however Cetux-CAR⁺ T cells demonstrate notable toxicity soon after T-cell treatment.

### Example 20 - Cetux-CAR⁺ T cells, but not Nimo-CAR⁺ T cells, Inhibit Growth of Xenografts With low EGFR Density

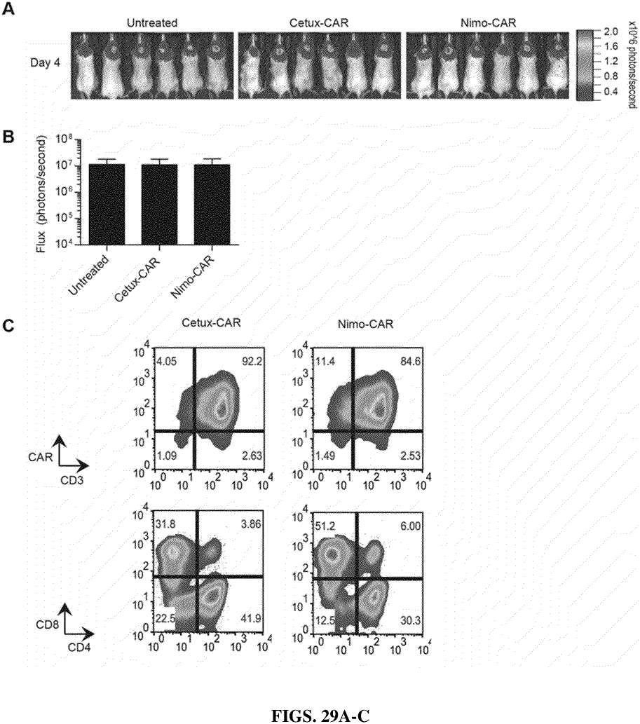

Mice were injected with U87, then four days later relative tumor burden was assessed by BLI (FIG. 29A). Relative tumor burden was evenly distributed into three groups and randomly assigned treatment: no treatment, Cetux-CAR⁺ T cells, or Nimo-CAR⁺ T cells (FIG. 29B). On the day of T cell treatment, CAR⁺ T cells that had undergone 3 rounds of stimulation and numeric expansion on EGFR⁺ aAPC were phenotyped by flow cytometry to determine expression of CAR and ratio of CD8⁺ and CD4⁺ T cells (FIG. 29C). CAR expression was similar between Cetux-CAR⁺ T cells and Nimo-CAR⁺ T cells (92% and 85%, respectively). Both Cetux-CAR⁺ and Nimo-CAR⁺ T cells contained a mixture of CD4⁺ and CD8⁺ T cells, however, Cetux-CAR⁺ T cells contained about 20% fewer CD8⁺ T cells than Nimo-CAR⁺ T cells (31.8% and 51.2%, respectively).

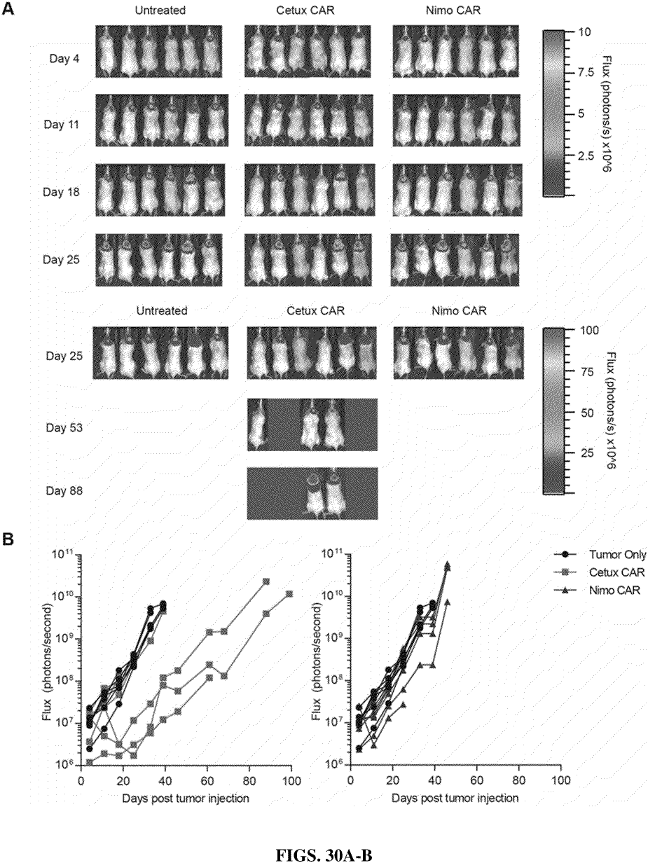

Mice received T-cell treatment and tumor was assessed by BLI as previously described (FIG. 25). Treatment of mice with Cetux-CAR⁺ T cells resulted in significant reduction of tumor burden compared to untreated mice (day 25, p< 0.01) (FIGS. 30A and 30B). In contrast, treatment with Nimo-CAR⁺ T cells did not significantly reduce tumor burden compared to untreated mice (Nimo-CAR, p>0.05). Reduced tumor burden in mice treated with Cetux-CAR⁺ T cells was transient, however, and following cessation of T-cell treatment, tumors resumed growth.

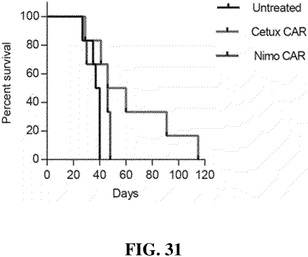

Cetux-CAR⁺ T cell treatment significantly extended survival in 3/6 mice compared to mice receiving no treatment (untreated median survival = 38.5 days, Cetux-CAR median survival = 53 days, p=0.0150) (FIG. 31). In contrast, treatment with Nimo-CAR⁺ T cells did not significantly improve survival (untreated median survival 38.5 days, Nimo-CAR median survival 46 days, p=0.0969). These data indicate that while Cetux CAR T cells are effective against low antigen density, Nimo-CAR⁺ T cells do not efficiently recognize low density EGFR expression.

### REFERENCES

The following references, to the extent that they provide exemplary procedural or other details supplementary to those set forth herein, are specifically incorporated herein by reference.
U.S. Pat. 4,690,915
U.S. Pat. 6,225,042
U.S. Pat. 6,355,479
U.S. Pat. 6,362,001
U.S. Pat. 6,410,319
U.S. Pat. 6,489,458
U.S. Pat. 6,790,662
U.S. Pat. 7,109,304
U.S. Pat. Application Publication No. 2009/0004142
U.S. Pat. Application Publication No. 2009/0017000
International Publication No. WO2007/103009
International Publication No. WO2012/100346

Adams, G. P., R. Schier, A. M. McCall, H. H. Simmons, E. M. Horak, R. K. Alpaugh, J. D. Marks, and L. M. Weiner. 2001. High affinity restricts the localization and tumor penetration of single-chain fv antibody molecules. Cancer research 61:4750-4755.

Ahmed, N., V. S. Salsman, Y. Kew, D. Shaffer, S. Powell, Y. J. Zhang, R. G. Grossman, H. E. Heslop, and S. Gottschalk. 2010. HER2-specific T cells target primary glioblastoma stem cells and induce regression of autologous experimental tumors. Clinical cancer research : an official journal of the American Association for Cancer Research 16:474-485.

Aleksic, M., O. Dushek, H. Zhang, E. Shenderov, J. L. Chen, V. Cerundolo, D. Coombs, and P. A. van der Merwe. 2010. Dependence of T cell antigen recognition on T cell receptor-peptide MHC confinement time. Immunity 32:163-174.

Altenschmidt, U. et al., J.Immunol. 159:5509, 1997.

Audic, S., and J. M. Claverie. 1997. The significance of digital gene expression profiles. Genome research 7:986-995.

Barker, F. G., 2nd, M. L. Simmons, S. M. Chang, M. D. Prados, D. A. Larson, P. K. Sneed, W. M. Wara, M. S. Berger, P. Chen, M. A. Israel, and K. D. Aldape. 2001. EGFR overexpression and radiation response in glioblastoma multiforme. International journal of radiation oncology, biology, physics 51:410-418.

Barrett, D. M., D. T. Teachey, and S. A. Grupp. 2014. Toxicity management for patients receiving novel T-cell engaging therapies. Current opinion in pediatrics 26:43-49.

Barrett, D. M., X. Liu, S. Jiang, C. H. June, S. A. Grupp, and Y. Zhao. 2013. Regimen-specific effects of RNA-modified chimeric antigen receptor T cells in mice with advanced leukemia. Human gene therapy 24:717-727.

Barrett, D. M., Y. Zhao, X. Liu, S. Jiang, C. Carpenito, M. Kalos, R. G. Carroll, C. H. June, and S. A. Grupp. 2011. Treatment of advanced leukemia in mice with mRNA engineered T cells. Human gene therapy 22:1575-1586.

Barthel and Goldfeld, J. Immunol., 171:3612-3619, 2003.

Boczkowski, D., S. K. Nair, J. H. Nam, H. K. Lyerly, and E. Gilboa. 2000. Induction of tumor immunity and cytotoxic T lymphocyte responses using dendritic cells transfected with messenger RNA amplified from tumor cells. Cancer research 60:1028-1034.

Bourgeois, C., H. Veiga-Fernandes, A. M. Joret, B. Rocha, and C. Tanchot. 2002. CD8 lethargy in the absence of CD4 help. European journal of immunology 32:2199-2207.

Brentjens, R. J., I. Riviere, J. H. Park, M. L. Davila, X. Wang, J. Stefanski, C. Taylor, R. Yeh, S. Bartido, O. Borquez-Ojeda, M. Olszewska, Y. Bernal, H. Pegram, M. Przybylowski, D. Hollyman, Y. Usachenko, D. Pirraglia, J. Hosey, E. Santos, E. Halton, P. Maslak, D. Scheinberg, J. Jurcic, M. Heaney, G. Heller, M. Frattini, and M. Sadelain. 2011. Safety and persistence of adoptively transferred autologous CD19-targeted T cells in patients with relapsed or chemotherapy refractory B-cell leukemias. Blood 118:4817-4828.

Brentjens, R. J., M. L. Davila, I. Riviere, J. Park, X. Wang, L. G. Cowell, S. Bartido, J. Stefanski, C. Taylor, M. Olszewska, O. Borquez-Ojeda, J. Qu, T. Wasielewska, Q. He, Y. Bernal, I. V. Rijo, C. Hedvat, R. Kobos, K. Curran, P. Steinherz, J. Jurcic, T. Rosenblat, P. Maslak, M. Frattini, and M. Sadelain. 2013. CD19-targeted T cells rapidly induce molecular remissions in adults with chemotherapy-refractory acute lymphoblastic leukemia. Science translational medicine 5:177ra138.

Bridgeman, J. S., R. E. Hawkins, S. Bagley, M. Blaylock, M. Holland, and D. E. Gilham. 2010. The optimal antigen response of chimeric antigen receptors harboring the CD3zeta transmembrane domain is dependent upon incorporation of the receptor into the endogenous TCR/CD3 complex. J Immunol 184:6938-6949.

Brocker T. Karjalainen K. Adoptive tumor immunity mediated by lymphocytes bearing modified antigen-specific receptors. Adv. Immunol. 1998;68:257-269.

Budde, L. E., C. Berger, Y. Lin, J. Wang, X. Lin, S. E. Frayo, S. A. Brouns, D. M. Spencer, B. G. Till, M. C. Jensen, S. R. Riddell, and O. W. Press. 2013. Combining a CD20 Chimeric Antigen Receptor and an Inducible Caspase 9 Suicide Switch to Improve the Efficacy and Safety of T Cell Adoptive Immunotherapy for Lymphoma. PloS one 8:e82742.

Cai, Z., H. Kishimoto, A. Brunmark, M. R. Jackson, P. A. Peterson, and J. Sprent. 1997. Requirements for peptide-induced T cell receptor downregulation on naive CD8⁺ T cells. The Journal of experimental medicine 185:641-651.

Chan, D. A., P. D. Sutphin, S. E. Yen, and A. J. Giaccia. 2005. Coordinate regulation of the oxygen-dependent degradation domains of hypoxia-inducible factor 1 alpha. Molecular and cellular biology 25:6415-6426.

Chervin, A. S., J. D. Stone, C. M. Soto, B. Engels, H. Schreiber, E. J. Roy, and D. M. Kranz. 2013. Design of T-cell receptor libraries with diverse binding properties to examine adoptive T-cell responses. Gene therapy 20:634-644.

Chmielewski, M., A. Hombach, C. Heuser, G. P. Adams, and H. Abken. 2004. T cell activation by antibody-like immunoreceptors: increase in affinity of the single-chain fragment domain above threshold does not increase T cell activation against antigen-positive target cells but decreases selectivity. J Immunol 173:7647-7653.

Comprehensive genomic characterization defines human glioblastoma genes and core pathways. Nature 455:1061-1068, 2008.

Cooper et al., Good T cells for bad B cells, Blood, 119:2700-2702, 2012.

Corse, E., R. A. Gottschalk, M. Krogsgaard, and J. P. Allison. 2010. Attenuated T cell responses to a high-potency ligand in vivo. PLoS biology 8.

Davies, J. K., H. Singh, H. Huls, D. Yuk, D. A. Lee, P. Kebriaei, R. E. Champlin, L. M. Nadler, E. C. Guinan, and L. J. Cooper. 2010. Combining CD19 redirection and alloanergization to generate tumor-specific human T cells for allogeneic cell therapy of B-cell malignancies. Cancer research 70:3915-3924.

Di Stasi, A., B. De Angelis, C. M. Rooney, L. Zhang, A. Mahendravada, A. E. Foster, H. E. Heslop, M. K. Brenner, G. Dotti, and B. Savoldo. 2009. T lymphocytes coexpressing CCR4 and a chimeric antigen receptor targeting CD30 have improved homing and antitumor activity in a Hodgkin tumor model. Blood 113:6392-6402.

Di Stasi, A., S. K. Tey, G. Dotti, Y. Fujita, A. Kennedy-Nasser, C. Martinez, K. Straathof, E. Liu, A. G. Durett, B. Grilley, H. Liu, C. R. Cruz, B. Savoldo, A. P. Gee, J. Schindler, R. A. Krance, H. E. Heslop, D. M. Spencer, C. M. Rooney, and M. K. Brenner. 2011. Inducible apoptosis as a safety switch for adoptive cell therapy. The New England journal of medicine 365:1673-1683.

Eisen, M. B., P. T. Spellman, P. O. Brown, and D. Botstein. 1998. Cluster analysis and display of genome-wide expression patterns. Proceedings of the National Academy of Sciences of the United States of America 95:14863-14868.

Engels, B., A. S. Chervin, A. J. Sant, D. M. Kranz, and H. Schreiber. 2012. Long-term persistence of CD4(+) but rapid disappearance of CD8(+) T cells expressing an MHC class I-restricted TCR of nanomolar affinity. Molecular therapy : the journal of the American Society of Gene Therapy 20:652-660.

Ertl H.C. Zaia J. Rosenberg S.A., et al. Considerations for the clinical application of chimeric antigen receptor T cells: observations from a recombinant DNA Advisory Committee Symposium held Jun. 15, 2010. Cancer Res. 2011;71:3175-3181.

Eshhar Z. Tumor-specific T-bodies: towards clinical application. Cancer Immunol. Immunother. 1997;45:131-136.

Eshhar, Z. et al., Proc.Natl.Acad.Sci.U.S.A. 90:720, 1993.

Fedorov, V. D., M. Themeli, and M. Sadelain. 2013. PD-1- and CTLA-4-Based Inhibitory Chimeric Antigen Receptors (iCARs) Divert Off-Target Immunotherapy Responses. Science translational medicine 5:215ra172.

Fitzer-Attas et al., J. Immunol., 160:145-154, 1998.

Galanis, E., J. Buckner, D. Kimmel, R. Jenkins, B. Alderete, J. O’Fallon, C. H. Wang, B. W. Scheithauer, and C. D. James. 1998. Gene amplification as a prognostic factor in primary and secondary high-grade malignant gliomas. International journal of oncology 13:717-724.

Garrido, G., I. A. Tikhomirov, A. Rabasa, E. Yang, E. Gracia, N. Iznaga, L. E. Fernandez, T. Crombet, R. S. Kerbel, and R. Perez. 2011. Bivalent binding by intermediate affinity of nimotuzumab: a contribution to explain antibody clinical profile. Cancer biology & therapy 11:373-382.

Gattinoni, L., C. A. Klebanoff, and N. P. Restifo. 2012. Paths to stemness: building the ultimate antitumour T cell. Nature reviews. Cancer 12:671-684.

Gattinoni, L., X. S. Zhong, D. C. Palmer, Y. Ji, C. S. Hinrichs, Z. Yu, C. Wrzesinski, A. Boni, L. Cassard, L. M. Garvin, C. M. Paulos, P. Muranski, and N. P. Restifo. 2009. Wnt signaling arrests effector T cell differentiation and generates CD8⁺ memory stem cells. Nature medicine 15:808-813.

Geginat, J., A. Lanzavecchia, and F. Sallusto. 2003. Proliferation and differentiation potential of human CD8⁺ memory T-cell subsets in response to antigen or homeostatic cytokines. Blood 101:4260-4266.

Gottschalk, R. A., E. Corse, and J. P. Allison. 2010. TCR ligand density and affinity determine peripheral induction of Foxp3 in vivo. The Journal of experimental medicine 207:1701-1711.

Gottschalk, R. A., M. M. Hathorn, H. Beuneu, E. Corse, M. L. Dustin, G. Altan-Bonnet, and J. P. Allison. 2012. Distinct influences of peptide-MHC quality and quantity on in vivo T-cell responses. Proceedings of the National Academy of Sciences of the United States of America 109:881-886.

Govern, C. C., M. K. Paczosa, A. K. Chakraborty, and E. S. Huseby. 2010. Fast on-rates allow short dwell time ligands to activate T cells. Proceedings of the National Academy of Sciences of the United States of America 107:8724-8729.

Gross et al., FASEB J 6:3370, 1992.

Grupp, S. A., M. Kalos, D. Barrett, R. Aplenc, D. L. Porter, S. R. Rheingold, D. T. Teachey, A. Chew, B. Hauck, J. F. Wright, M. C. Milone, B. L. Levine, and C. H. June. 2013. Chimeric antigen receptor-modified T cells for acute lymphoid leukemia. The New England journal of medicine 368:1509-1518.

Hegde, M., A. Corder, K. K. Chow, M. Mukherjee, A. Ashoori, Y. Kew, Y. J. Zhang, D. S. Baskin, F. A. Merchant, V. S. Brawley, T. T. Byrd, S. Krebs, M. F. Wu, H. Liu, H. E. Heslop, S. Gottachalk, E. Yvon, and N. Ahmed. 2013. Combinational targeting offsets antigen escape and enhances effector functions of adoptively transferred T cells in glioblastoma. Molecular therapy : the journal of the American Society of Gene Therapy 21:2087-2101.

Hekele, A. et al., Int.J.Cancer 68:232, 1996.

Hemmer, B., I. Stefanova, M. Vergelli, R. N. Germain, and R. Martin. 1998. Relationships among TCR ligand potency, thresholds for effector function elicitation, and the quality of early signaling events in human T cells. J Immunol 160:5807-5814.

Hirsch, F. R., M. Varella-Garcia, and F. Cappuzzo. 2009. Predictive value of EGFR and HER2 overexpression in advanced non-small-cell lung cancer. Oncogene 28 Suppl 1:S32-37.

Holler, P. D., and D. M. Kranz. 2003. Quantitative analysis of the contribution of TCR/pepMHC affinity and CD8 to T cell activation. Immunity 18:255-264.

Hu, X., W. Miao, Y. Zou, W. Zhang, Y. Zhang, and H. Liu. 2013. Expression of p53, epidermal growth factor receptor, Ki-67 and O-methylguanine-DNA methyltransferase in human gliomas. Oncology letters 6:130-134.

Huang, J., V. I. Zarnitsyna, B. Liu, L. J. Edwards, N. Jiang, B. D. Evavold, and C. Zhu. 2010. The kinetics of two-dimensional TCR and pMHC interactions determine T-cell responsiveness. Nature 464:932-936.

Hudecek, M., M. T. Lupo-Stanghellini, P. L. Kosasih, D. Sommermeyer, M. C. Jensen, C. Rader, and S. R. Riddell. 2013. Receptor affinity and extracellular domain modifications affect tumor recognition by ROR1-specific chimeric antigen receptor T cells. Clinical cancer research : an official journal of the American Association for Cancer Research 19:3153-3164.

Huppa, J. B., M. Axmann, M. A. Mortelmaier, B. F. Lillemeier, E. W. Newell, M. Brameshuber, L. O. Klein, G. J. Schutz, and M. M. Davis. 2010. TCR-peptide-MHC interactions in situ show accelerated kinetics and increased affinity. Nature 463:963-967.

Hurton, L. V. 2014. Tethered IL-15 to augment the therapeutic potential of T cells expressing chimeric antigen receptor: Maintaining memory potential, persistence, and antitumor activity. (Doctoral Dissertation) The University of Texas Health Science Center at Houston.

Hwu et al., Cancer Res. 55:3369, 1995.

Hynes, N. E., and H. A. Lane. 2005. ERBB receptors and cancer: the complexity of targeted inhibitors. Nature reviews. Cancer 5:341-354.

James, S. E., P. D. Greenberg, M. C. Jensen, Y. Lin, J. Wang, B. G. Till, A. A. Raubitschek, S. J. Forman, and O. W. Press. 2008. Antigen sensitivity of CD22-specific chimeric TCR is modulated by target epitope distance from the cell membrane. J Immunol 180:7028-7038.

James, S. E., P. D. Greenberg, M. C. Jensen, Y. Lin, J. Wang, L. E. Budde, B. G. Till, A. A. Raubitschek, S. J. Forman, and O. W. Press. 2010. Mathematical modeling of chimeric TCR triggering predicts the magnitude of target lysis and its impairment by TCR downmodulation. J Immunol 184:4284-4294.

Janicki, C. N., S. R. Jenkinson, N. A. Williams, and D. J. Morgan. 2008. Loss of CTL function among high-avidity tumor-specific CD8⁺ T cells following tumor infiltration. Cancer research 68:2993-3000.

Jena, B., G. Dotti, and L. J. Cooper. 2010. Redirecting T-cell specificity by introducing a tumor-specific chimeric antigen receptor. Blood 116:1035-1044.

Kalergis, A. M., N. Boucheron, M. A. Doucey, E. Palmieri, E. C. Goyarts, Z. Vegh, I. F. Luescher, and S. G. Nathenson. 2001. Efficient T cell activation requires an optimal dwell-time of interaction between the TCR and the pMHC complex. Nature immunology 2:229-234.

Kalos, M., B. L. Levine, D. L. Porter, S. Katz, S. A. Grupp, A. Bagg, and C. H. June. 2011. T cells with chimeric antigen receptors have potent antitumor effects and can establish memory in patients with advanced leukemia. Science translational medicine 3:95ra73.

Kamphorst, A. O., and R. Ahmed. 2013. CD4 T-cell immunotherapy for chronic viral infections and cancer. Immunotherapy 5:975-987.

Kersh, G. J., E. N. Kersh, D. H. Fremont, and P. M. Allen. 1998. High- and low-potency ligands with similar affinities for the TCR: the importance of kinetics in TCR signaling. Immunity 9:817-826.

Kloss, C. C., M. Condomines, M. Cartellieri, M. Bachmann, and M. Sadelain. 2013. Combinatorial antigen recognition with balanced signaling promotes selective tumor eradication by engineered T cells. Nature biotechnology 31:71-75.

Kochenderfer, J. N., M. E. Dudley, S. A. Feldman, W. H. Wilson, D. E. Spaner, I. Maric, M. Stetler-Stevenson, G. Q. Phan, M. S. Hughes, R. M. Sherry, J. C. Yang, U. S. Kammula, L. Devillier, R. Carpenter, D. A. Nathan, R. A. Morgan, C. Laurencot, and S. A. Rosenberg. 2012. B-cell depletion and remissions of malignancy along with cytokine-associated toxicity in a clinical trial of anti-CD19 chimeric-antigen-receptor-transduced T cells. Blood 119:2709-2720.

Kochenderfer, J. N., W. H. Wilson, J. E. Janik, M. E. Dudley, M. Stetler-Stevenson, S. A. Feldman, I. Maric, M. Raffeld, D. A. Nathan, B. J. Lanier, R. A. Morgan, and S. A. Rosenberg. 2010. Eradication of B-lineage cells and regression of lymphoma in a patient treated with autologous T cells genetically engineered to recognize CD19. Blood 116:4099-4102.

Kohn D.B. Dotti G. Brentjens R., et al. CARs on track in the clinic. Mol. Ther. 2011;19:432-438.

Kowolik, C. M., M. S. Topp, S. Gonzalez, T. Pfeiffer, S. Olivares, N. Gonzalez, D. D. Smith, S. J. Forman, M. C. Jensen, and L. J. Cooper. 2006. CD28 costimulation provided through a CD19-specific chimeric antigen receptor enhances in vivo persistence and antitumor efficacy of adoptively transferred T cells. Cancer research 66:10995-11004.

Kumar, R., M. Ferez, M. Swamy, I. Arechaga, M. T. Rejas, J. M. Valpuesta, W. W. Schamel, B. Alarcon, and H. M. van Santen. 2011. Increased sensitivity of antigen-experienced T cells through the enrichment of oligomeric T cell receptor complexes. Immunity 35:375-387.

Lacunza, E., M. Baudis, A. G. Colussi, A. Segal-Eiras, M. V. Croce, and M. C. Abba. 2010. MUC1 oncogene amplification correlates with protein overexpression in invasive breast carcinoma cells. Cancer genetics and cytogenetics 201:102-110.

Lal, S., M. Lacroix, P. Tofilon, G. N. Fuller, R. Sawaya, and F. F. Lang. 2000. An implantable guide-screw system for brain tumor studies in small animals. Journal of neurosurgery 92:326-333.

Lamers, C. H., S. Sleijfer, S. van Steenbergen, P. van Elzakker, B. van Krimpen, C. Groot, A. Vulto, M. den Bakker, E. Oosterwijk, R. Debets, and J. W. Gratama. 2013. Treatment of metastatic renal cell carcinoma with CAIX CAR-engineered T cells: clinical evaluation and management of on-target toxicity. Molecular therapy : the journal of the American Society of Gene Therapy 21:904-912.

Lanitis, E., M. Poussin, A. W. Klattenhoff, D. Song, R. Sandaltzopoulos, C. H. June, and D. J. Powell, Jr. 2013. Chimeric antigen receptor T cells with dissociated signaling domains exhibit focused anti-tumor activity with reduced potential for toxicity. Cancer immunology research 1.

Lim, D. G., P. Hollsberg, and D. A. Hafler. 2002. Strength of prior stimuli determines the magnitude of secondary responsiveness in CD8⁺ T cells. Cellular immunology 217:36-46.

Little, S. E., S. Popov, A. Jury, D. A. Bax, L. Doey, S. Al-Sarraj, J. M. Jurgensmeier, and C. Jones. 2012. Receptor tyrosine kinase genes amplified in glioblastoma exhibit a mutual exclusivity in variable proportions reflective of individual tumor heterogeneity. Cancer research 72:1614-1620.

Marodon et al., Blood, 101:3416-3423, 2003.

Mateo et al. Immunotechnology, 3(1):71-81, 1997.

Maus, M. V., A. R. Haas, G. L. Beatty, S. M. Albelda, B. L. Levine, X. Liu, Y. Zhao, M. Kalos, and C. H. June. 2013. T cells expressing chimeric antigen receptors can cause anaphylaxis in humans. Cancer immunology research 1:26-31.

McKeithan, T. W. 1995. Kinetic proofreading in T-cell receptor signal transduction. Proceedings of the National Academy of Sciences of the United States of America 92:5042-5046.

Moon, E. K., C. Carpenito, J. Sun, L. C. Wang, V. Kapoor, J. Predina, D. J. Powell, Jr., J. L. Riley, C. H. June, and S. M. Albelda. 2011. Expression of a functional CCR2 receptor enhances tumor localization and tumor eradication by retargeted human T cells expressing a mesothelin-specific chimeric antibody receptor. Clinical cancer research : an official journal of the American Association for Cancer Research 17:4719-4730.

Morgan, R. A., J. C. Yang, M. Kitano, M. E. Dudley, C. M. Laurencot, and S. A. Rosenberg. 2010. Case report of a serious adverse event following the administration of T cells transduced with a chimeric antigen receptor recognizing ERBB2. Molecular therapy : the journal of the American Society of Gene Therapy 18:843-851.

Moritz, D. et al., Proc.Natl.Acad.Sci.U.S.A. 91:4318, 1994.

Muranski, P., and N. P. Restifo. 2009. Adoptive immunotherapy of cancer using CD4(+) T cells. Current opinion in immunology 21:200-208.

Mutsaers, A. J., G. Francia, S. Man, C. R. Lee, J. M. Ebos, Y. Wu, L. Witte, S. Berry, M. Moore, and R. S. Kerbel. 2009. Dose-dependent increases in circulating TGF-alpha and other EGFR ligands act as pharmacodynamic markers for optimal biological dosing of cetuximab and are tumor independent. Clinical cancer research : an official journal of the American Association for Cancer Research 15:2397-2405.

Nauerth, M., B. Weissbrich, R. Knall, T. Franz, G. Dossinger, J. Bet, P. J. Paszkiewicz, L. Pfeifer, M. Bunse, W. Uckert, R. Holtappels, D. Gillert-Marien, M. Neuenhahn, A. Krackhardt, M. J. Reddehase, S. R. Riddell, and D. H. Busch. 2013. TCR-ligand koff rate correlates with the protective capacity of antigen-specific CD8⁺ T cells for adoptive transfer. Science translational medicine 5:192ra187.

O’Connor, C. M., S. Sheppard, C. A. Hartline, H. Huls, M. Johnson, S. L. Palla, S. Maiti, W. Ma, R. E. Davis, S. Craig, D. A. Lee, R. Champlin, H. Wilson, and L. J. Cooper. 2012. Adoptive T-cell therapy improves treatment of canine non-Hodgkin lymphoma post chemotherapy. Scientific reports 2:249.

Parsons, D. W., S. Jones, X. Zhang, J. C. Lin, R. J. Leary, P. Angenendt, P. Mankoo, H. Carter, I. M. Siu, G. L. Gallia, A. Olivi, R. McLendon, B. A. Rasheed, S. Keir, T. Nikolskaya, Y. Nikolsky, D. A. Busam, H. Tekleab, L. A. Diaz, Jr., J. Hartigan, D. R. Smith, R. L. Strausberg, S. K. Marie, S. M. Shinjo, H. Yan, G. J. Riggins, D. D. Bigner, R. Karchin, N. Papadopoulos, G. Parmigiani, B. Vogelstein, V. E. Velculescu, and K. W. Kinzler. 2008. An integrated genomic analysis of human glioblastoma multiforme. Science 321:1807-1812.

Paulos, C. M., M. M. Suhoski, G. Plesa, T. Jiang, S. Basu, T. N. Golovina, S. Jiang, N. A. Aqui, D. J. Powell, Jr., B. L. Levine, R. G. Carroll, J. L. Riley, and C. H. June. 2008. Adoptive immunotherapy: good habits instilled at youth have long-term benefits. Immunologic research 42:182-196.

Peng, W., Y. Ye, B. A. Rabinovich, C. Liu, Y. Lou, M. Zhang, M. Whittington, Y. Yang, W. W. Overwijk, G. Lizee, and P. Hwu. 2010. Transduction of tumor-specific T cells with CXCR2 chemokine receptor improves migration to tumor and antitumor immune responses. Clinical cancer research : an official journal of the American Association for Cancer Research 16:5458-5468.

Porter, D. L., B. L. Levine, M. Kalos, A. Bagg, and C. H. June. 2011. Chimeric antigen receptor-modified T cells in chronic lymphoid leukemia. The New England journal of medicine 365:725-733.

Rabinovich, P. M., M. E. Komarovskaya, Z. J. Ye, C. Imai, D. Campana, E. Bahceci, and S. M. Weissman. 2006. Synthetic messenger RNA as a tool for gene therapy. Human gene therapy 17:1027-1035.

Remington’s Pharmaceutical Sciences, 16th Ed., Mack, ed. (1980).

Robbins, P. F., M. E. Dudley, J. Wunderlich, M. El-Gamil, Y. F. Li, J. Zhou, J. Huang, D. J. Powell, Jr., and S. A. Rosenberg. 2004. Cutting edge: persistence of transferred lymphocyte clonotypes correlates with cancer regression in patients receiving cell transfer therapy. J Immunol 173:7125-7130.

Robert, P., M. Aleksic, O. Dushek, V. Cerundolo, P. Bongrand, and P. A. van der Merwe. 2012. Kinetics and mechanics of two-dimensional interactions between T cell receptors and different activating ligands. Biophysical journal 102:248-257.

Roberts et al., Immunol. Lett., 43:39-43, 1994.

Robins, H. S., P. V. Campregher, S. K. Srivastava, A. Wacher, C. J. Turtle, O. Kahsai, S. R. Riddell, E. H. Warren, and C. S. Carlson. 2009. Comprehensive assessment of T-cell receptor beta-chain diversity in alphabeta T cells. Blood 114:4099-4107.

Rosette, C., G. Werlen, M. A. Daniels, P. O. Holman, S. M. Alam, P. J. Travers, N. R. Gascoigne, E. Palmer, and S. C. Jameson. 2001. The impact of duration versus extent of TCR occupancy on T cell activation: a revision of the kinetic proofreading model. Immunity 15:59-70.

Rushworth, D. J., B.; Olivares, S.; Maiti, S.; Briggs, N.; Somanchi, S.; Dai, J.; Lee, D.A.; Cooper, L.J.N. 2014. Universal artificial antigen presenting cells to selectively propagate T cells expressing chimeric antigen receptors independent of specificity. Journal of Immunotherapy.

Schaft, N., J. Dorrie, I. Muller, V. Beck, S. Baumann, T. Schunder, E. Kampgen, and G. Schuler. 2006. A new way to generate cytolytic tumor-specific T cells: electroporation of RNA coding for a T cell receptor into T lymphocytes. Cancer immunology, immunotherapy : CII 55:1132-1141.

Schamel, W. W., and B. Alarcon. 2013. Organization of the resting TCR in nanoscale oligomers. Immunological reviews 251:13-20.

Schamel, W. W., I. Arechaga, R. M. Risueno, H. M. van Santen, P. Cabezas, C. Risco, J. M. Valpuesta, and B. Alarcon. 2005. Coexistence of multivalent and monovalent TCRs explains high sensitivity and wide range of response. The Journal of experimental medicine 202:493-503.

Schneider, J. Embryol. Exp. Morph. 1972 Vol 27:353-365.

Singh, H., M. J. Figliola, M. J. Dawson, S. Olivares, L. Zhang, G. Yang, S. Maiti, P. Manuri, V. Senyukov, B. Jena, P. Kebriaei, R. E. Champlin, H. Huls, and L. J. Cooper. 2013. Manufacture of clinical-grade CD19-specific T cells stably expressing chimeric antigen receptor using Sleeping Beauty system and artificial antigen presenting cells. PloS one 8:e64138.

Singh, H., P. R. Manuri, S. Olivares, N. Dara, M. J. Dawson, H. Huls, P. B. Hackett, D. B. Kohn, E. J. Shpall, R. E. Champlin, and L. J. Cooper. 2008. Redirecting specificity of T-cell populations for CD19 using the Sleeping Beauty system. Cancer research 68:2961-2971.

Smith, J. S., I. Tachibana, S. M. Passe, B. K. Huntley, T. J. Borell, N. Iturria, J. R. O’Fallon, P. L. Schaefer, B. W. Scheithauer, C. D. James, J. C. Buckner, and R. B. Jenkins. 2001. PTEN mutation, EGFR amplification, and outcome in patients with anaplastic astrocytoma and glioblastoma multiforme. Journal of the National Cancer Institute 93:1246-1256.

Stancovski, I. et al., J.Immunol. 151:6577, 1993.

Stephan, M. T., V. Ponomarev, R. J. Brentjens, A. H. Chang, K. V. Dobrenkov, G. Heller, and M. Sadelain. 2007. T cell-encoded CD80 and 4-1BBL induce auto- and transcostimulation, resulting in potent tumor rejection. Nature medicine 13:1440-1449.

Stone, J. D., A. S. Chervin, and D. M. Kranz. 2009. T-cell receptor binding affinities and kinetics: impact on T-cell activity and specificity. Immunology 126:165-176.

Stone, J. D., and D. M. Kranz. 2013. Role of T cell receptor affinity in the efficacy and specificity of adoptive T cell therapies. Frontiers in immunology 4:244.

Suhoski, M. M., T. N. Golovina, N. A. Aqui, V. C. Tai, A. Varela-Rohena, M. C. Milone, R. G. Carroll, J. L. Riley, and C. H. June. 2007. Engineering artificial antigen-presenting cells to express a diverse array of co-stimulatory molecules. Molecular therapy : the journal of the American Society of Gene Therapy 15:981-988.

Sun, J. C., and M. J. Bevan. 2003. Defective CD8 T cell memory following acute infection without CD4 T cell help. Science 300:339-342.

Szerlip, N. J., A. Pedraza, D. Chakravarty, M. Azim, J. McGuire, Y. Fang, T. Ozawa, E. C. Holland, J. T. Huse, S. Jhanwar, M. A. Leversha, T. Mikkelsen, and C. W. Brennan. 2012. Intratumoral heterogeneity of receptor tyrosine kinases EGFR and PDGFRA amplification in glioblastoma defines subpopulations with distinct growth factor response. Proceedings of the National Academy of Sciences of the United States of America 109:3041-3046.

Talavera, A., R. Friemann, S. Gomez-Puerta, C. Martinez-Fleites, G. Garrido, A. Rabasa, A. Lopez-Requena, A. Pupo, R. F. Johansen, O. Sanchez, U. Krengel, and E. Moreno. 2009. Nimotuzumab, an antitumor antibody that targets the epidermal growth factor receptor, blocks ligand binding while permitting the active receptor conformation. Cancer research 69:5851-5859.

Tian, S., R. Maile, E. J. Collins, and J. A. Frelinger. 2007. CD8⁺ T cell activation is governed by TCR-peptide/MHC affinity, not dissociation rate. J Immunol 179:2952-2960.

Till, B. G., M. C. Jensen, J. Wang, E. Y. Chen, B. L. Wood, H. A. Greisman, X. Qian, S. E. James, A. Raubitschek, S. J. Forman, A. K. Gopal, J. M. Pagel, C. G. Lindgren, P. D. Greenberg, S. R. Riddell, and O. W. Press. 2008. Adoptive immunotherapy for indolent non-Hodgkin lymphoma and mantle cell lymphoma using genetically modified autologous CD20-specific T cells. Blood 112:2261-2271.

Topalian and Rosenberg, Acta Haematol., 78(Suppl 1):75-76, 1987.

Torikai, H., A. Reik, P. Q. Liu, Y. Zhou, L. Zhang, S. Maiti, H. Huls, J. C. Miller, P. Kebriaei, B. Rabinovitch, D. A. Lee, R. E. Champlin, C. Bonini, L. Naldini, E. J. Rebar, P. D. Gregory, M. C. Holmes, and L. J. Cooper. 2012. A foundation for universal T-cell based immunotherapy: T cells engineered to express a CD19-specific chimeric-antigen-receptor and eliminate expression of endogenous TCR. Blood 119:5697-5705.

Turatti, F., M. Figini, E. Balladore, P. Alberti, P. Casalini, J. D. Marks, S. Canevari, and D. Mezzanzanica. 2007. Redirected activity of human antitumor chimeric immune receptors is governed by antigen and receptor expression levels and affinity of interaction. J Immunother 30:684-693.

Turkman, N., A. Shavrin, R. A. Ivanov, B. Rabinovich, A. Volgin, J. G. Gelovani, and M. M. Alauddin. 2011. Fluorinated cannabinoid CB2 receptor ligands: synthesis and in vitro binding characteristics of 2-oxoquinoline derivatives. Bioorganic & medicinal chemistry 19:5698-5707.

Vartanian, A., S. K. Singh, S. Agnihotri, S. Jalali, K. Burrell, K. D. Aldape, and G. Zadeh. 2014. GBM’s multifaceted landscape: highlighting regional and microenvironmental heterogeneity. Neuro-oncology.

Viola, A., and A. Lanzavecchia. 1996. T cell activation determined by T cell receptor number and tunable thresholds. Science 273:104-106.

Weijtens, M. E. et al., J.Immunol. 157:836, 1996.

Weijtens, M. E., E. H. Hart, and R. L. Bolhuis. 2000. Functional balance between T cell chimeric receptor density and tumor associated antigen density: CTL mediated cytolysis and lymphokine production. Gene therapy 7:35-42.

Wilkie, S., M. C. van Schalkwyk, S. Hobbs, D. M. Davies, S. J. van der Stegen, A. C. Pereira, S. E. Burbridge, C. Box, S. A. Eccles, and J. Maher. 2012. Dual targeting of ErbB2 and MUC1 in breast cancer using chimeric antigen receptors engineered to provide complementary signaling. Journal of clinical immunology 32:1059-1070.

Wu, F., W. Zhang, H. Shao, H. Bo, H. Shen, J. Li, Y. Liu, T. Wang, W. Ma, and S. Huang. 2013. Human effector T cells derived from central memory cells rather than CD8(+)T cells modified by tumor-specific TCR gene transfer possess superior traits for adoptive immunotherapy. Cancer letters 339:195-207.

Yano, S., K. Kondo, M. Yamaguchi, G. Richmond, M. Hutchison, A. Wakeling, S. Averbuch, and P. Wadsworth. 2003. Distribution and function of EGFR in human tissue and the effect of EGFR tyrosine kinase inhibition. Anticancer research 23:3639-3650.

Yokosuka, T., and T. Saito. 2010. The immunological synapse, TCR microclusters, and T cell activation. Current topics in microbiology and immunology 340:81-107.

Yoon, S. H., J. M. Lee, H. I. Cho, E. K. Kim, H. S. Kim, M. Y. Park, and T. G. Kim. 2009. Adoptive immunotherapy using human peripheral blood lymphocytes transferred with RNA encoding Her-2/neu-specific chimeric immune receptor in ovarian cancer xenograft model. Cancer gene therapy 16:489-497.

Zehn, D., S. Y. Lee, and M. J. Bevan. 2009. Complete but curtailed T-cell response to very low-affinity antigen. Nature 458:211-214.

Zhang, M., S. Maiti, C. Bernatchez, H. Huls, B. Rabinovich, R. E. Champlin, L. M. Vence, P. Hwu, L. Radvanyi, and L. J. Cooper. 2012. A new approach to simultaneously quantify both TCR alpha- and beta-chain diversity after adoptive immunotherapy. Clinical cancer research : an official journal of the American Association for Cancer Research 18:4733-4742.

Zhao, Y., E. Moon, C. Carpenito, C. M. Paulos, X. Liu, A. L. Brennan, A. Chew, R. G. Carroll, J. Scholler, B. L. Levine, S. M. Albelda, and C. H. June. 2010. Multiple injections of electroporated autologous T cells expressing a chimeric antigen receptor mediate regression of human disseminated tumor. Cancer research 70:9053-9061.

Zhong, S., K. Malecek, L. A. Johnson, Z. Yu, E. Vega-Saenz de Miera, F. Darvishian, K. McGary, K. Huang, J. Boyer, E. Corse, Y. Shao, S. A. Rosenberg, N. P. Restifo, I. Osman, and M. Krogsgaard. 2013. T-cell receptor affinity and avidity defines antitumor response and autoimmunity in T-cell immunotherapy. Proceedings of the National Academy of Sciences of the United States of America 110:6973-6978.

Zhou, X., J. Li, Z. Wang, Z. Chen, J. Qiu, Y. Zhang, W. Wang, Y. Ma, N. Huang, K. Cui, and Y. Q. Wei. 2013. Cellular immunotherapy for carcinoma using genetically modified EGFR-specific T lymphocytes. Neoplasia 15:544-553.

Zuckier, L. S., E. Z. Berkowitz, R. J. Sattenberg, Q. H. Zhao, H. F. Deng, and M. D. Scharff. 2000. Influence of affinity and antigen density on antibody localization in a modifiable tumor targeting model. Cancer research 60:7008-7013.

What is claimed is:

1-92. (canceled)

93. A transgenic human T cell comprising an expressed chimeric antigen receptor (CAR) targeted to EGFR, said CAR comprising CDR sequences of nimotuzumab, wherein VL CDR1 comprises RSSQNIVHSNGNTYLD (SEQ ID NO: 5); VL CDR2 comprises KVSNRFS (SEQ ID NO: 6); VL CDR3 comprises FQYSHVPWT (SEQ ID NO: 7); VH CDR1 comprises NYYIY (SEQ ID NO: 8); VH CDR2 comprises GINPTSGGSNFNEKFKT (SEQ ID NO: 9) and VH CDR3 comprises QGLWFDSDGRGFDF (SEQ ID NO: 10), said T cell exhibiting selective cytotoxicity to an EGFR overexpressing cancer cell.

94. The T cell of claim 93, wherein the CAR comprises a sequence having at least about 90% identity with the amino acid sequence of SEQ ID NO: 1 and/or SEQ ID NO: 2.

95. The T cell of claim 93, wherein the CAR comprises the antigen binding portions of SEQ ID NO: 1 and SEQ ID NO: 2.

96. The T cell of claim 93, further comprising a membrane bound IL-15.

97. The T cell of claim 93, further comprising a Sleeping Beauty transposase.

98. The T cell of claim 93, wherein the CAR is expressed in a Sleeping Beauty transposon.

99. A pharmaceutical composition comprising the T cell of claim 98 in a pharmaceutically acceptable carrier.

100. The pharmaceutical composition of claim 99, comprising between about 1×10³ and 1×10⁸ T cells in a pharmaceutically acceptable carrier.

101. A method of treating a patient having a cancer that overexpresses EGFR, the method comprising administering to the patient an effective amount of the pharmaceutical composition of claim 99.

102. The method of claim 101, wherein the cancer is a glioma.

103. An isolated transgenic cell comprising an expressed chimeric T-cellreceptor (CAR) targeted to an antigen, said CAR having a K_{d} of between about 5 nM and about 500 nM relative to the antigen.

104. The isolated transgenic cell of claim 103, wherein the CAR comprises a sequence having at least about 90% identity with the amino acid sequence of SEQ ID NO: 1 and/or SEQ ID NO: 2.

105. The isolated transgenic cell of claim 103, wherein the CAR comprises the antigen binding portions of SEQ ID NO: 1 and SEQ ID NO: 2.

106. The isolated transgenic cell of claim 103, further comprising a membrane bound IL-15.

107. The isolated transgenic cell of claim 103, further comprising a Sleeping Beauty transposase.

108. The isolated transgenic cell of claim 103, wherein the CAR is expressed in a Sleeping Beauty transposon.

109. A pharmaceutical composition comprising the isolated transgenic cell of claim 108 in a pharmaceutically acceptable carrier.

110. The pharmaceutical composition of claim 109, comprising between about 1x10³ and 1×10⁸ transgenic cells in a pharmaceutically acceptable carrier.

111. A method of treating a patient having a cancer that overexpresses EGFR, the method comprising administering to the patient an effective amount of the pharmaceutical composition of claim 109.

112. A method of treating a cancer in a subject in need thereof, the method comprising administering a composition comprising an effective amount of chimeric antigen receptor (CAR) T cells to provide a T-cell response that selectively targets cancer cells having elevated expression of an antigen, wherein the CAR T cells comprise an expressed CAR that binds to the antigen, said CAR T cells having:
(i) cytotoxic activity only upon multivalent binding of the antigen by the T cells; or
(ii) a CAR having a K_{d} of between about 5 nM and about 500 nM relative to the antigen.
